(19) United States
(12) Patent Application Publication
(10) Pub. No.: US 2006/0234242 A1
(43) Pub. Date: Oct. 19, 2006

(54) Methods for identifying therapeutic targets involved in glucose and lipid metabolism

(75) Inventors: Richard Bentley Cheatham, Durham, NC (US); Barry Steven Henderson, Hillsborough, NC (US); William C. Phelps, Durham, NC (US)

Correspondence Address: SULLIVAN & WORCESTER LLP, ONE POST OFFICE SQUARE, BOSTON, MA, 02109, US

(73) Assignee: Ribonomics, Inc., Durham, NC (US)

(21) Appl. No.: 10/552,642

(22) PCT Filed: Apr. 7, 2004
(86) PCT No.: PCT/US04/10686

§ 371 Date: May 19, 2006

Related U.S. Application Data
(60) Provisional application No. 60/461,016, filed on Apr. 7, 2003.

(51) Int. Cl. C12Q 1/68

(52) U.S. Cl. 435/6

(57) ABSTRACT

The identification and evaluation of mRNA and protein targets associated with RNA binding proteins or mRNP complexes is described. In particular, the invention provides methods for identifying RNA binding proteins associated with physiological pathways that participate in glucose and lipid metabolism and mRNAs that exhibit coordinated gene regulation across those M pathways. Candidate targets are provided that are useful for the diagnosis or treatment of diseases related to diseases, such as disease related to aberrant glucose and lipid metabolism, such as, for example, obesity, diabetes, and hypoglycemia.

### RELATED APPLICATIONS

This application claims priority to and the benefit of U.S. Ser. No. 60/461,016, filed Apr. 7, 2003, the contents of which are incorporated by reference herein.

### FIELD OF THE INVENTION

The invention provides methods and compositions for identifying and characterizing functionally related gene products associated with isolated mRNP complexes. The invention also provides methods and compositions for identifying and characterizing metabolic pathways, such as glucose or lipid metabolic pathways, and therapeutic targets and therapeutics for treating diseases associated with metabolic pathways.

### BACKGROUND OF THE INVENTION

Glucose and lipid metabolism are regulated by the coordinated expression of a number of proteins that participate in insulin production, secretion, and action. Beta cells of the pancreas sense increased plasma glucose, lipids, and other nutrients, and activate a cascade of intracellular reactions leading to the controlled release of insulin from storage granules. Insulin, in turn, controls plasma glucose and lipid levels by stimulating glucose uptake into insulin-sensitive tissues (e.g.e.g., skeletal muscle and adipose), lipid metabolism, and inhibiting hepatic glucose production.

Diabetes is a disease characterized by an impairment of insulin action. Type 1 diabetes results from an inability of pancreatic beta cells to produce insulin, forcing patients to take daily insulin injections to control their blood glucose. Type 2 diabetes is a metabolic disorder in which a patient becomes resistant to insulin's actions, leading to hyperglycemia, hyperlipidemia, and hyperinsulinemia. In many cases, Type 2 diabetes is associated with obesity and a sedentary lifestyle. Efforts have been made to establish pancreatic beta cell lines from adult and embryonic stem cells and to engineer pancreatic beta cell-like cell lines in order to study the metabolic pathways that are activated during development, growth, and maintenance of pancreatic beta cells.

Although some of the cellular pathways involved in glucose and lipid metabolism are understood, a number of regulatory aspects of those pathways have not been fully characterized. The identification of RNAs that are co-regulated with insulin gene expression would provide information about the regulation of genes involved in controlling insulin production and secretion by beta cells of the pancreas. Identification of co-expressed RNAs would also help identify previously unknown components of the insulin signaling pathway and other glucose and/or lipid metabolic pathways in adipocytes, as well as other cells that participate in glucose or lipid metabolism. Identification of the components of glucose and lipid metabolic pathways provides new therapeutic targets for diabetes, obesity, and other diseases characterized by altered glucose or lipid metabolism. A need therefor exists for a sensitive, focused, and efficient method for identifying such functionally related genes, therapeutic targets, and therapeutics.

### SUMMARY OF THE INVENTION

The invention exploits the ability of RNA binding proteins to bind and coordinate the expression of functionally and structurally related RNAs. The RNAs bound to a particular RNA binding protein define a cluster of functionally related gene products and may also possess common primary and/or secondary structures that mediate binding to the RNA binding protein. RNA binding proteins and RNAs identified by methods of the invention are useful for elucidating physiological or regulatory pathways, such as glucose or lipid metabolic pathways, including insulin action, insulin resistance, obesity, and diabetes. The RNAs, the genes encoding those RNAs, and proteins identified by the methods of the invention are putative therapeutic targets due to their ability to regulate other genes that participate in, or otherwise modulate, aberrant physiological, metabolic or regulatory pathways in a disease state.

The invention provides a ribonomic profile, and methods for identifying and characterizing a ribonomic profile, including the expression of RNAs, RNA binding proteins, and mRNP complex-associated proteins associated with a particular mRNP complex or set of mRNP complexes. For example, genes participating in a glucose or a lipid metabolic pathway are identified by characterizing the mRNAs associated with a particular mRNP complex known, or determined, to be a participant in the pathway. According to the invention, mRNAs or proteins are classified into biologically relevant subsets on the basis of structural and/or functional relationships (e.g. e.g., that participate in the same insulin production or secretion pathway, or that facilitate gene expression during growth and development in normal or diseased pancreatic beta cells). In contrast to the static genomics and proteomics approaches to gene characterization and drug discovery, this “ribonomics” approach provides a dynamic snapshot of the flow of genetic information at a given time in the life of a cell or tissue, for example, in a normal or diseased state or in response to an environmental influence, such as glucose or a drug.

In an aspect, the invention provides methods for identifying RNA binding protein, mRNA and protein components of an mRNP complex in cells associated with a physiological process or pathway, by immunoprecipitating an mRNP complex, identifying and comparing the components of the mRNP complex, such as, for example, RNA binding proteins, mRNAs, and other proteins, and validating the biological role of those proteins, or the genes that encode those proteins, in the physiological process or pathway. In an embodiment, the method further includes preparing an RNA binding protein profile, isolating the RNA binding protein, and/or producing antibodies to the RNA binding protein.

In one aspect, the invention provides methods of identifying a therapeutic target related to the treatment of a disease, such as aberrant glucose or lipid metabolism. The protein or RNA levels of at least one component of an isolated mRNA ribonucleoprotein (mRNP) complex in a cell sample is measured and compared to the levels of the protein or RNA levels of the component in a second cell sample. The two cell samples may differ in that one is normal and one is diseased or may differ regarding their state of differentiation. The cell samples may also differ in that one sample is treated with an agent and one sample is not. For example, the cell samples may contain mostly mature adipocytes, preadipocytes, pancreatic beta cells, hepatocytes, skeletal muscle cells, or cardiac muscle cells, or any cell that participates in glucose or insulin metabolism, for example. If the levels of the component in the first sample are different from the levels of the component in the second sample, the component, a nucleic acid that encodes the component (if the component is a protein), or a protein encoded by the component (if the component is a nucleic acid) is a potential therapeutic target for the treatment of a disease related to altered glucose or lipid metabolism. In an embodiment, the component is an RNA binding protein, an RNA, or an mRNP-associated protein.

In an embodiment, the first cell sample has the phenotype of a mature adipocyte and the second cell sample has the phenotype of a preadipocyte. A difference in the expression of a component of the mRNP complex between the two cell types is indicative that the component participates in a pathway involved in the differentiation from preadipocyte to adipocyte.

In another embodiment, the first cell sample has a disease phenotype related to glucose or lipid metabolism, such as obesity, diabetes, hypoglycemia, glucotoxicity, lipidtoxicity, insulin-resistance, hyperlipidemia, and lipodystrophy, and the second cell sample has a normal phenotype.

In another embodiment, the method has an additional step of treating the sample with an agent prior to measuring the protein or RNA levels of the mRNP complex component, wherein the agent alters the levels of at least one component of a glucose metabolic or a lipid metabolic pathway. In an embodiment, the agent is insulin, glucose, insulin-like growth factor-1 (IGF-1), a β-adrenergic agonist, glucagon-like peptide-1 (GLP-1), fatty acid, a peroxisome proliferator activated receptor (PPAR) ligand, or insulin-like growth factor 2 (IGF-2), RNAi against an RNA binding protein, overexpression of an RNA binding protein, or an enhancer of an RNA binding protein for example. In another embodiment, the agent is a test therapeutic, such as, for example, a nucleic acid, a hormone, an antibody, an antibody fragment, an antigen, a cytokine, a growth factor, a pharmacological agent (e.g. e.g., chemotherapeutic, carcinogenic, or other cell), a chemical composition, a protein, a peptide, and/or a small molecule (e.g., a putative drug).

In an aspect, the invention comprises methods for identifying RNA binding protein, mRNA and protein components of an mRNP complex in cells associated with physiological pathways or processes, for example glucose or lipid metabolism. The method includes the steps of identifying RNA binding proteins enriched in cells, such as, for example, adipocytes or preadipocytes (for example in lean or obese individuals), treating the cells with an agent, such as, for example, insulin or a beta 3 agonist, and identifying the components of the mRNP complex (e.g., functional cluster). In an embodiment, the methods of the invention further include the step of identifying a suitable RNA binding protein for analysis, e.g., an RNA binding protein that participates in the regulation of the physiological pathway or process. In a further embodiment, the method further includes the step of validating the function of the component within the pathway.

In another embodiment, the methods of the invention have a further step of isolating the component, a nucleic acid encoding the component, or a protein encoded by the component. For example, the methods of the invention can identify and isolate an mRNA encoding the RNA binding protein and/or an mRNP complex-associated protein, a gene encoding the RNA binding protein and/or an mRNP complex-associated protein, an mRNP complex comprising the RNA binding protein and/or an mRNP complex-associated protein, an mRNA associated with the mRNP complex, and a gene encoding the mRNA associated with the mRNP complex. In addition, the invention contemplates identifying other associated RNAs that bind to one or more components of the mRNP complex. These RNAs include, but are not limited to, microRNA (miRNA), non-coding RNA (ncRNA or snmRNA), ribosomal RNA (rRNA), small interfering RNA (siRNA), small nuclear RNA (snRNA), small nuclear RNA (snoRNA), small temporal RNA (stRNA), and transfer RNA (tRNA).

In an embodiment, the component is an RNA binding protein, such as Polypyrimidine Tract Binding Protein (PTB, also known as RNA binding protein 1 (RBP1)). In another embodiment, the RNA binding protein is selected from the group consisting of the RNA binding proteins identified in FIGS. 10-22. These RNAs were subjected to analysis on a microarray containing RNA binding protein genes. These genes and their encoded proteins represent candidate therapeutic targets as well as candidates for RAS™ analysis for elucidation of cellular pathways involved in glucose and lipid metabolism, insulin action, insulin resistance, diabetes and obesity, for example. In an embodiment, the RNA binding protein has a tag (e.g.e.g., HIS [symbol] GST) to facilitate affinity purification.

In an embodiment, the component is an mRNA that is associated with a particular RNA binding protein. The mRNA are identified singly or mRNAs are identified en masse, e.g., using arrays containing a number of probes. In an embodiment, the mRNA encodes a kinase, a transporter, a phosphatase, a channel protein, a protease, a receptor, a transcription factor, or a transferase. For example, the protein may be 3-phosphoinositide dependent protein kinase-1; nuclear ubiquitous casein kinase 2; neural receptor protein-tyrosine kinase; MAP-kinase activating death domain; AMP-activated protein kinase beta-2 regulatory subunit; calcium/calmodulin-dependent protein kinase IV; Protein kinase C beta; adenylate kinase 3; mitogen activated protein kinase; kinase 5; 6-phosphofructo-2-kinase/fructose-2,6-bisphosphatase 2; phosphatidylinositol 4-kinase; Glucokinase; glycogen synthase kinase 3 beta; phosphorylase kinase (gamma 2, testis); protein tyrosine phosphatase (non-receptor type 1); protein tyrosine phosphatase (non-receptor type 5); inositol polyphosphate-5-phosphatase D; Protein tyrosine phosphatase (receptor-type, zeta polypeptide); dual specificity phosphatase 6; protein tyrosine phosphatase (non-receptor type 12); glucose-6-phosphatase (catalytic); 6-phosphofructo-2-kinase/fructose-2,6-bisphosphatase 2; proton gated cation channel DRASIC; Sodium channel (nonvoltage-gated 1, alpha (epithelial)); calcium channel (voltage-dependent, alpha2/delta subunit 1); Potassium inwardly-rectifying (channel, subfamily J, member 6); potassium channel regulator 1; calcium channel (voltage-dependent, T type, alpha 1 G subunit) cyclic nucleotide-gated cation channel; amiloride-sensitive cation channel 1; potassium inwardly-rectifying channel J14; potassium large conductance calcium-activated channel (subfamily M, alpha member 1); potassium voltage gated channel (Shab-related subfamily, member 2); potassium channel subunit (Slack); potassium intermediate/small conductance calcium-activated channel (subfamily N, member 1); Sodium channel (voltage-gated, type V, alpha polypeptide); amiloride-sensitive cation channel 2 (neuronal); potassium channel (subfamily K, member 6 (TWIK-2)); cation-chloride cotransporter 6; solute carrier family 21 (organic anion transporter, member 12); amino acid transporter system A2; peptide/histidine transporter; choline transporter; solute carrier family 31 (copper transporters, member 1); solution carrier family 13 (sodium-dependent dicarboxylate transporter); solute carrier family 2 (facilitated glucose transporter, member 13); solute carrier family 12 (potassium-chloride transporter, member 5); Solute carrier family 6 (neurotransmitter transporter, serotonin, member 4); Solute carrier family 2 A2 (glucose transporter, type 2); carboxypeptidase D; ubiquitin specific protease 2; mast cell protease 1; proprotein convertase subtilisin/kexin, type 7; lamin [symbol] receptor 1 (67 kD, ribosomal protein SA); protein tyrosine phosphatase (non-receptor type 1); calcium-sensing receptor; neural receptor protein-tyrosine kinase; glutamate receptor (metabotropic 4); nuclear receptor subfamily 4 (group A, member 2); Neuropeptide Y5 receptor protein tyrosine phosphatase (non-receptor type 5); insulin-like growth factor 1 receptor; Protein tyrosine phosphatase (receptor-type, zeta polypeptide); nuclear receptor subfamily 4 (group A, member 3); glutamate receptor (metabotropic 1); Tumor necrosis factor receptor superfamily (member 1a); insulin receptor; gamma-aminobutyric acid receptor associated protein; protein tyrosine phosphatase; non-receptor type 12; cholinergic receptor (nicotinic, beta polypeptide 1 olfactory receptor (U131); Gamma-aminobutyric acid receptor beta 2; glial cell line derived neurotrophic factor family receptor alpha 1; Glycine receptor beta; glutamate receptor interact [symbol] protein 2; adenylate cyclase activating polypeptide 1 receptor 1; asialoglycoprotein receptor 2; adenosine A3 receptor; Fibroblast growth factor receptor 1; nuclear receptor binding factor 2; purinergic receptor P2Y (G-protein coupled 1); nuclear receptor subfamily 1 (group H, member 4); peroxisome proliferator activator receptor (gamma); 5 hydroxytryptamine (serotonin) receptor 4; retinoid X receptor gamma; insulin receptor-related receptor; putative N-acetyltransferase Camello 4; lecithin-retinol acyltransferase; Phenylethanolamine N-methyltransferase; fucosyltransferase 2; Sialyltransferase 8 (GT3 alpha 2,8-sialyltransferase) C; UDP-glucuronosyltransferase; alpha 1,3-fucosyltransferase Fuc-T (similar to mouse Fut4); diacylglycerol O-acyltransferase 1; signal transducer and activator of transcription 3; ISL1 transcription factor (LIM/homeodomain); and oligodendrocyte transcription factor 1. In another [symbol] embodiment, the protein is encoded by a gene selected from the group consisting of CNCG, CACNA2D1, KCNC3, and KCNB2.

In another aspect, the invention provides a method for identifying a therapeutic target for [symbol] the treatment of a disease that involves a physiological or regulatory pathway, such as aberrant [symbol] glucose metabolism or lipid metabolism, by comparing RNA or protein levels of at least one component of an isolated mRNP complex in a sample from an individual with a disease associated with altered glucose metabolism or lipid metabolism to RNA or protein levels of the [symbol] component in a healthy sample. If the levels of the component in the diseased sample are different from the levels of the component in the healthy sample, the component, a nucleic acid [symbol] that encodes the component, or a protein encoded by the component is a potential therapeutic target for the treatment of the disease.

In another aspect, the invention provides a method for identifying a gene or gene produce [symbol] involved in a physiological or regulatory pathway in a cell, such as a glucose or lipid metabolic [symbol] pathway. For example, an mRNP complex containing at least one component that participates a glucose metabolic or lipid metabolic pathway is isolated and at least one additional component [symbol] of the isolated mRNP complex is identified. The additional component is also likely involved a glucose or lipid metabolic pathway. In an embodiment, the method includes the step of confirming the activity of the additional component by inhibiting the expression of the additional [symbol] component in a cell or organism and determining the effect of the inhibition on glucose metabolism or lipid metabolism. Inhibition can be achieved by any number of means, including [symbol] for example, inhibiting gene expression of the additional component using an RNAi, an antisense [symbol] RNA, a ribozyme, a PNA, or an antibody.

In another aspect, the invention provides a method for identifying an agent that alters a physiological or regulatory pathway in a cell, such as a glucose metabolism or lipid metabolism [symbol] A cell sample is treated with an agent and an mRNP complex having at least one component the [symbol] participates in a metabolic pathway, for example, a glucose metabolic or lipid metabolic pathway, is isolated from the sample, and the RNA or protein levels of at least one component the isolated mRNP complex are measured and compared to the RNA or protein levels of the component isolated from an untreated control sample. Differential expression of the component [symbol] in the agent-treated sample compared to the untreated control sample is indicative that the agent [symbol] regulates or participates in glucose metabolism or lipid metabolism. In an embodiment, the agent interacts with or regulates a component of a pathway, such as an insulin production pathway, a lipogenesis pathway, an insulin action pathway, a lipid metabolism pathway, or a glucose metabolism pathway, or any pathway that participates in an aspect of glucose and lipid [symbol] metabolism. In yet another embodiment, the agent inhibits a pathway. In another embodiment [symbol] the agent enhances a pathway. In an embodiment, the agent is insulin, a beta-adrenergic agonist [symbol] insulin-like growth factor-1 (IGF-1), glucagon-like peptide-1 (GLP-1), fatty acid, peroxisome proliferator activated receptor (PPAR) ligands (e.g., thiazolidinediones, fibrates, halogenated fatty acids, and tyrosine derivatives), insulin-like growth factor-2 (IGF-2), an RNAi against an RNA binding protein, an enhancer of RNA binding protein expression, and/or glucose.

In a particular aspect, the invention provides a method for identifying a gene product the [symbol] regulates glucose metabolism in a cell. The expression in an isolated mRNP complex of at least [symbol] one gene product of a pancreatic beta cell sample is measured. The gene product may be an RNA binding protein, an mRNA associated with the RNA binding protein, or an mRNP complex-associated protein. The cell sample, such as a pancreatic beta-cell sample, is then treated with an agent, such as, for example, insulin, glucose, insulin-like growth factor-1 (IGF-1), a β-adrenergic agonist, glucose, glucagon-like peptide-1 (GLP-1), fatty acid, a peroxisome proliferator activated receptor (PPAR) ligand, or insulin-like growth factor 2 (IGF-2). The expression of the gene product is then measured after treatment. A difference in the expression [symbol] of the gene product after treatment compared to the expression of the gene product before treatment is indicative that the gene product participates in the regulation of glucose metabolism.

[symbol]

In another aspect, the invention provides a method for identifying an agent that regulated [symbol] insulin production and/or its regulated secretion in a pancreatic beta cell. A pancreatic beta cell [symbol] sample is treated with a nucleic acid capable of binding to at least one RNA binding protein that [symbol] is capable of binding to a 3′ untranslated region or a 5′ untranslated region of a preproinsulin mRNA. The nucleic acid is then separated from the RNA binding protein and the RNA binding [symbol] protein is identified. In an embodiment, the RNA binding protein binds to a nucleic acid having [symbol] a sequence 5′-gaauaaaaccuuugaaagagcacuac-3′,5′-cccaccacuacccuguccaccccucugcaaug-3′, or 5 [symbol] agccctaagtgaccagctacagtcggaaaccatcagcaagcaggtcattgttccaac-3′.

In another embodiment, the invention provides a method for identifying a component of [symbol] an mRNP complex by transfecting a cell sample with a nucleic acid that inhibits the expression of an RNA binding protein associated with the mRNP complex. Total RNA from the cell same [symbol] and from a control sample is then isolated and measured. RNAs that have altered expression i [symbol] the nucleic acid-transfected sample compared to the control sample are considered members of [symbol] the mRNP complex that share functional and/or structural characteristics (e.g.e.g., that participate in the same metabolic pathway).

In another aspect, the invention provides an isolated mRNP complex, for example, an mRNP complex, containing polypyrimidine tract binding (PTB) and at least one mRNA associated with the PTB protein.

In another aspect, the invention provides methods for identifying a protein that regulate [symbol] insulin production and/or its regulated secretion by measuring the expression of an RNA binding [symbol] protein, an mRNA associated with the RNA binding protein, and/or an mRNP complex-associated protein in a pancreatic beta cell sample, treating the pancreatic beta cell sample with [symbol] an agent, such as, insulin, a beta-adrenergic agonist, insulin-like growth factor-1 (IGF-1), glucagon-like peptide 1 (GLP-1), fatty acid, peroxisome proliferator activated receptor (PPAR [symbol] ligands (e.g., thiazolidinediones, fibrates, halogenated fatty acids, and tyrosine derivatives), insulin-like growth factor-2 (IGF-2), RNAi against an RNA binding protein involved in insulin [symbol] production or secretion, an enhancer of an RNA binding protein expression and/or glucose, and [symbol] measuring expression of the levels of RNA binding protein, mRNA, and/or an mRNP complex [symbol] associated protein after treatment. The difference in the expression of the RNA binding protein [symbol] an mRNA associated with the RNA binding protein, and/or an mRNP complex-associated protein after treatment compared to expression before treatment is indicative that the RNA binding protein, mRNA, associated with the RNA binding protein, and/or an mRNP complex-associated protein regulates insulin production.

In another aspect, the invention provides methods of identifying gene products co-regulated with an mRNA that participates in the glucose or lipid metabolic pathway, such as, f [symbol] example, preproinsulin mRNA, by isolating an RNA binding protein or mRNP complex-associated protein that binds to the mRNA known to participate in glucose or lipid metabolism and identifying at least one additional component of the mRNP complex (e.g., mRNA, RNA binding protein, and/or mRNP complex-associated protein).

In another aspect, the invention provides methods for assessing the efficacy of an agent [symbol] as a therapeutic for treating an individual having a disease associated with altered glucose and/or [symbol] lipid metabolism. The methods comprise the steps of contacting a sample from an individual having a disease with an agent, and comparing the level of expression of an RNA binding protein, an mRNA associated with the RNA binding protein, or an mRNP complex-associated protein in the agent-treated sample to the level of expression of the RNA binding protein, the mRNA associated with the RNA binding protein, or the mRNP complex-associated protein in [symbol] control sample, wherein a difference in expression is indicative that the agent is a candidate therapeutic capable of treating the disease. The methods of the invention are also used to monitor the efficacy or toxicity of an agent.

In another aspect, the invention provides a method to identify genes affected by the activity of a specific RNA binding protein. RNAi-mediated gene silencing is used to inhibit the [symbol] expression of a specific RNA binding protein. RNA samples are isolated from control RNAi treated cells or tissues and RNA binding protein-specific RNAi treated cells or tissues and gene [symbol] that are differentially expressed are identified.

The foregoing and other objects, features and advantages of the present invention will be [symbol] made more apparent from the following drawings and detailed description of preferred embodiments of the invention.

### BRIEF DESCRIPTION OF THE DRAWINGS

The objects and features of the invention may be better understood by reference to the drawings described below in which,

FIG. 1 is a schematic overview outlining an embodiment of the RIBOTRAP™ assay for the isolation of an RNA binding protein (RBP-X) binding to a biotinylated mRNA of interest [symbol] using a streptavidin-agarose support.

FIG. 2 is a schematic overview of the RNA binding protein identification using one type of RIBOTRAP™ assay and subsequent RAS™ assay for identification of mRNA substrate [symbol] for the RNA binding protein identified by RIBOTRAP™.

FIG. 3 shows the general scheme of Ribonomic Analysis System, RAS™. RAS™ involves the isolation of mRNP complexes based upon specific RNA binding proteins and the identification of RNAs dissociated with the mRNP complex. RAS™ can be performed [symbol] in at least three ways; A) In vivo RAS™ using antibodies against the native endogenous RNA binding protein, B) In vivo RAS™ using epitope-tagged RNA binding protein and an antibody against the epitope, C) In vitro RAS™ using purified recombinant RNA binding protein and ce [symbol] extracts or purified RNA.

FIG. 4 is a schematic of using RIBOTRAP™ and RAS™ for polypyrimidine tract binding protein (PTB, or RBP-1). A ribonomic cluster is isolated from cell extracts using antibodies specific for RBP-1. RNA extracted from this cluster is compared to total RNA by global microarray analysis.

FIG. 5 is a schematic overview of an embodiment of a target discovery process using RNA binding proteins and mRNP complexes.

FIG. 6 is a schematic overview of an exemplary data flow for analyzing and interpreting microarray results from comparative RNA binding protein expression and/or mRN [symbol] complexes for identifying tissue or disease-specific RNA binding proteins, mRNAs, and genes

[symbol]

FIG. 7 is a Western blot illustrating the in vitro RIBOTRAP™, verifying that PTB fr [symbol] INS-1 cell lysates specifically binds the oligonucleotides encoding a portion the 3′UTR of preproinsulin and not oligonucleotides encoding a control oligonucleotide. In addition, glucose [symbol] stimulates an acute and transient increase in PTB binding. Lanes 1 and 2: total cell lysate; Lane [symbol] 3 and 4: control oligonucleotides; Lanes 5 and 6: 5′ UTR oligonucleotides; Lanes 7 and 8: 3′UTR oligonucleotides.

FIG. 8 illustrates a proposed model of glucose-regulated RNA binding protein binding to preproinsulin mRNA and regulation of glucose-induced preproinsulin translation by RNA binding proteins. Sp, signal peptides; B, C, A, coding regions for various peptide chains of processed insulin.

FIG. 9 is a schematic overview of target discovery in primary adipocytes.

FIG. 10 is a list of RNA binding protein genes whose expression is differentially regulated (2-fold or more) during differentiation of human pre-adipocytes to adipocytes. RNA was isolated from lean patients pre-adipocytes and RNA from lean patients differentiated adipocytes.

FIG. 11 is a list of RNA binding protein genes that are up-regulated 2-fold or more during differentiation of adipocytes from obese patients.

FIG. 12 is a list of RNA binding proteins that are differentially expressed (2-fold or more) in human adipocytes treated with BRL-37433. RNA was isolated from human adipocyte [symbol] prepared from lean (non-obese) patients that were either left untreated or with the β-3 adrenergic [symbol] agonist, BRL-37344 (1 μM).

FIG. 13 is a list of RNA binding proteins that are differentially expressed (2-fold or more) in human adipocytes treated with insulin. RNA was isolated from human adipocytes prepared from lean (non-obese) patients that were either left untreated or with insulin (100 nM).

FIG. 14 is a list of RNA binding proteins that are differentially regulated by glucose in INS-1 cells.

FIG. 15 is a list of RNA binding protein genes differentially expressed in HepG2 cells treated with bezafibrate.

FIG. 16 is a list of RNA binding protein genes differentially expressed in HepG2 cells treated with Wyeth 14643.

FIG. 17 is a list of RNA binding protein genes differentially expressed in HepG2 cells treated with troglitazone.

FIG. 18 is a list of RNA binding protein genes differentially expressed in HepG2 cells treated with MCC-555.

FIG. 19 is a list of RNA binding protein genes differentially expressed in HepG2 cells treated with ciglitazone.

FIG. 20 is a list of RNA binding protein genes differentially expressed in HepG2 cells treated with 2-bromohexadecanoic acid (2-BHDA).

FIG. 21 is a list of RNA binding protein genes differentially expressed in HepG2 cells treated with prostaglandin J2 (PJ2).

FIG. 22 is a list of RNA binding protein genes differentially expressed in HepG2 cells treated with perfluorooctanoic acid (PFOA).

FIG. 23 is a list of genes identified in an in vitro RAS™ analysis of GST-PTB. These genes and their encoded proteins represent candidate therapeutic targets of cellular pathways involved in glucose and lipid metabolism, insulin action, insulin resistance, diabetes and obesity.

FIG. 24 shows examples of target validation using RNAi mediated gene silencing followed by an assay to determine glucose-stimulated insulin secretion. FIG. 24A shows effects [symbol] of RNAi mediated gene silencing of PTB on insulin secretion. FIG. 24B shows effect of RNA mediated gene silencing of three ion channels contained within the PTB ribonomic cluster. FIG. 24C shows the effect of RNAi mediated gene silencing of IonCh4 or CNCG on insulin secretion.

FIG. 25 is a schematic for the regulatory mechanisms of insulin secretion in pancreatic beta cells. Proteins that are shown in bold print are present on the PTB cluster.

FIG. 26A shows an immunoblot probed with a PTB monoclonal antibody showing PT binding to a preproinsulin 3′UTR oligonucleotide after cells were grown in various amounts of glucose. FIG. 26B is a bar graph depicting the data from FIG. 26A.

FIG. 27 is a refined list of candidate therapeutic targets obtained from the PTB ribonomic cluster and is organized into druggable target classes.

FIG. 28 shows the effect of PTB inhibition by RNAi on the expression of PTB, preproinsulin as well as nine additional genes found within the PTB-cluster: CACNA1s, CACNA2D1, Casr, Clc3, KCNJ6, and Loc245960. As indicated in FIG. 28A, there was an 80% reduction in PTB mRNA expression, confirming the action of the PTB specific RNAi. Expression of some of the other genes was also downregulated to varying degrees. FIG. 28B shows genes whose expression was up-regulated as a result of PTB knockdown, which includes preproinsulin mRNA, which is up-regulated 3-fold.

### DETAILED DESCRIPTION

The invention provides methods for mining and characterizing the cellular ribonome in cells that participate in regulatory pathways, such as, for example, insulin action, insulin production and secretion, glucose metabolism, and lipid metabolism. The resulting ribonomic profile provides a subset of genes, and the mRNAs and proteins they encode, as potential therapeutic targets for altering or regulating those pathways.

Methods of the invention comprise identifying and measuring mRNP complex components. Differentially expressed mRNP complex components are potential therapeutic targets, and are useful for assessing the efficacy or toxicity of potential therapeutics. The invention also provides methods for identifying and characterizing structurally and/or functionally related gene products, and for elucidating features of biological pathways or other cellular functions. The identified mRNP complex components are also useful for diagnosing, monitoring, and assessing the metabolic or disease state of a cell or organism.

Generally, mRNP complex components include, but are not limited to, at least one RNA [symbol] binding protein, and at least one associated or bound mRNA. The mRNP complex may also include at least one associated or bound protein (i.e., an mRNP complex-associated protein) or other associated or bound molecules (e.g., carbohydrates, lipids, vitamins, etc.). A component associates with an mRNP complex if it binds or otherwise attaches to the mRNP complex with Kd of about 10⁻⁵ to about 10⁻¹². In an embodiment, the component associates with the complex with a Kd of about 10⁻⁷ to about 10⁻⁹. In another embodiment, the component associates with the [symbol] complex with a Kd of about 10⁻⁸ to about 10⁻⁹.

By isolating an mRNP complex from a cell and, preferably, identifying the components of the mRNP complex and the gene precursors and gene products of those components, a ribonomic profile is generated. The associated or bound RNAs are categorized into subsets based on their association with a particular RNA binding protein, mRNP complex-associated protein, mRNA, or other common structural or functional feature. Ribonomic profiles differ from cell sample to cell sample, depending on a variety of factors including, but not limited to, the species or tissue type of the cell, the developmental stage of the cell, the differentiation stat [symbol] of the cell (e.g., malignant) the pathogenicity of the cell (e.g., if the cell is infected, is expressing [symbol] a deleterious gene, is lacking a particular gene, is not expressing or is underexpressing a particular gene, or is overexpressing a particular gene), the various conditions or agents affecting [symbol] the cell (e.g., treatment with a therapeutic, environmental, apoptotic or stress state, and the specific ligands used to isolate the mRNP complexes, as well as other factors known to practitioners in the art. The profile therefore provides a footprint of the gene expression of the cell samples that can be used to identify therapeutic targets and to elucidate components of cellular pathways in normal or disease cells.

Identification and Isolation of mRNP Complexes and RNA Binding Proteins

RNA binding proteins involved in a particular pattern, pathway, or disease state, are identified by a variety of methods in the art. For example, the expression of RNA binding proteins that are differentially expressed between normal and disease samples or normal and agent-treated samples can be assessed using methods such as Northern blot, Quantitative Real Time Polymerase Chain Reaction (QRT-PCR), Western blot, microassay analysis, Serial Analysis of Gene Expression (SAGE), cloning and sequencing, or other methods known to the skilled artisan.

Alternatively, differentially expressed RNA binding proteins can be efficiently identified using either a microarray such as a RIBOCHIP™. A RIBOCHIP™ (MWG Biotech, High Point, N.C.) is a microarray that is used to assay the expression level for a large number of RNA binding proteins. The RIBOCHIP™ contains 50-mer oligonucleotides representing genes, the protein products of which are reported to have RNA binding properties or to contain RNA binding motifs. These genes include those identified in FIGS. 10-22, and described in Examples 1-5. Also included on the array are control features (a total of 17) that provide information on specificity, labeling and hybridization efficiency, sensitivity and normalization between experiments.

In an embodiment, cell samples containing mRNAs encoding RNA binding proteins are used to probe a microarray containing nucleic acid sequences encoding at least a portion of a number of RNA binding proteins, in order to detect and/or measure the expression of RNA binding proteins in the sample. Sample mRNAs are prepared from cell lines or tissues from control, agent-treated, normal, or diseased states, for example. The agent may be any agent that alters gene expression, for example, glucose, insulin, a beta-adrenergic agonist (e.g., BRL-37433), insulin-like growth factor-1 (IGF-1), glucagon-like peptide-1 (GLP-1), fatty acid, peroxisome proliferator activated receptor (PPAR) ligands (e.g., thiazolidinediones, fibrates, halogenated fatty acids, and tyrosine derivatives), insulin-like growth factor-2 (IGF-2). The agent may also be an RNAi that inhibits an RNA binding protein, an enhancer of RNA binding protein expression, a nucleic acid, a hormone, an antibody, an antibody fragment, an antigen, a cytokine, a growth factor, a pharmacological agent (e.g., chemotherapeutic, carcinogenic), a chemical composition, a protein, a peptide, and/or a small molecule. The mRNA samples are amplified if necessary, and processed for microarray hybridization.

Microarray analysis enables RNA binding protein genes with unique or differential expression profiles to be quickly identified and clustered into functional or structural categories from among the thousand genes profiled in a single experiment. Several specific examples of microarray analysis and lists of relevant RNA binding protein genes and encoded proteins that are differentially expressed are provided in Examples 3-5. These differentially expressed RNA binding proteins genes are involved in, for example, obesity, adipocyte differentiation, insulin action, insulin production and secretion, diabetes, mechanisms of action of PPAR ligands, insulin resistance, glucose metabolism, lipid metabolism, hypoglycemia, glucotoxicity, lipid toxicity, insulin resistance, hyperlipidemia, and lipodystrophy.

Pancreatic beta cell lines or freshly prepared islets are physiologically relevant ex vivo model systems for examining glucose-responsiveness and endocrine pancreas functions. To identify RNA binding proteins that undergo changes in expression, cells are incubated under conditions of low (e.g., 3 mM) or high (e.g., 15 mM) glucose for various periods of time. Total mRNA is prepared according to standard methods. In some cases where samples are limiting, it may be necessary to amplify the mRNA according to standard RT-PCR methods or kits such as the RIBOAMP™ kit (Arcturus, Mountain View, Calif.). Differentially expressed RNA binding protein genes identified by microarray analysis represent RNA binding proteins whose expression is regulated by glucose.

In another embodiment, mRNA and protein levels of RNA binding proteins are determined in cell lines such as the alpha cell line, α-TC1.6, the rat pancreatic beta cell line INS [symbol] 1 cells (Beta-gene, Dallas, Tex.), and mouse pancreatic beta cell line MIN-6 cells, for example, to [symbol] characterize the mechanisms of gene expression that are particular to that cell type. For example, α-TC1.6 cells express Nkx6.1 mRNA but do not express Nkx6.1 protein. In contrast, INS-1 cells express both Nkx6.1 mRNA and Nkx6.1 protein. Current evidence supports a role for RNA binding proteins in this restrictive expression during islet development.

In another embodiment, human preadipocytes or adipocytes are isolated from lean or obese patients and differential expression of RNA binding proteins is obtained by microarray analysis. These RNA binding protein genes and their gene products function in adipocyte differentiation, adipocyte function, insulin action, insulin resistance, obesity and glucose and lipid metabolic pathways, for example.

RIBOTRAP™

Whereas microarray analysis allows for the simultaneous analysis of the expression of RNA binding proteins, RIBOTRAP™ combines a biochemical and molecular biological approach for isolating, or “trapping”, an unknown RNA binding protein or set of RNA binding proteins that interact with an nucleic acid of interest. This involves several different approaches, including the use of 1) affinity-labeled or epitope-tagged RNA binding elements as affinity reagents for in vitro isolation of RNA binding proteins and 2) expression or transformation of an affinity-labeled or epitope-tagged mRNA in cell culture models for isolation of RNA binding proteins bound to the tagged mRNA in vivo. RIBOTRAP™ is useful when it is necessary to first identify an RNA binding protein on a specific mRNA. RIBOTRAP™ methods are described in detail in Example 2.

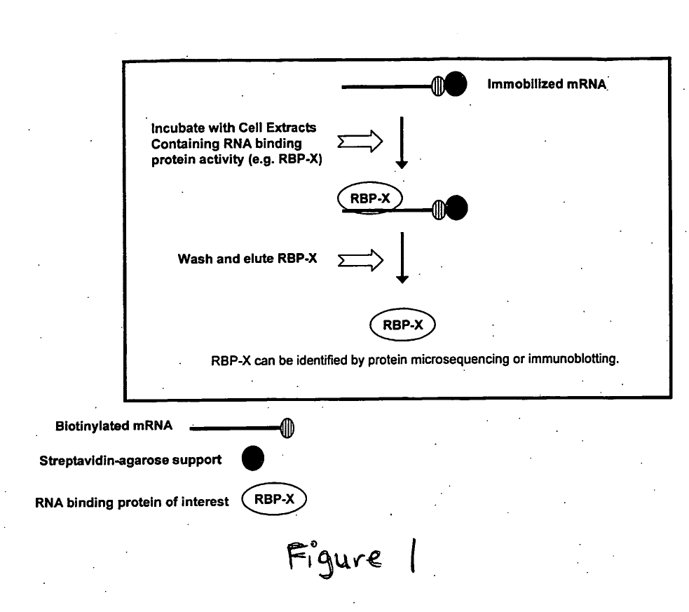

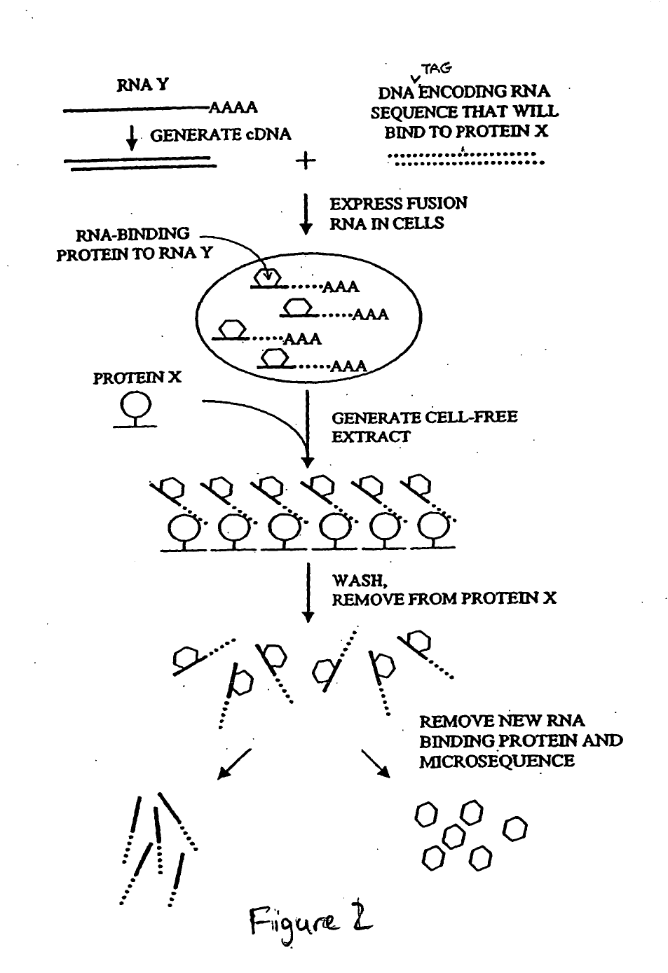

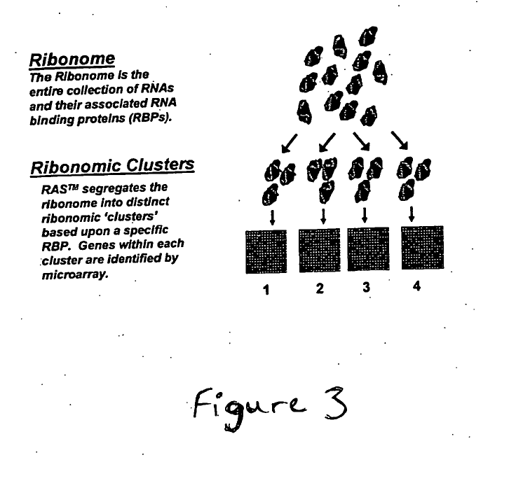

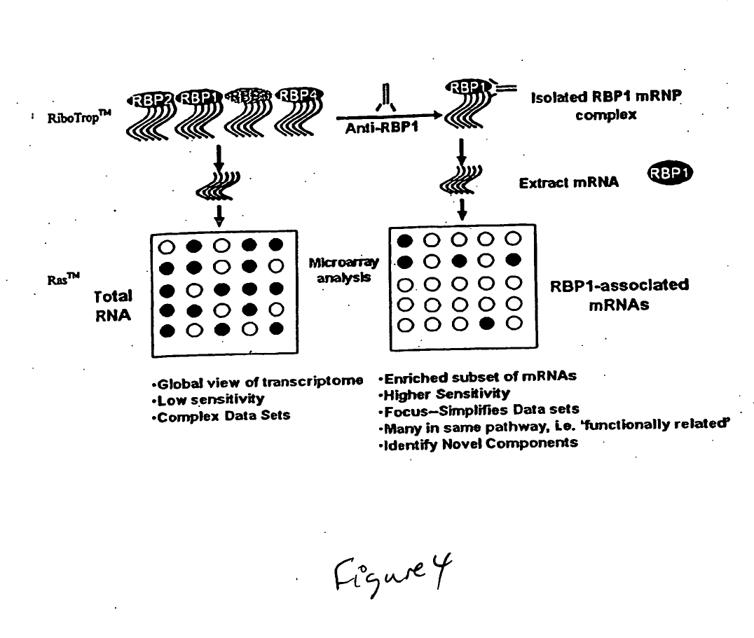

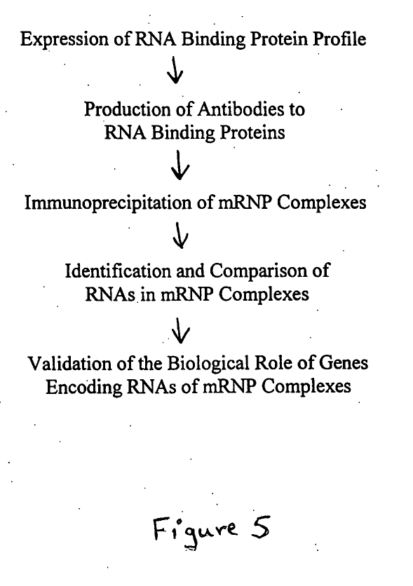

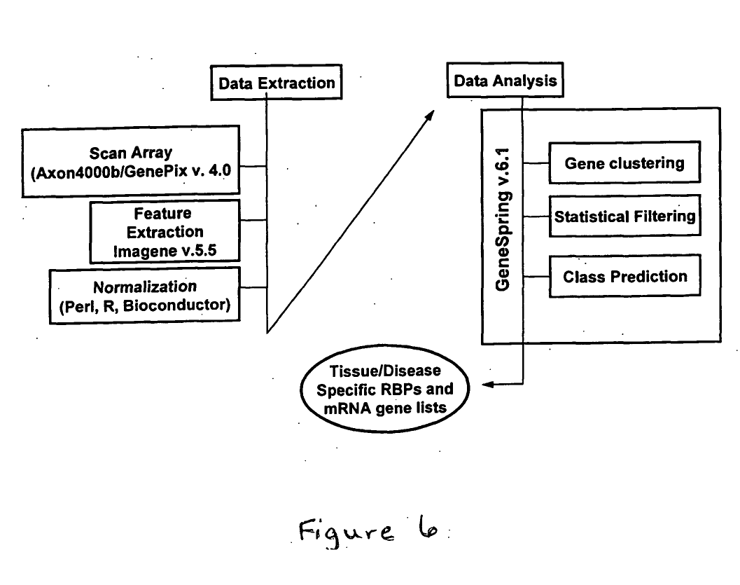

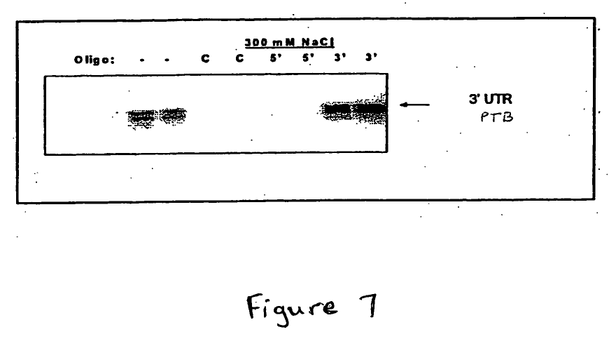

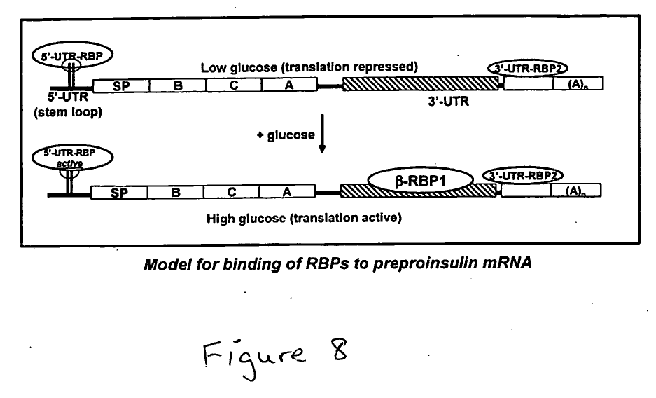

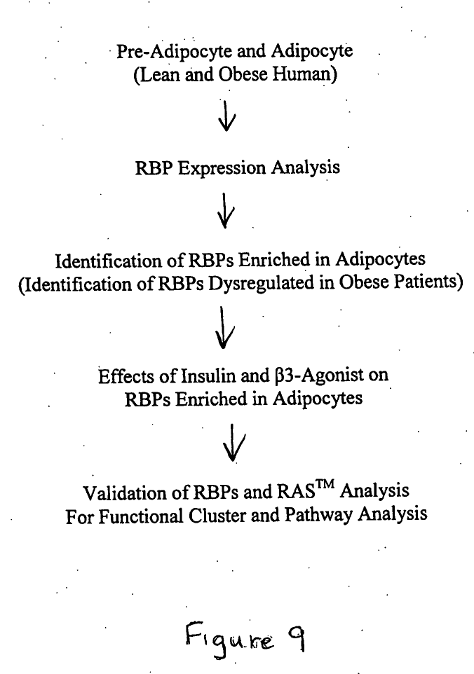

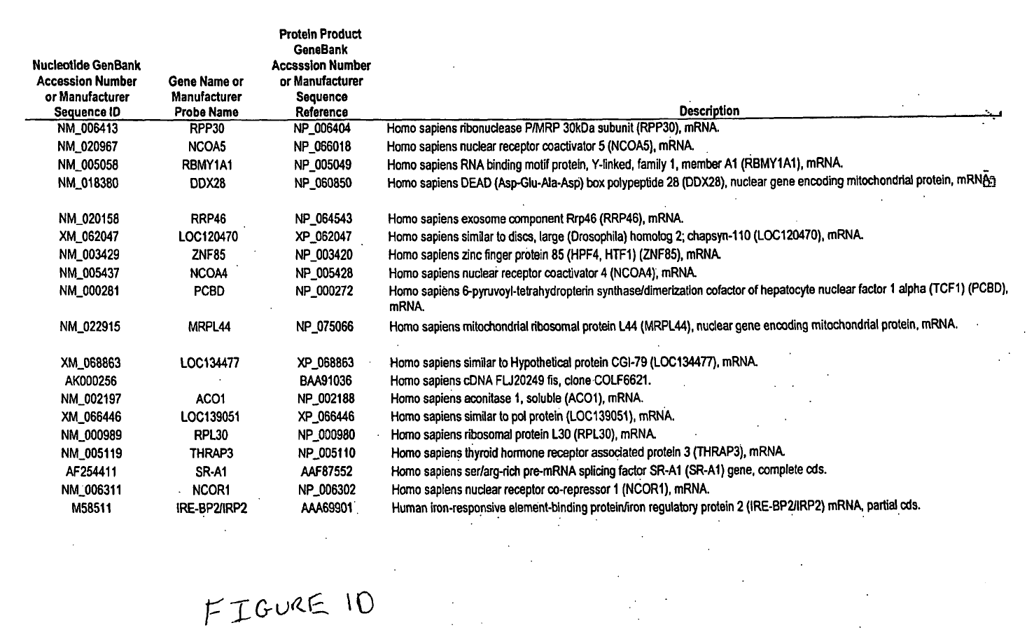

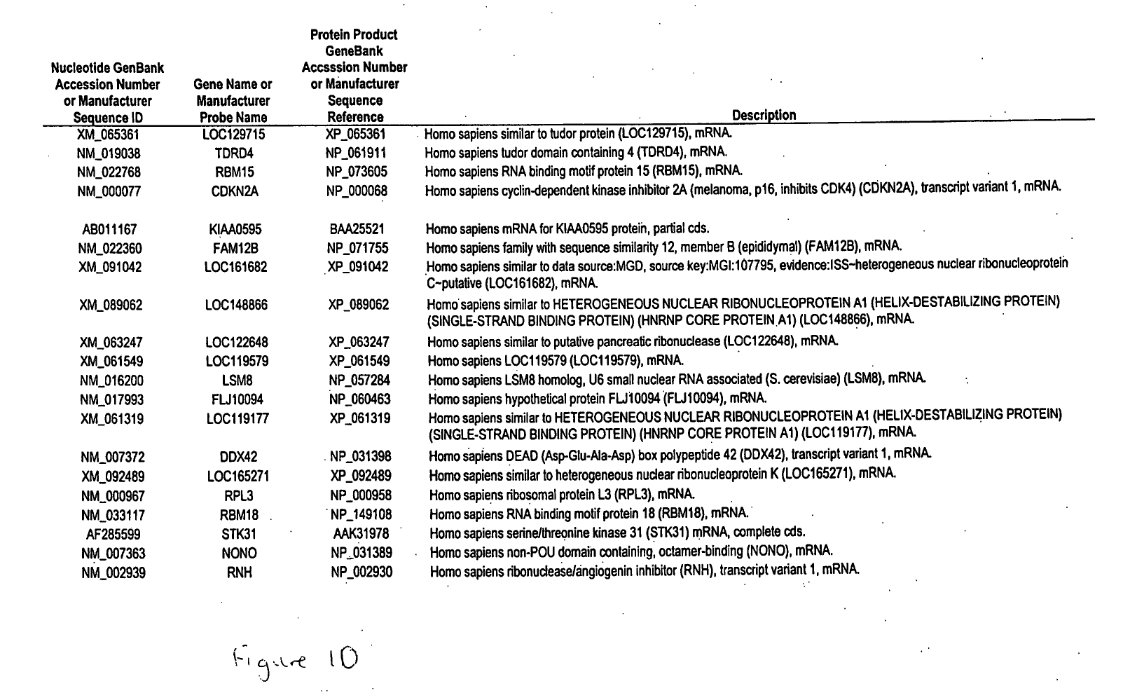

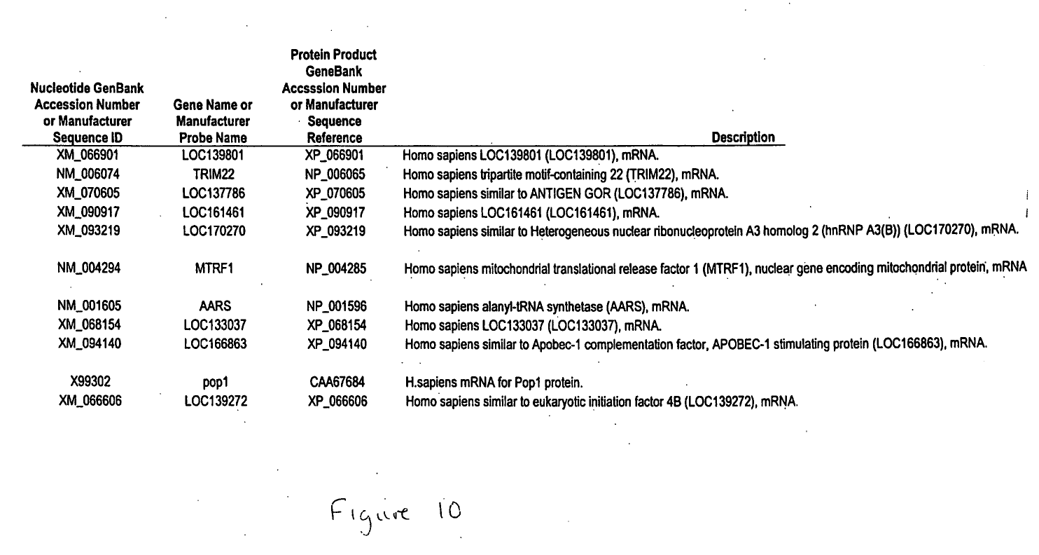

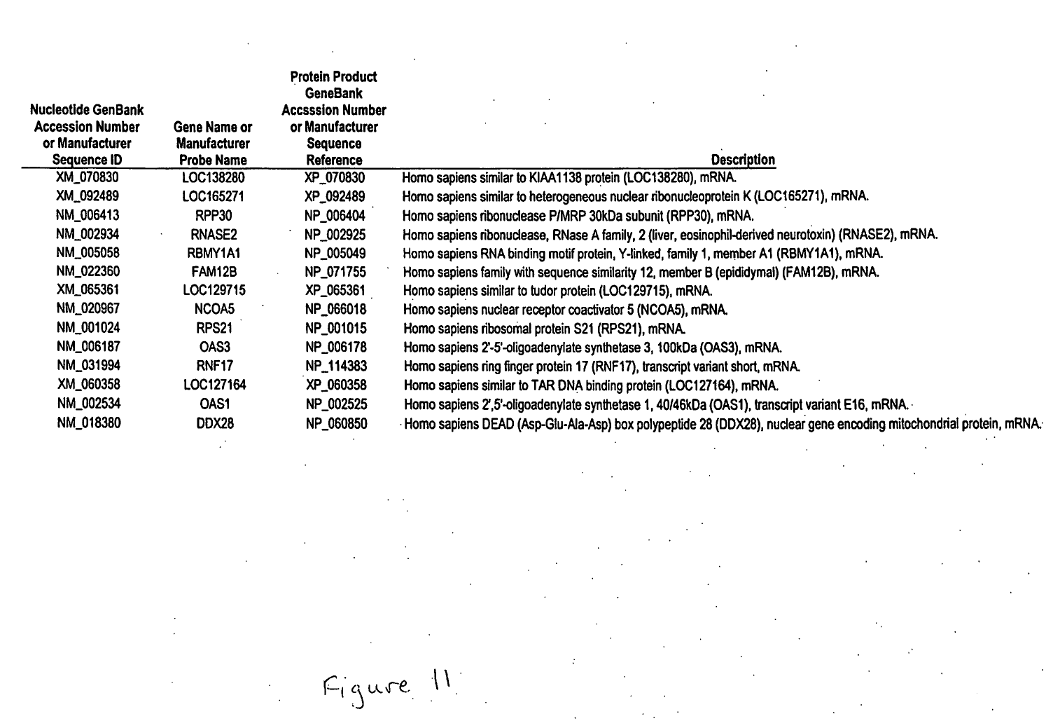

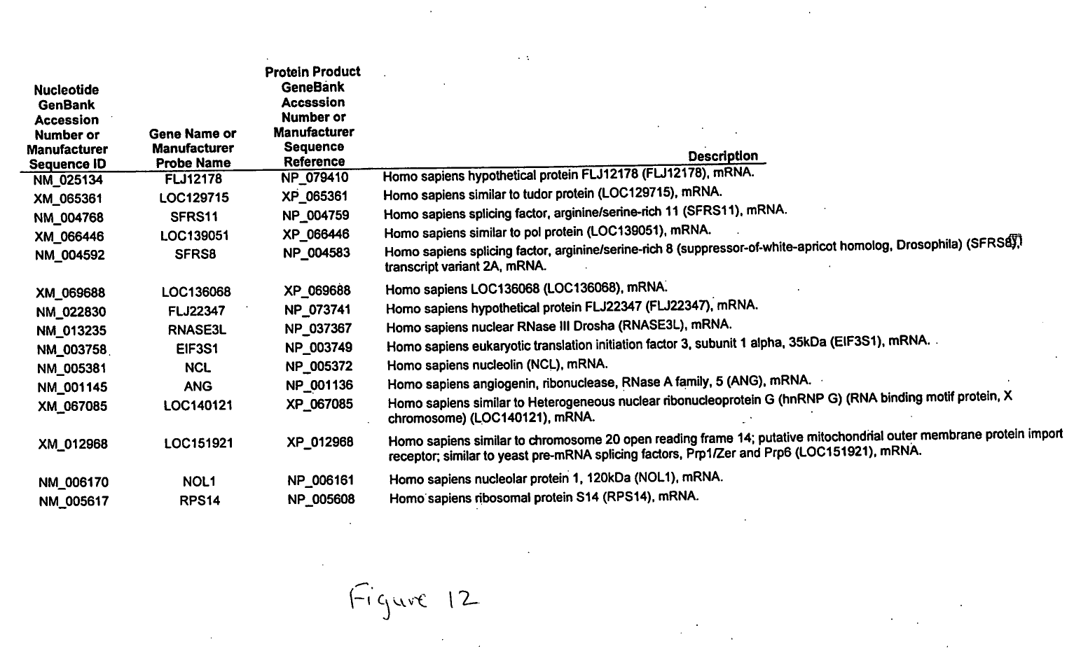

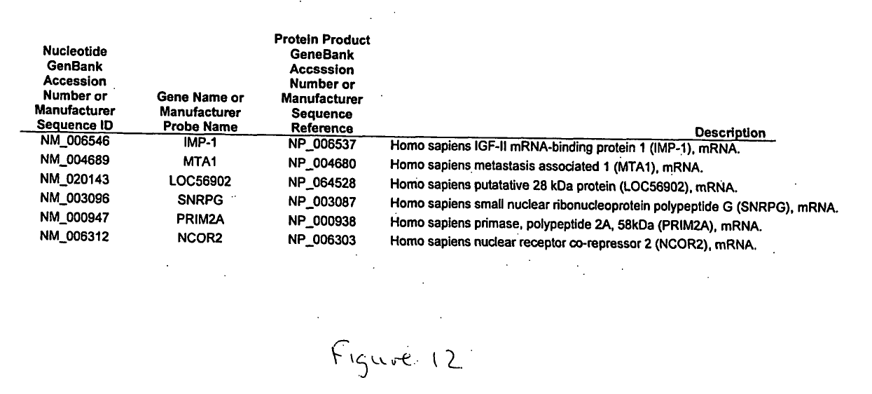

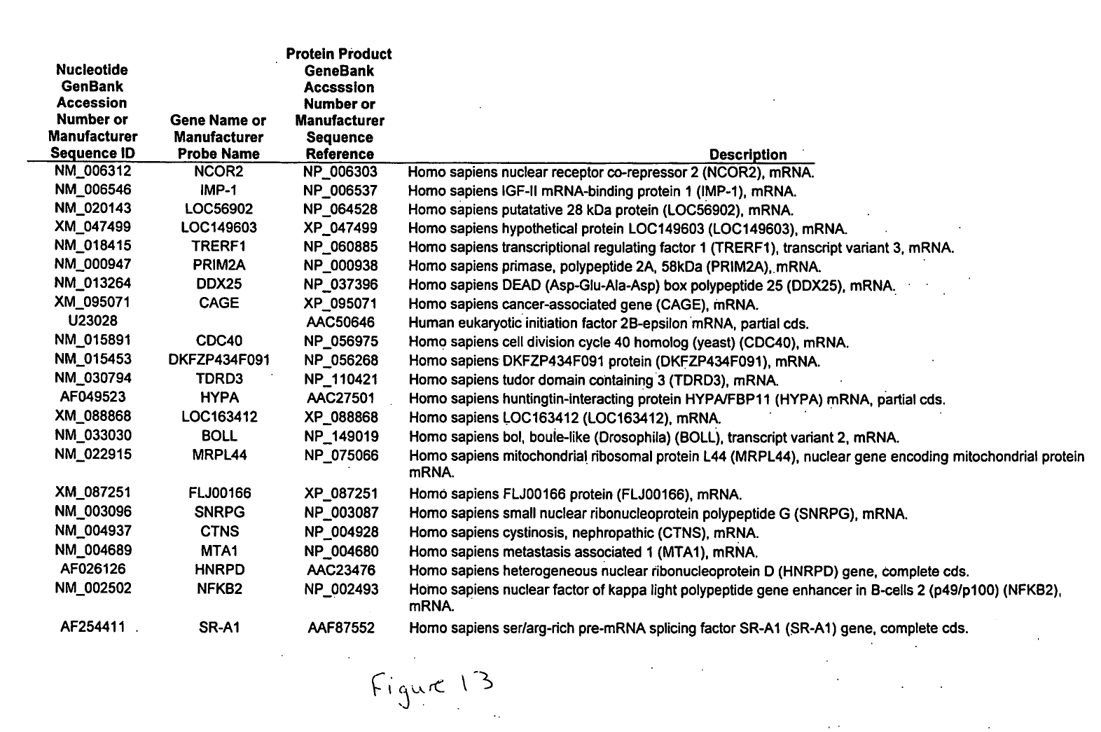

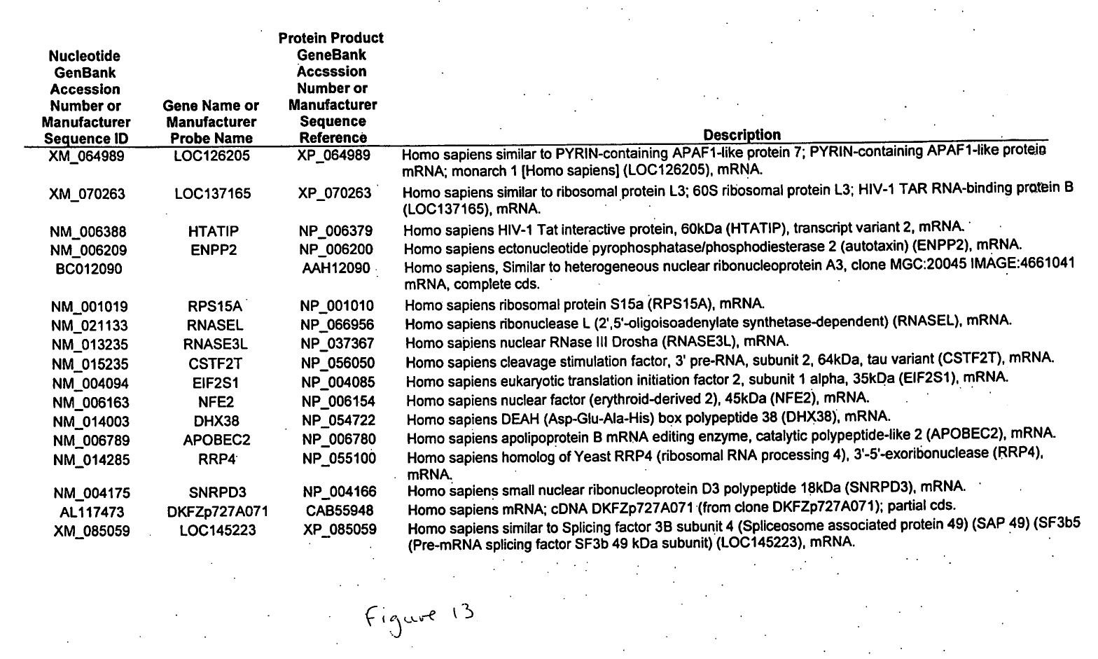

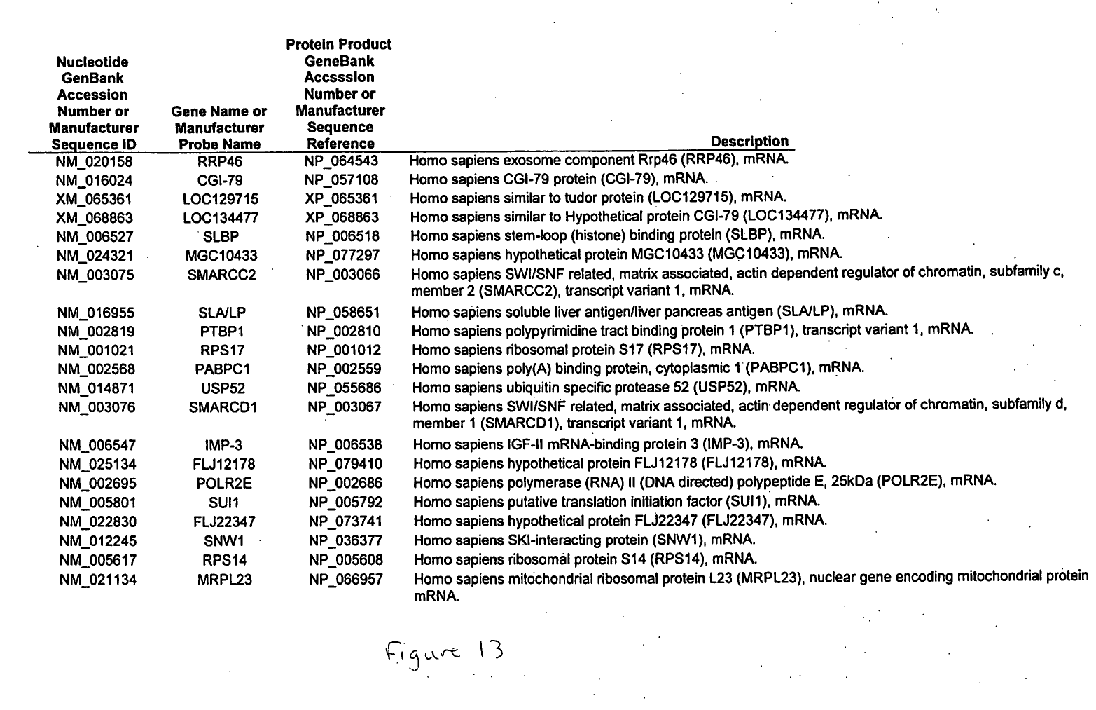

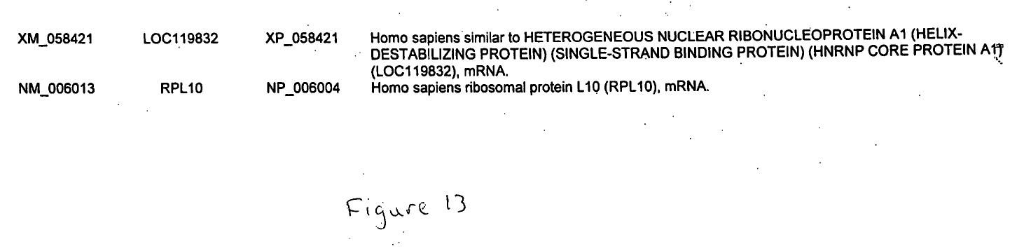

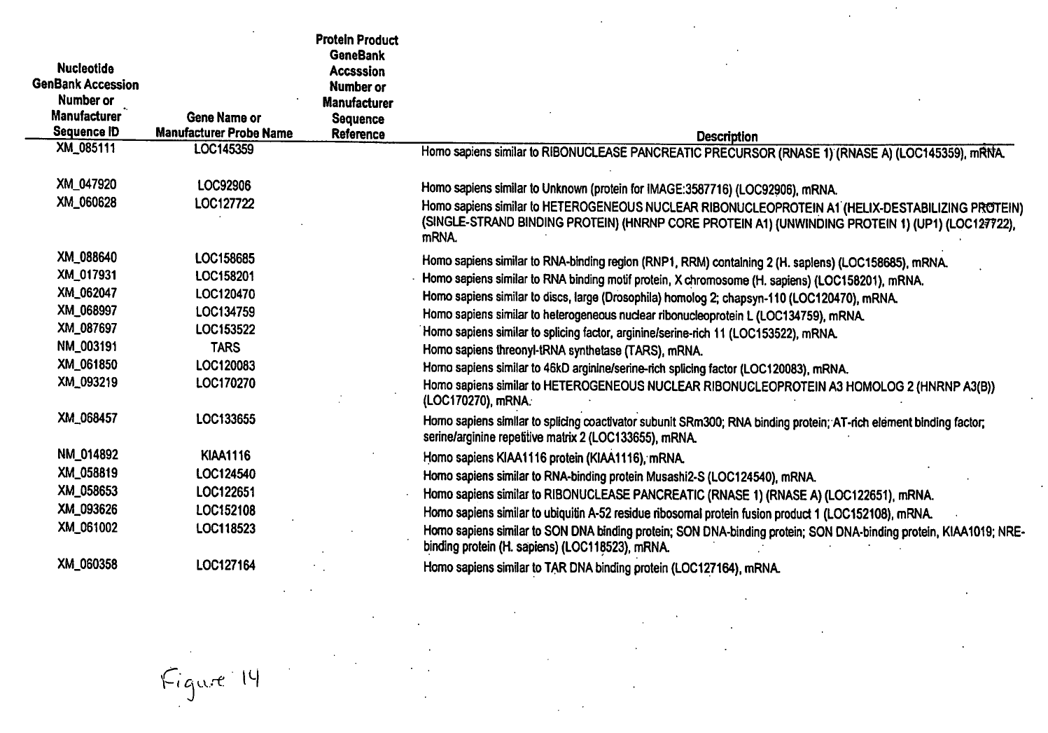

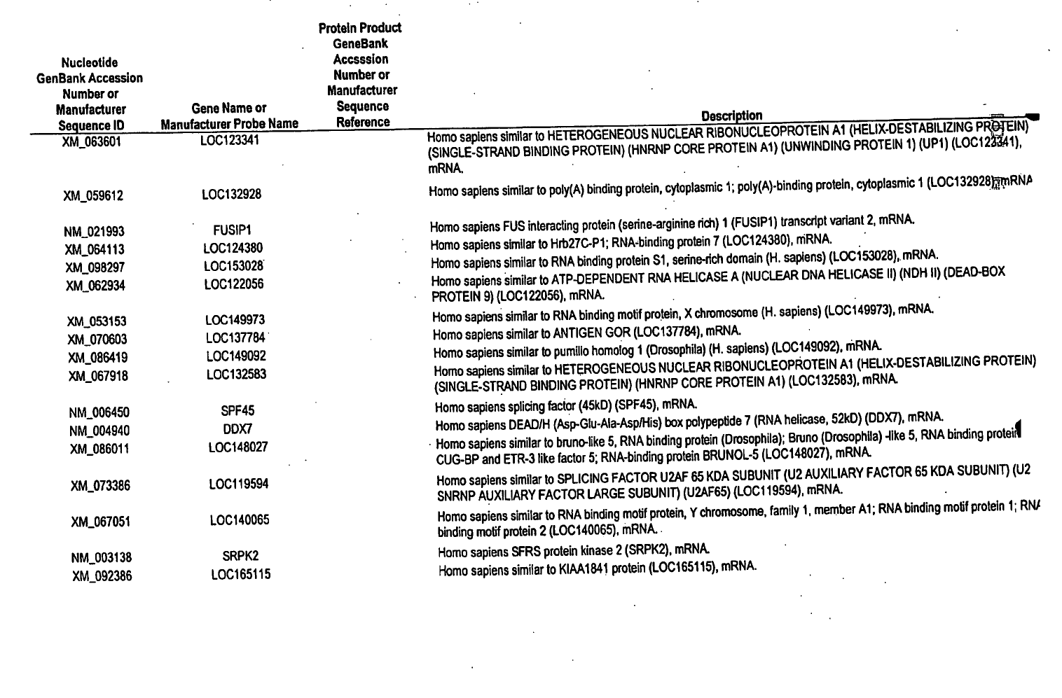

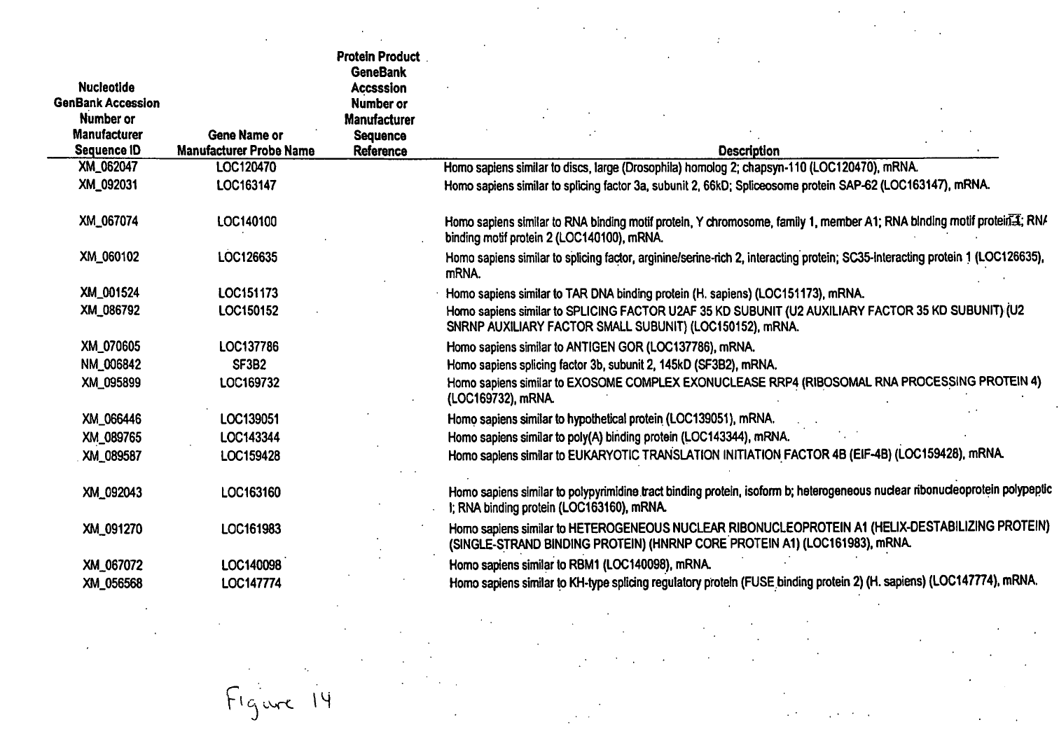

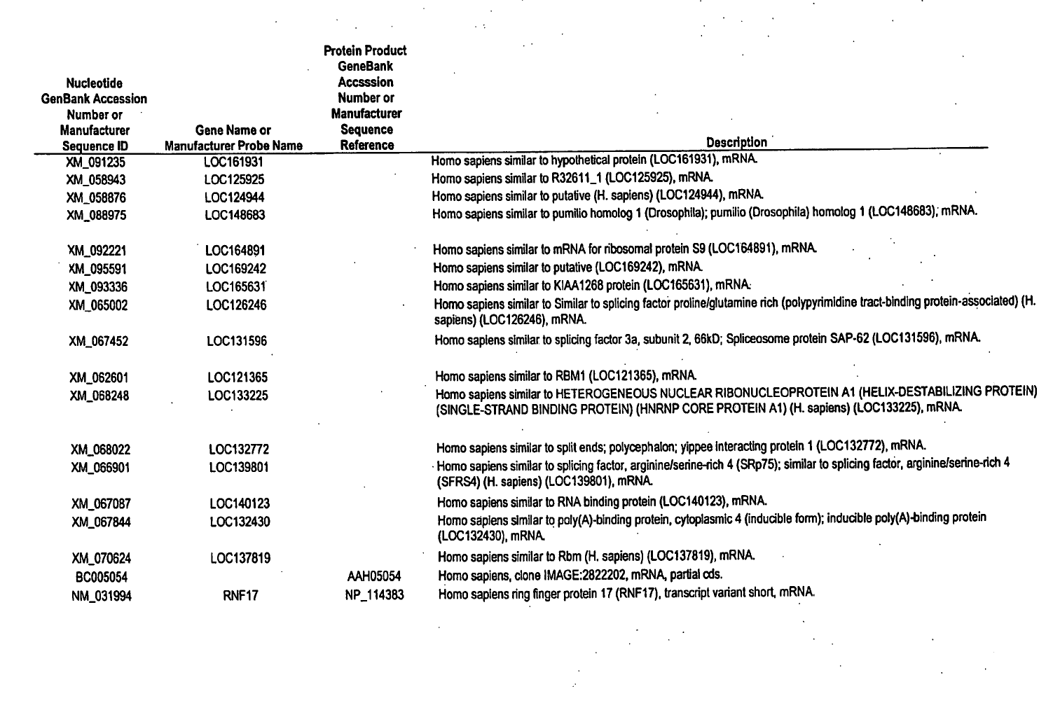

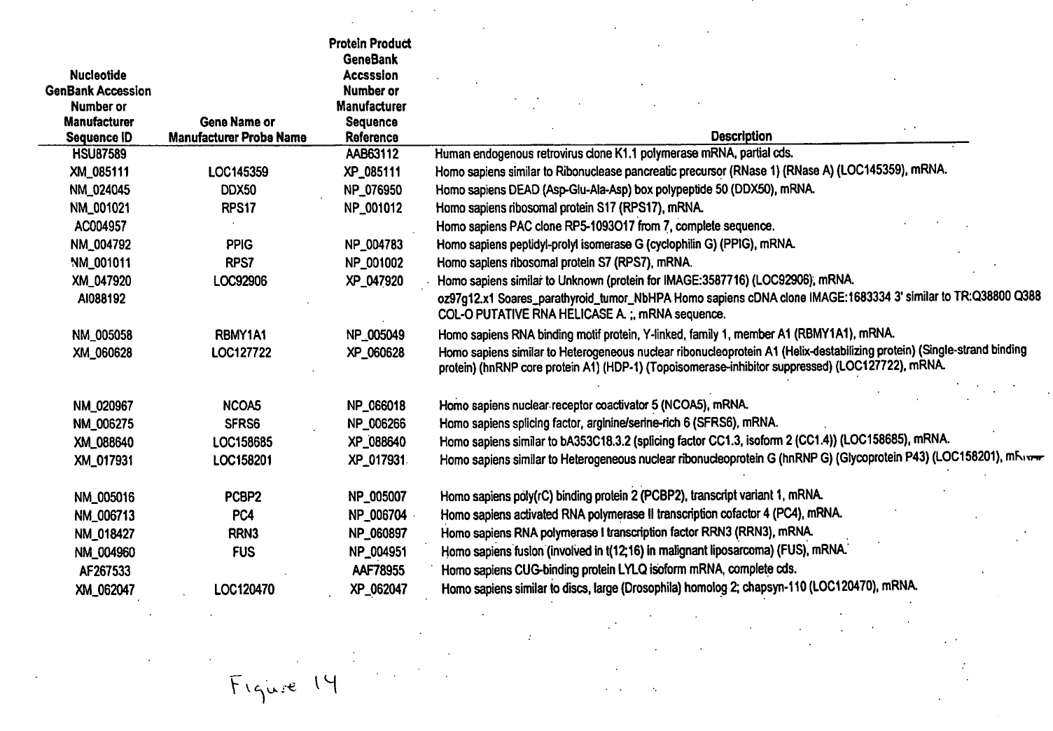

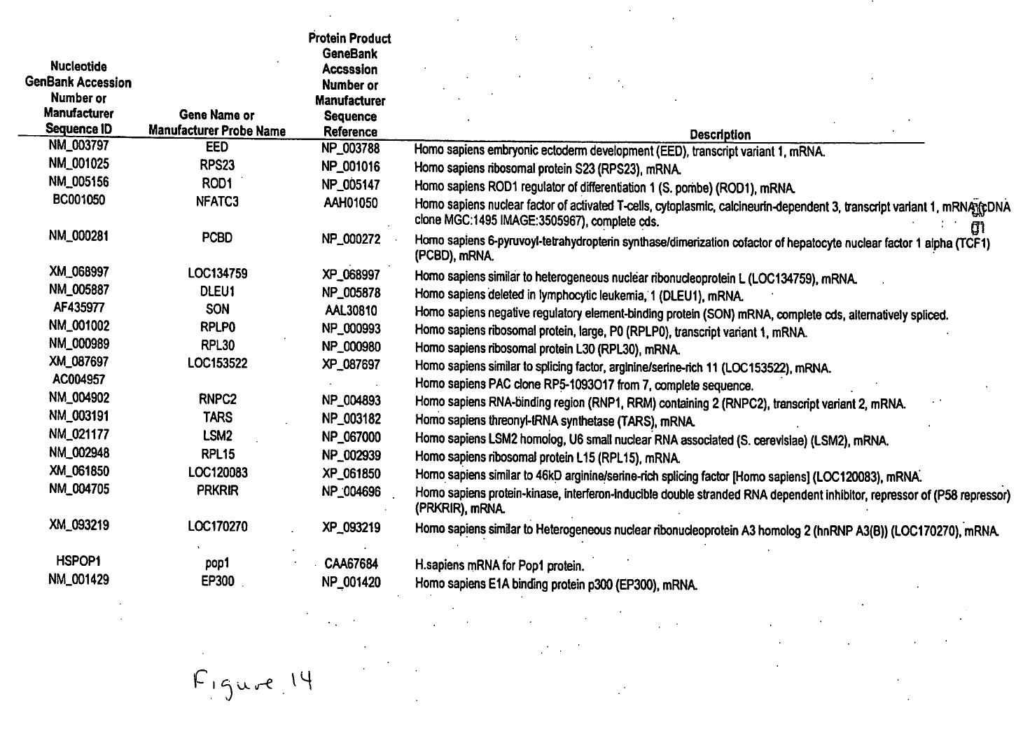

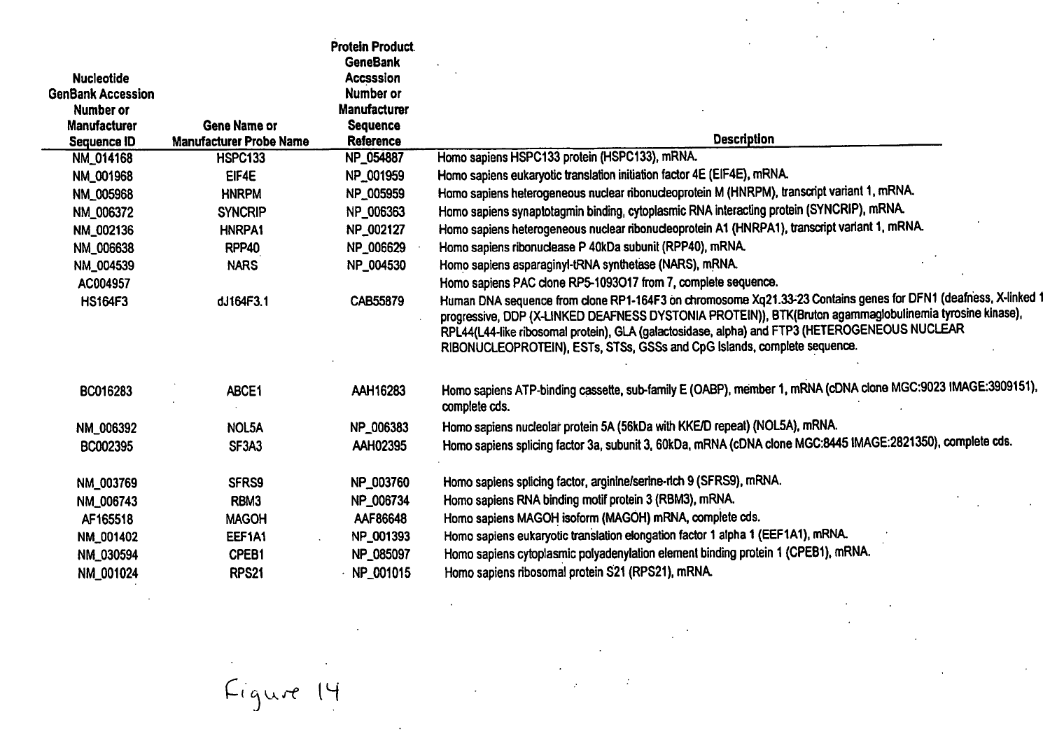

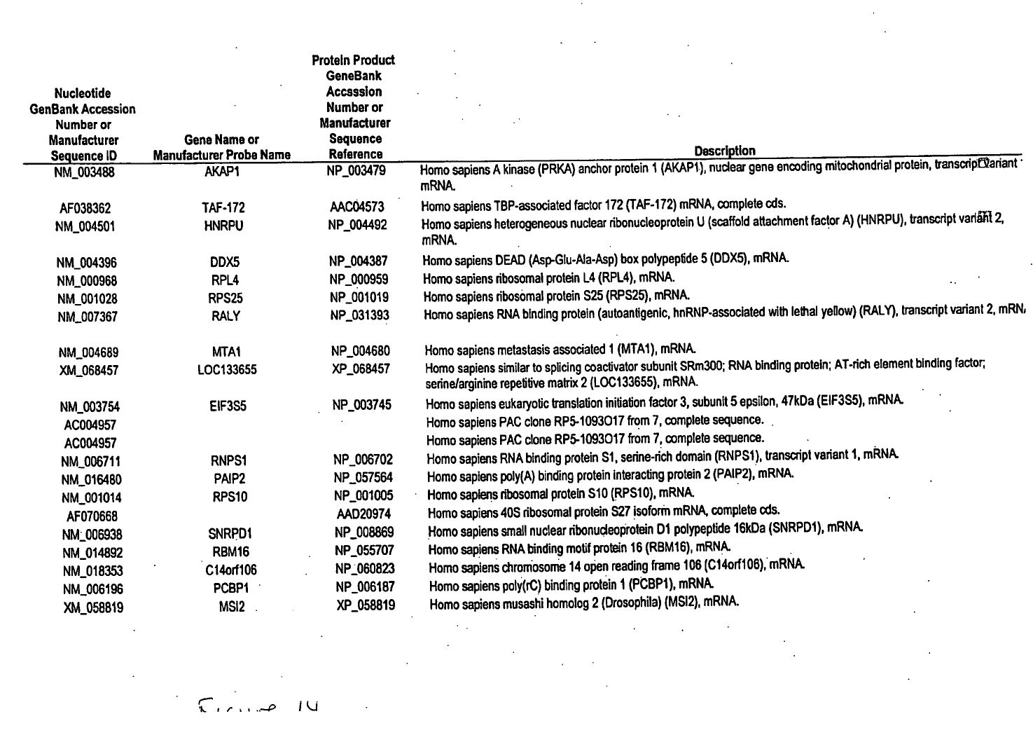

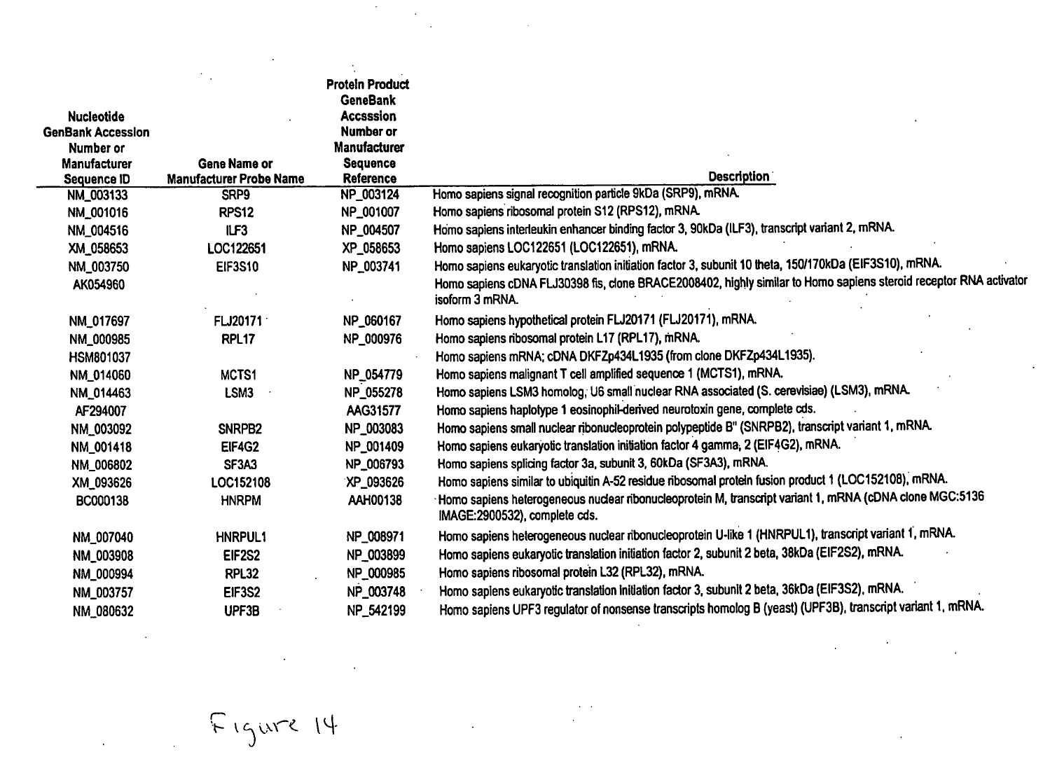

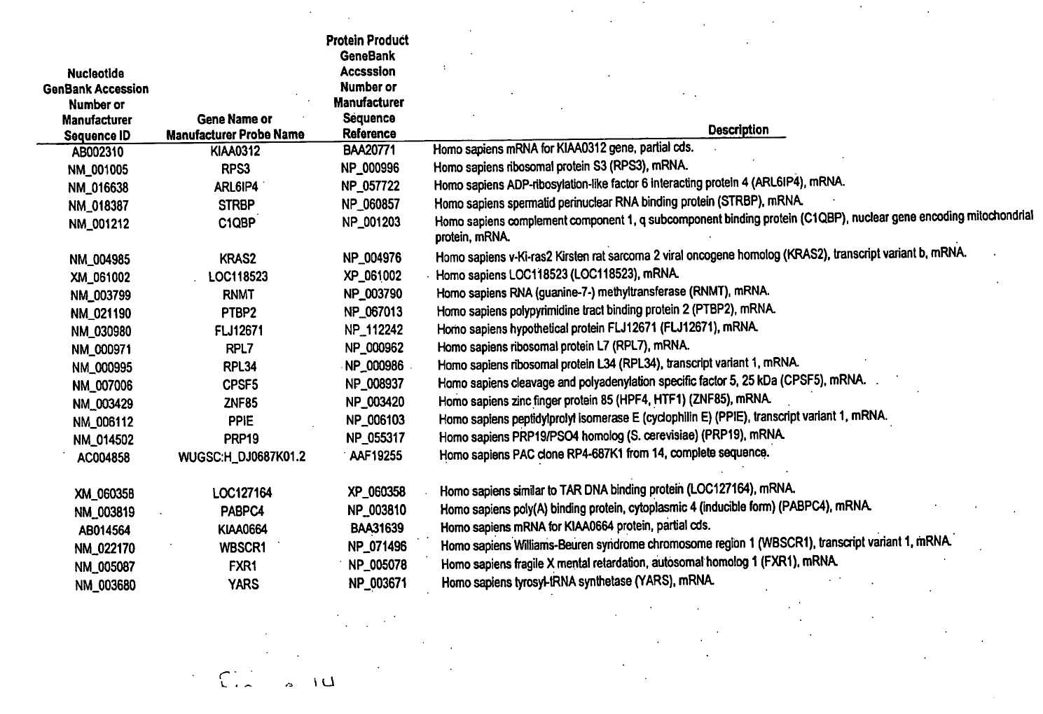

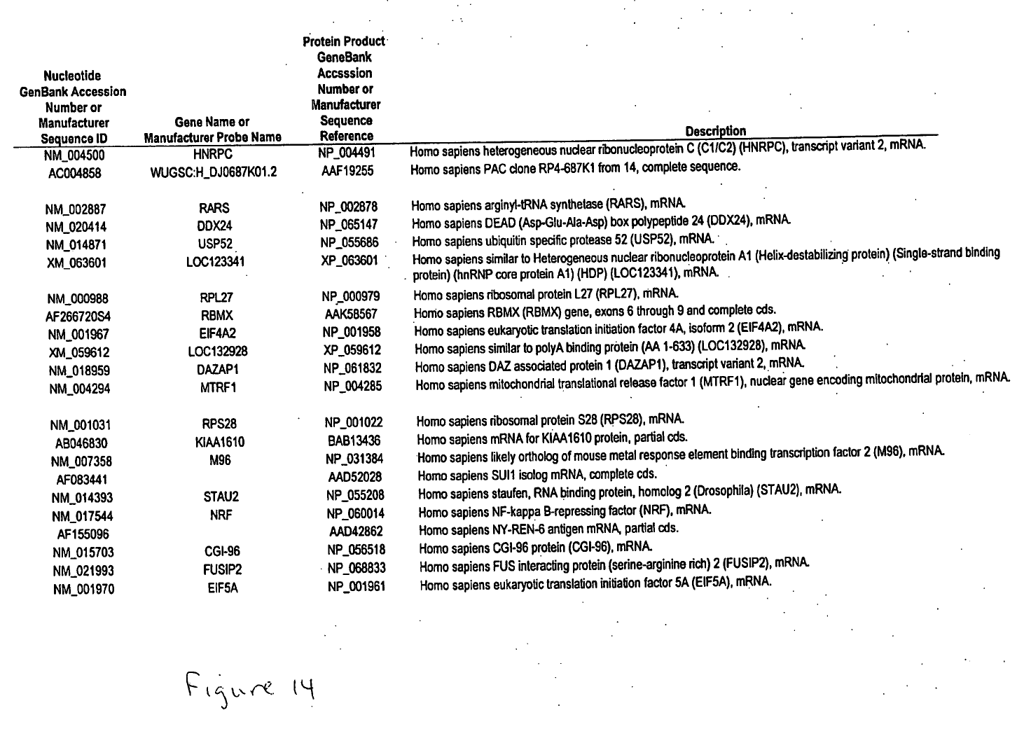

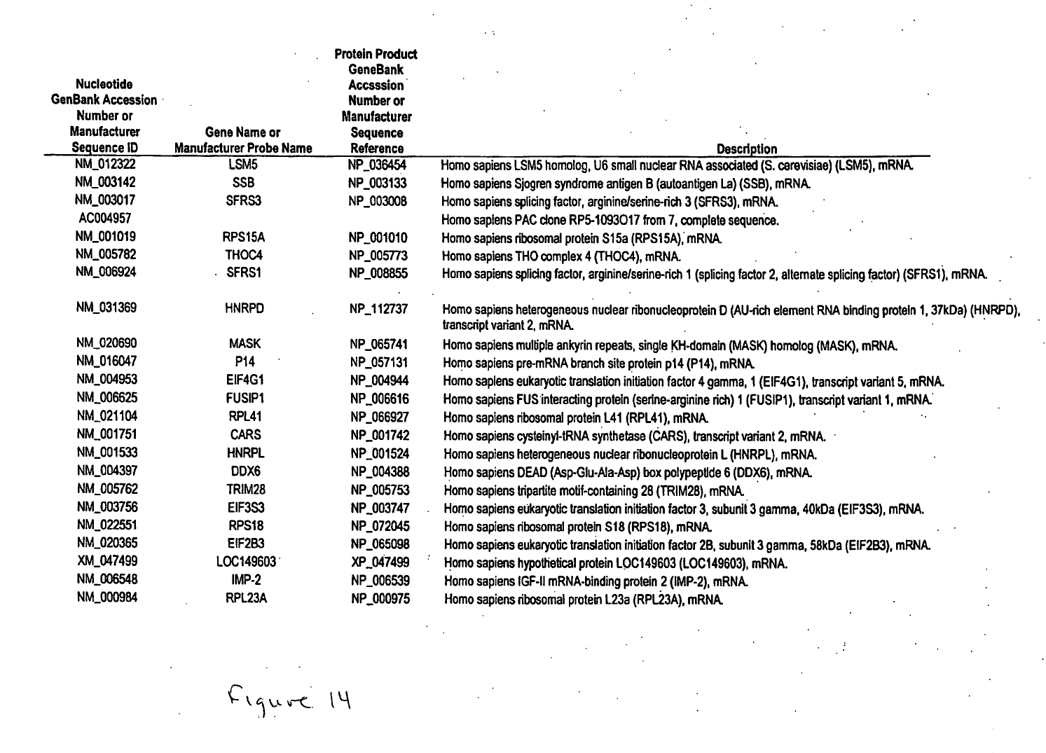

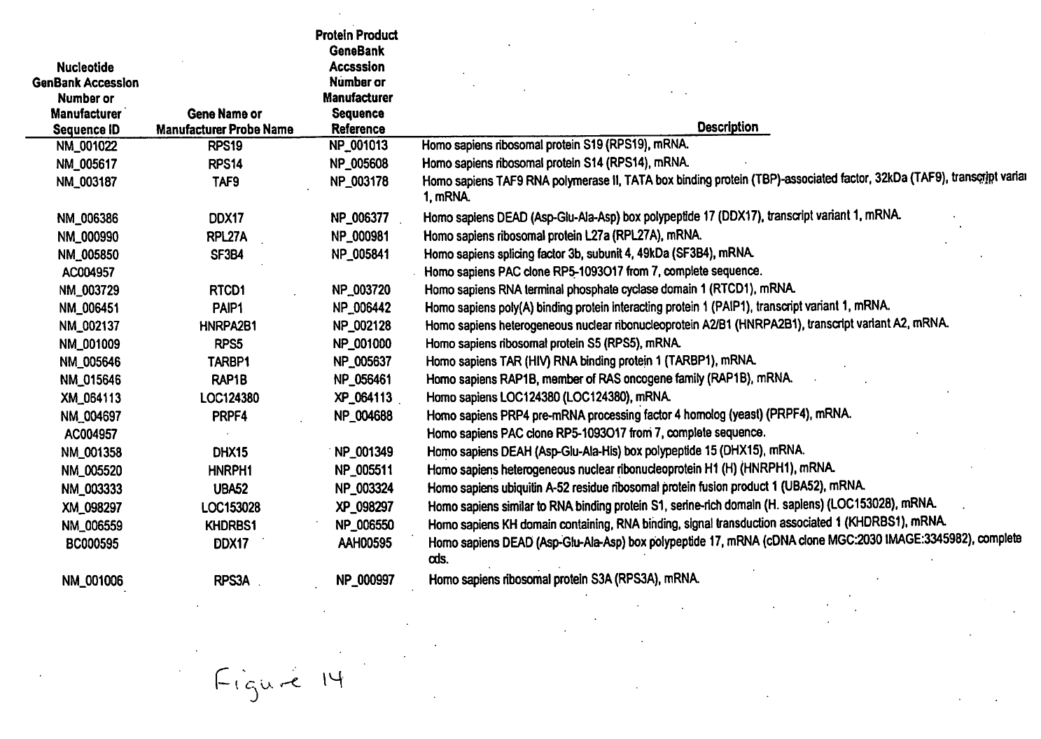

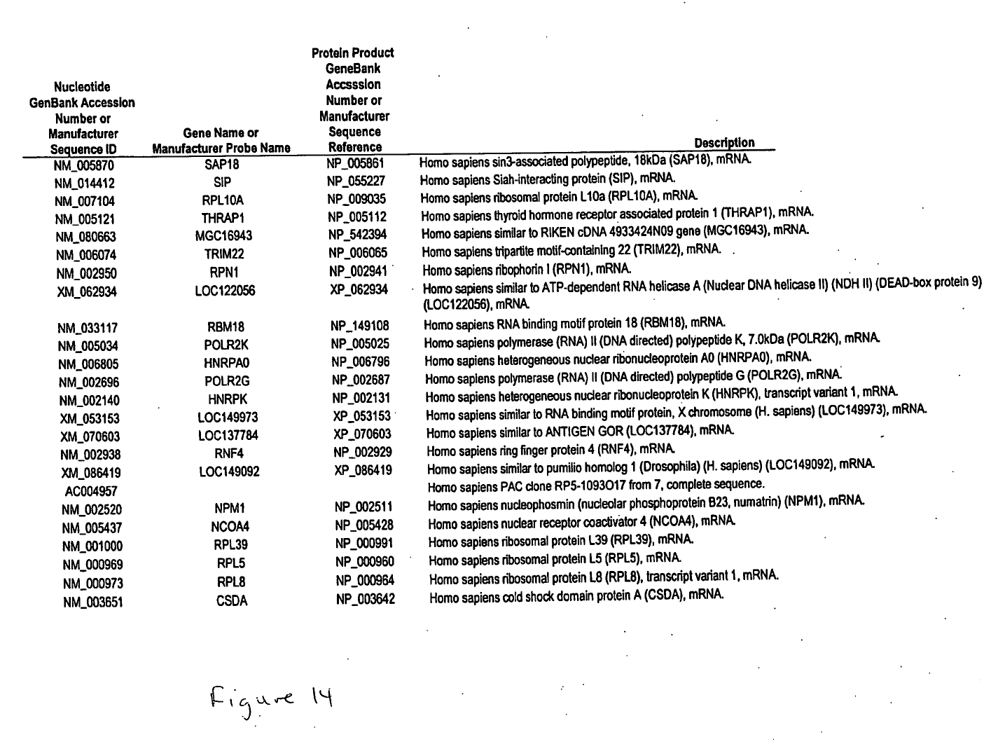

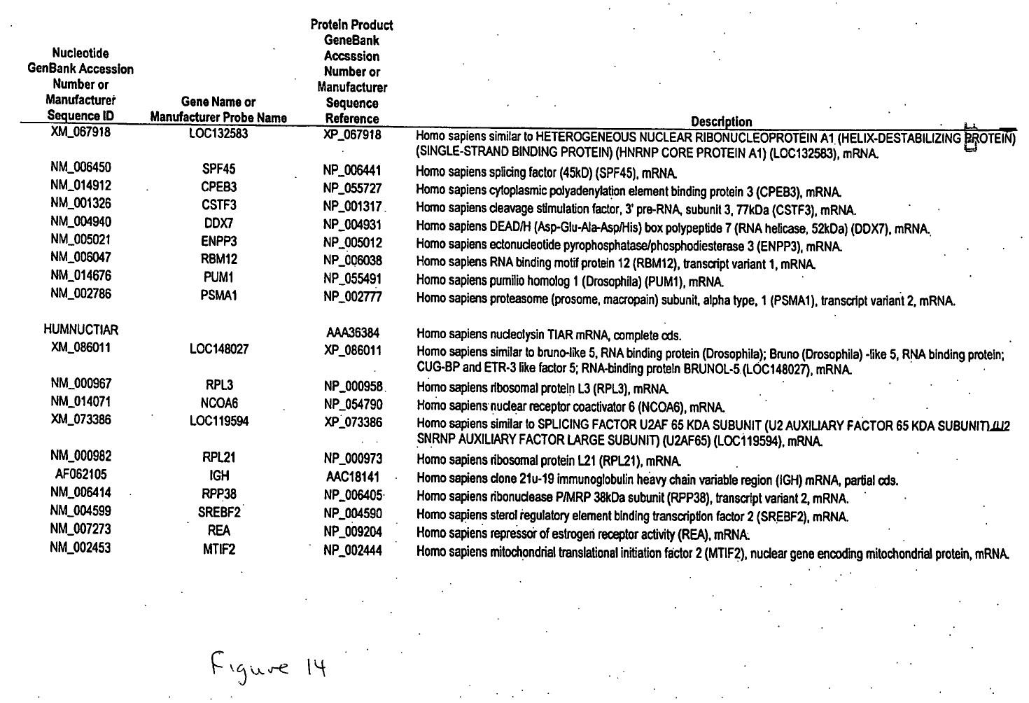

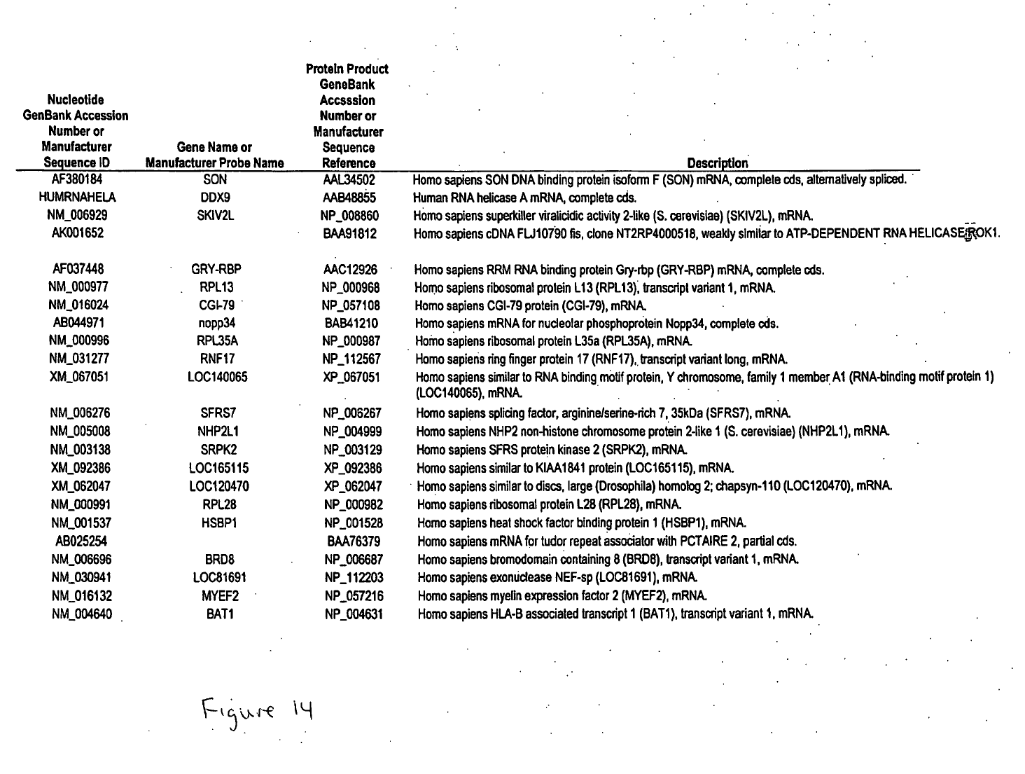

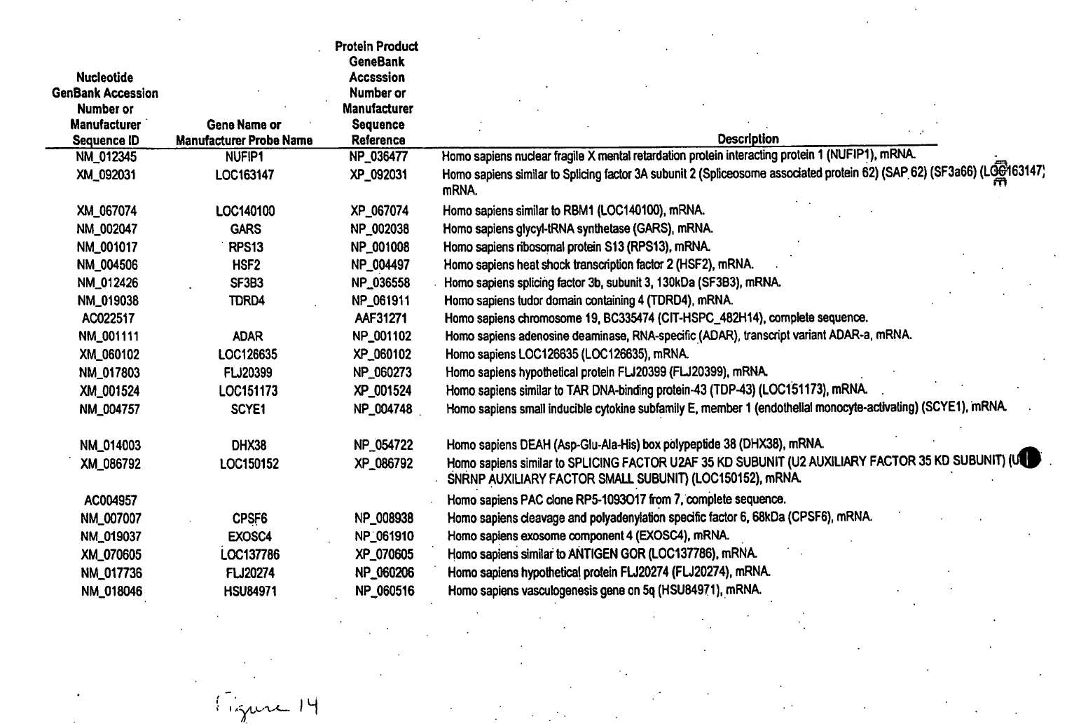

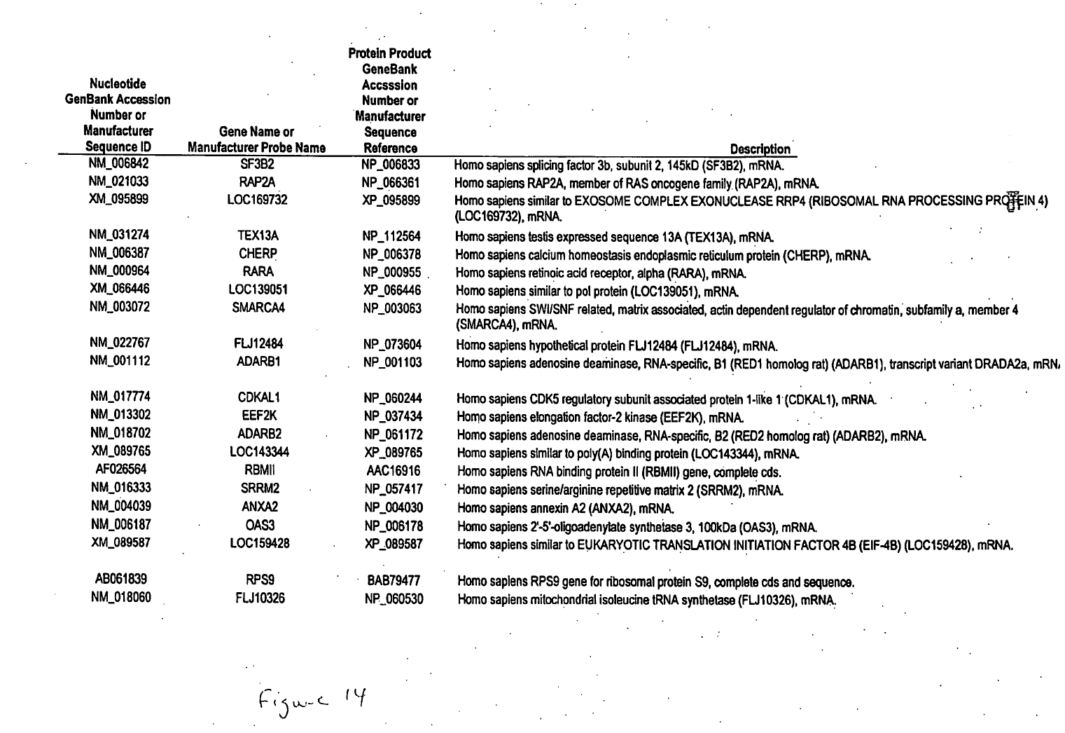

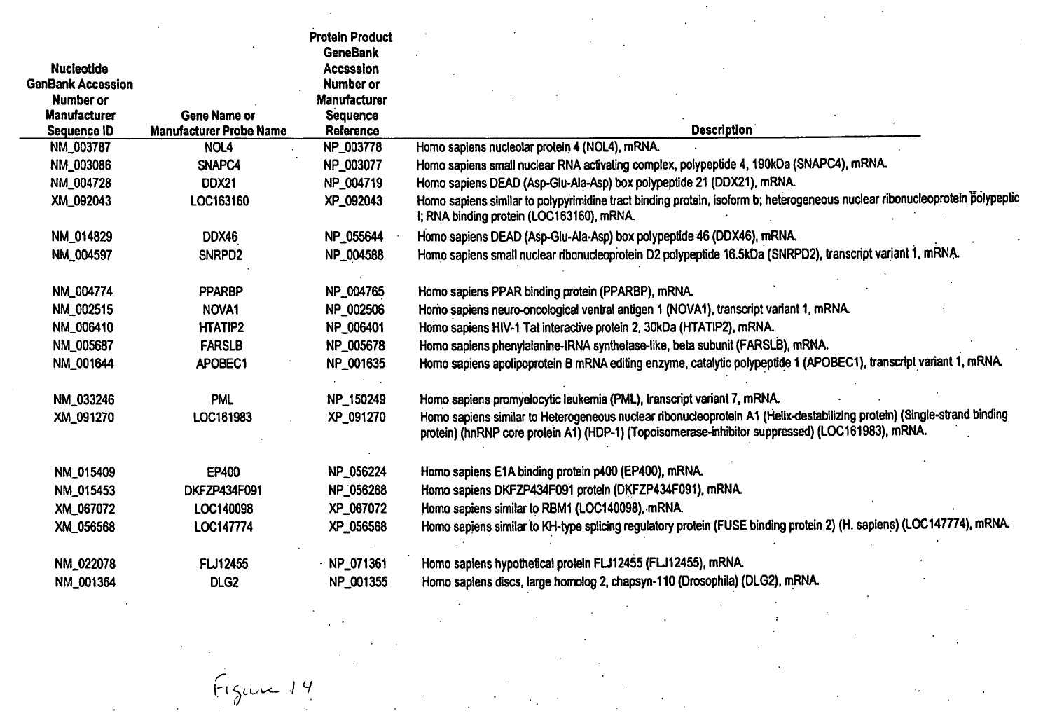

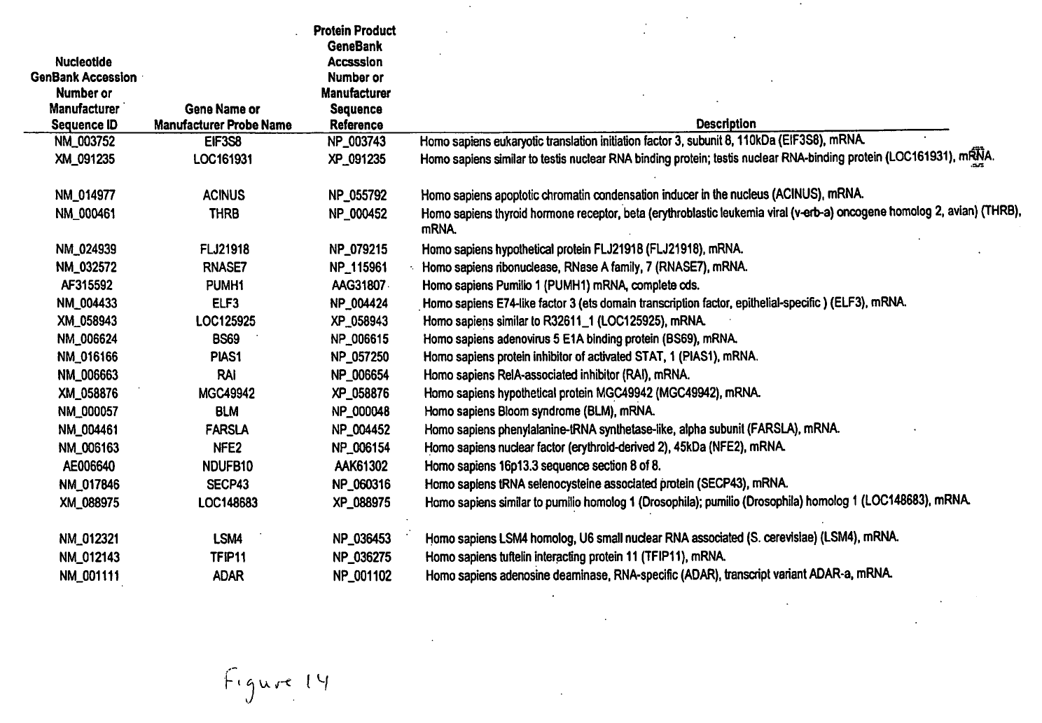

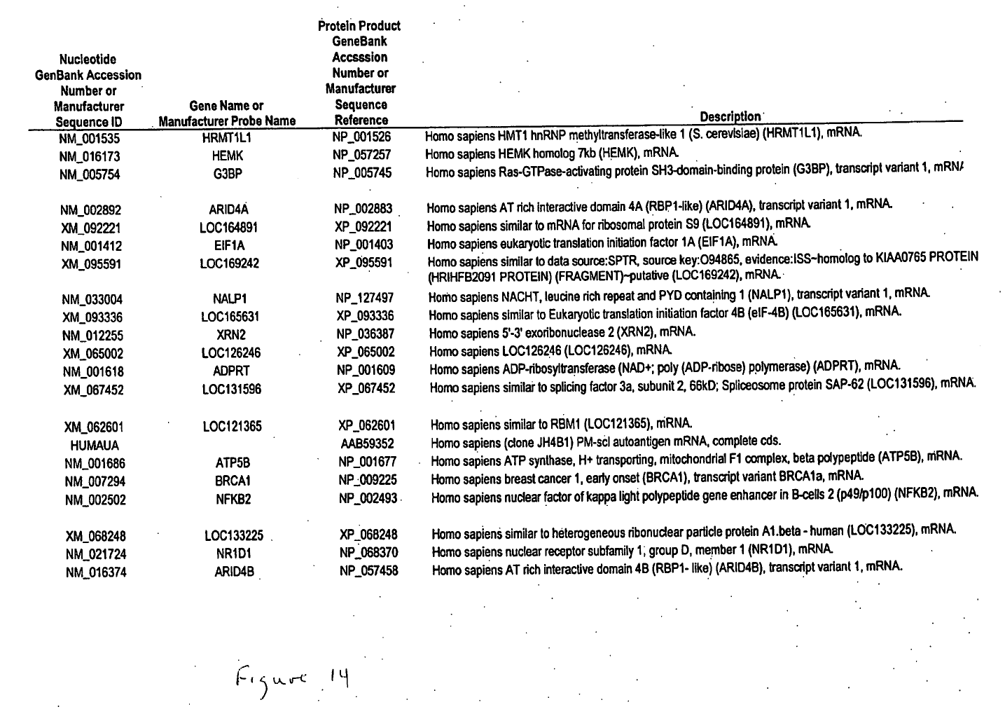

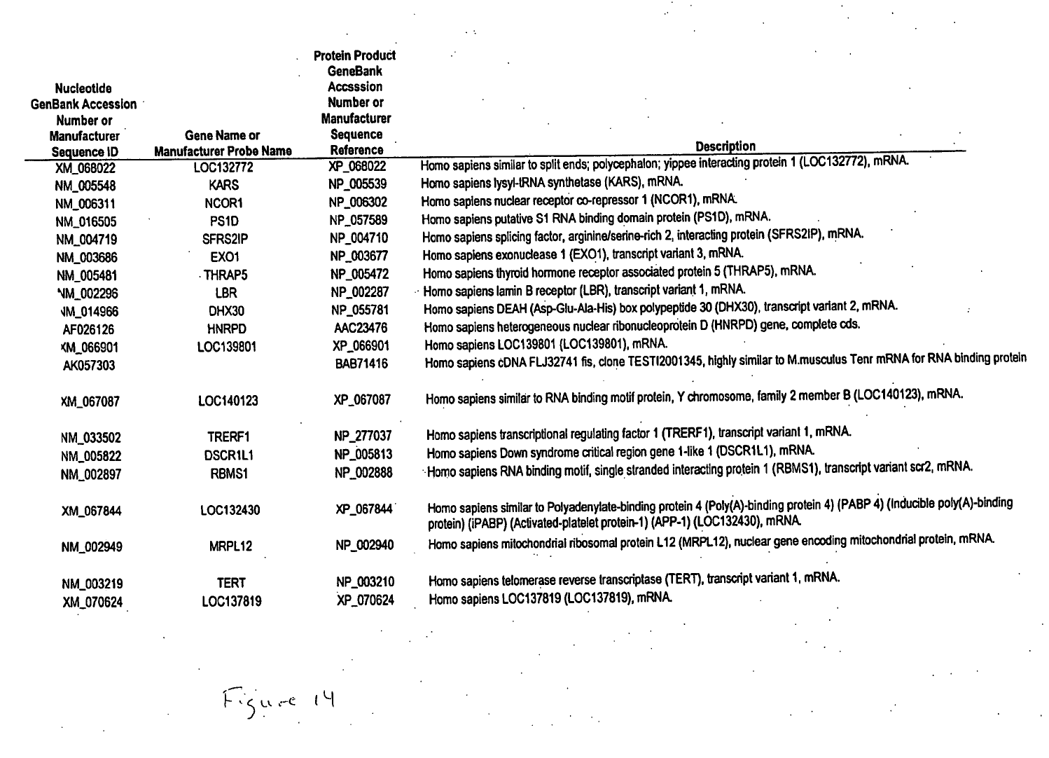

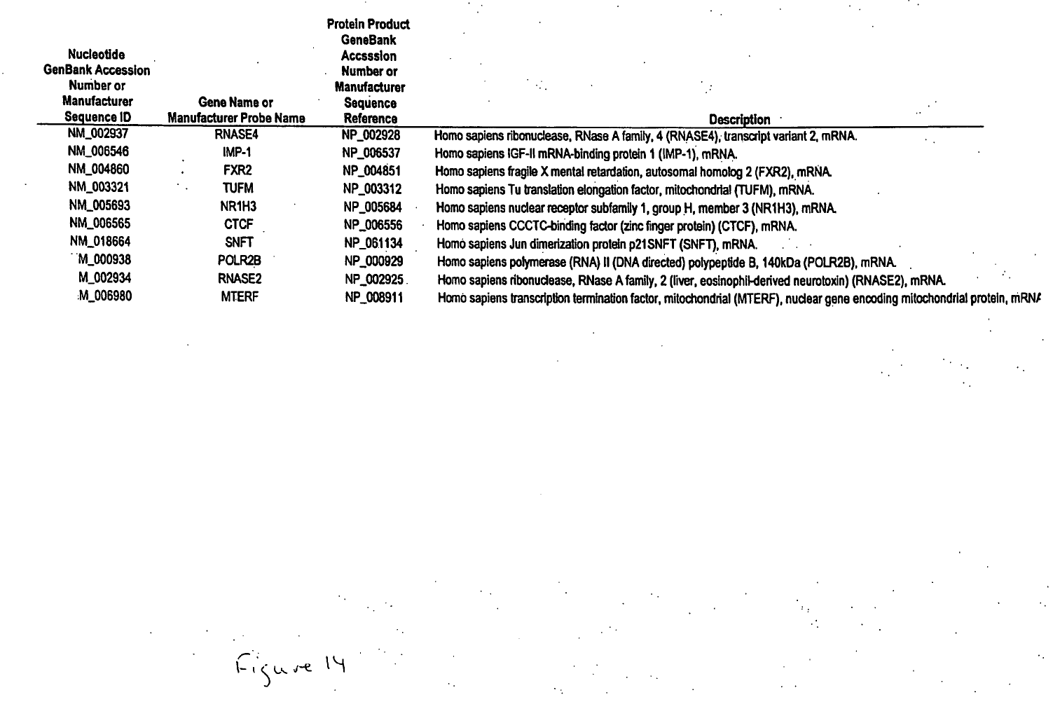

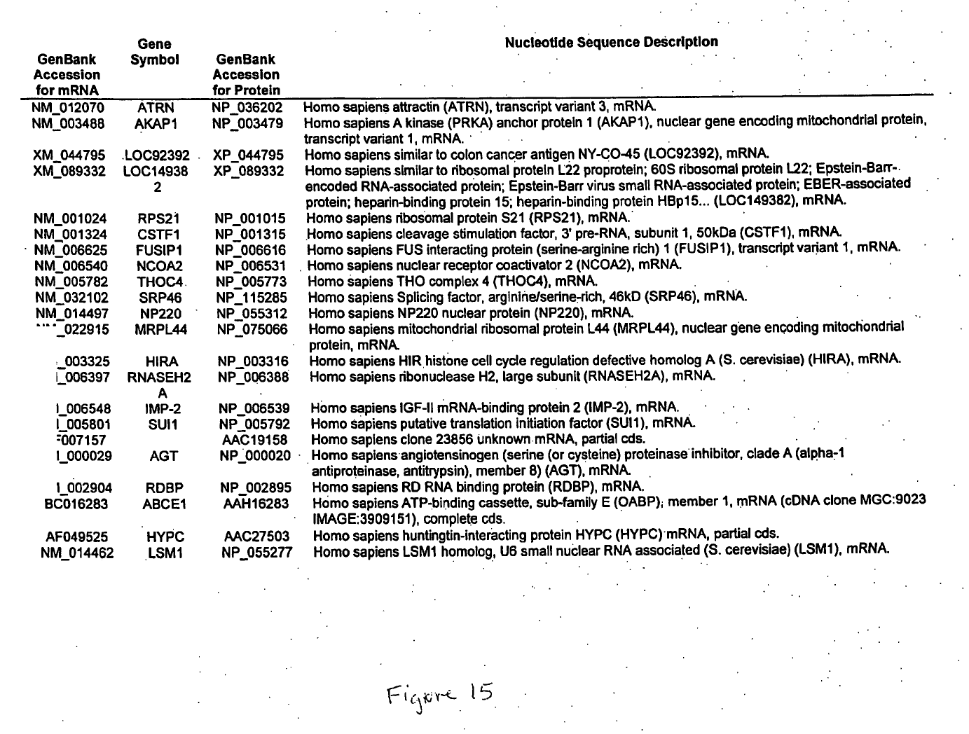

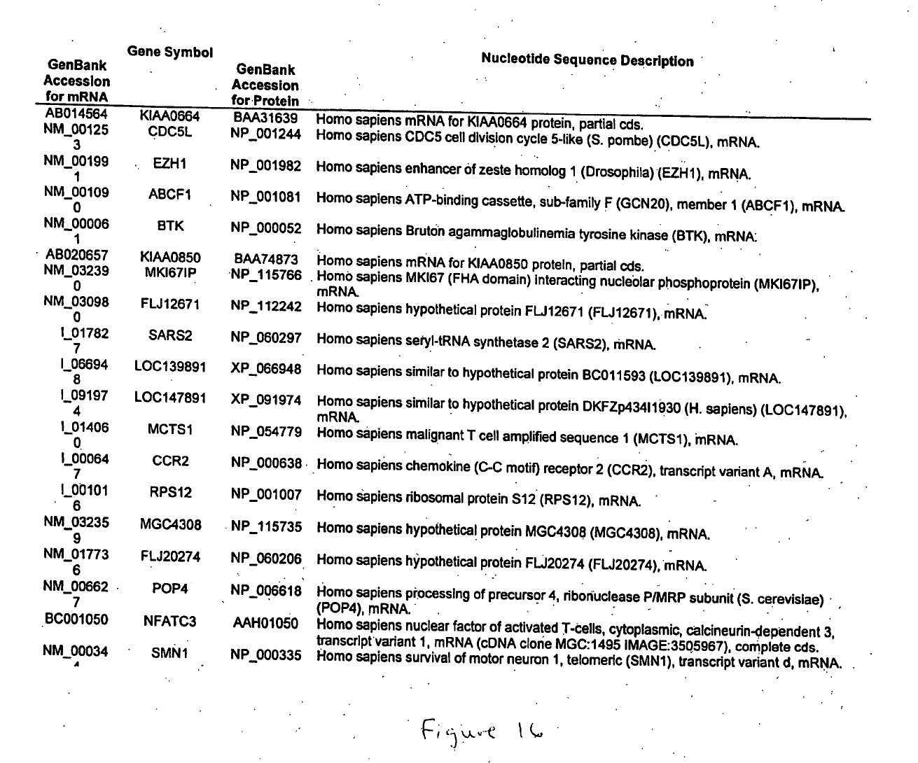

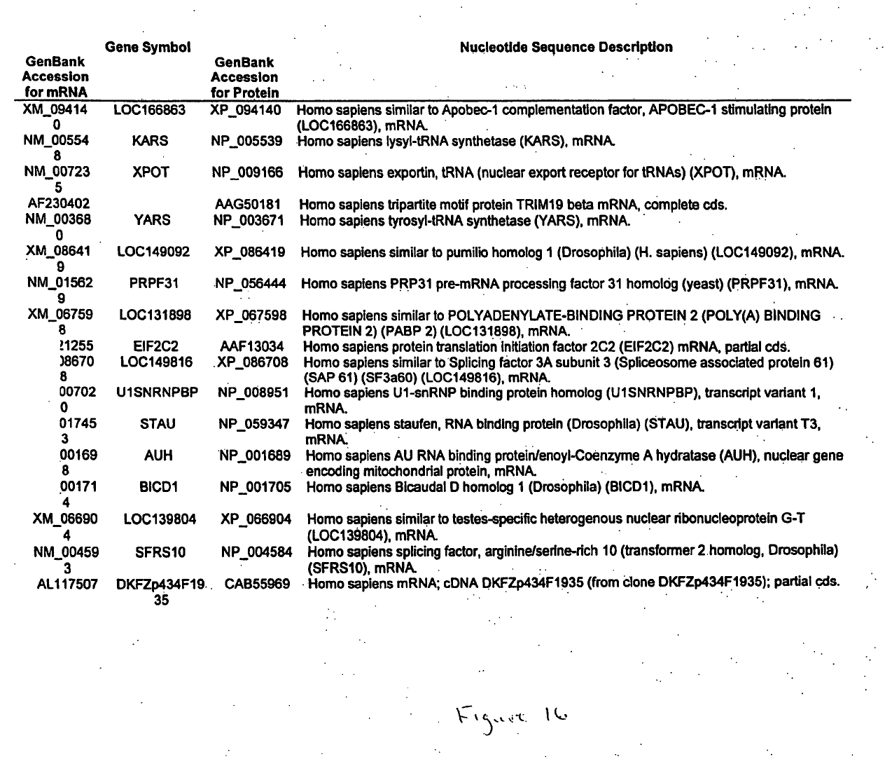

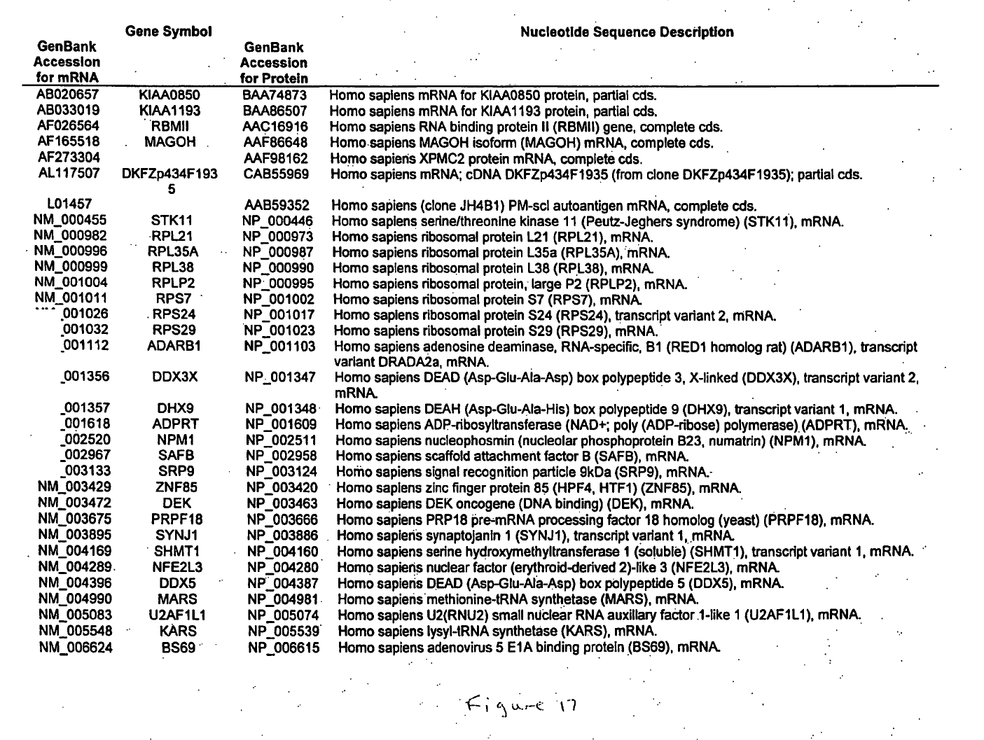

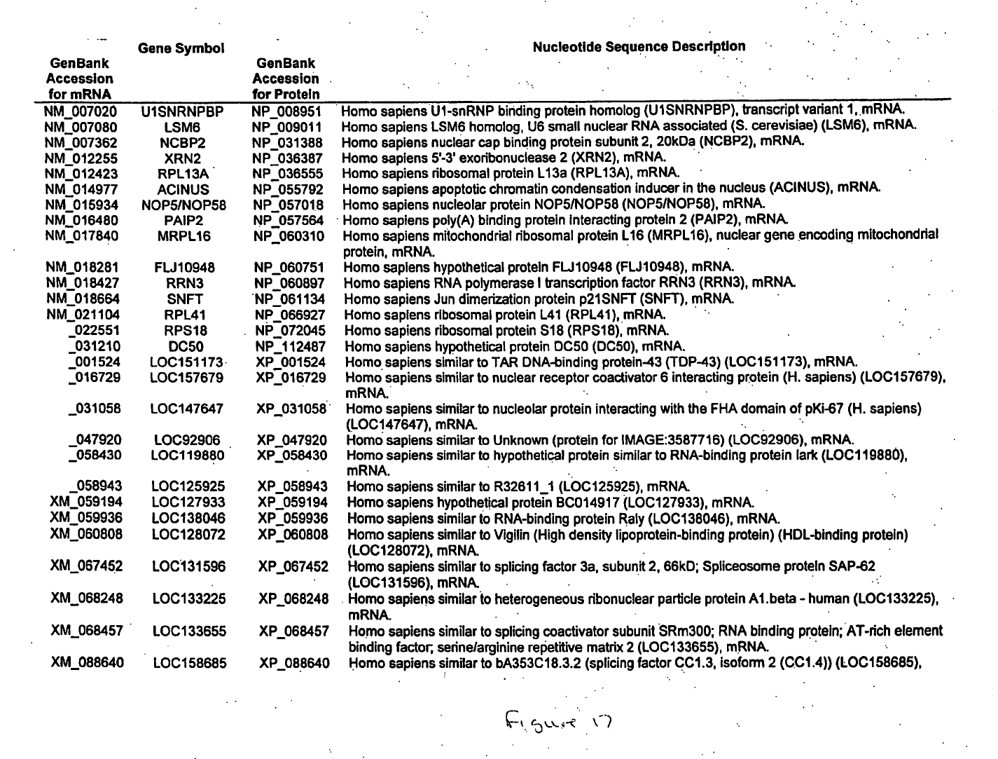

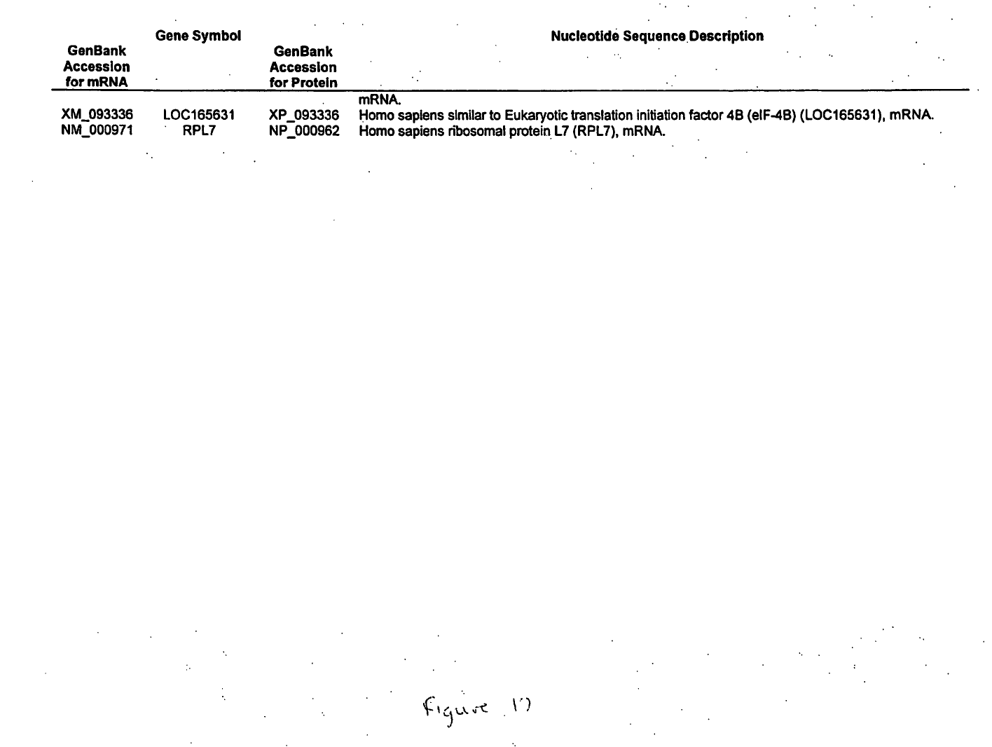

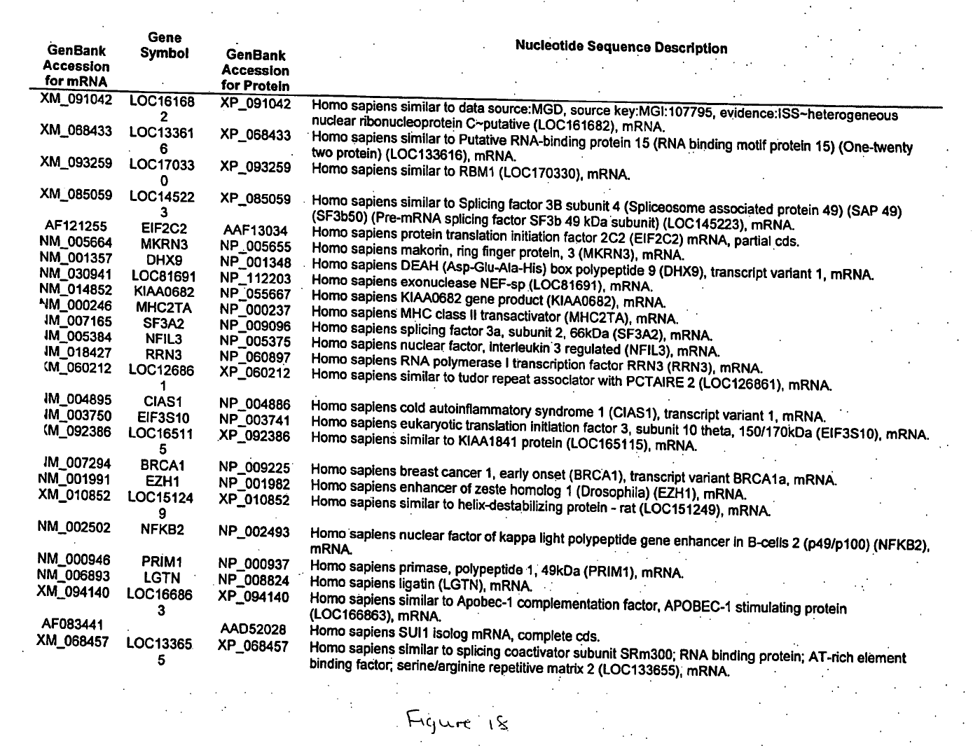

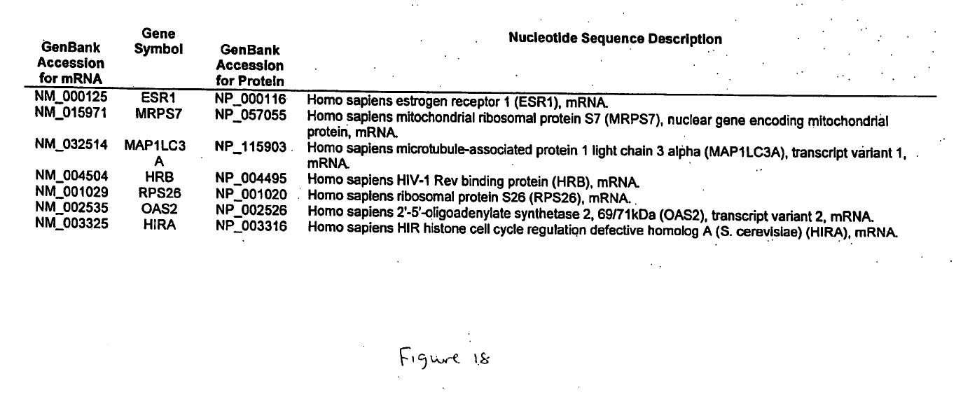

FIG. 1 illustrates an example of an in vitro RIBOTRAP™ method in which a biotinylated mRNA attached to a streptavidin-agarose support is used to identify and isolate an RNA binding protein present in a cell extract, according to standard methods.

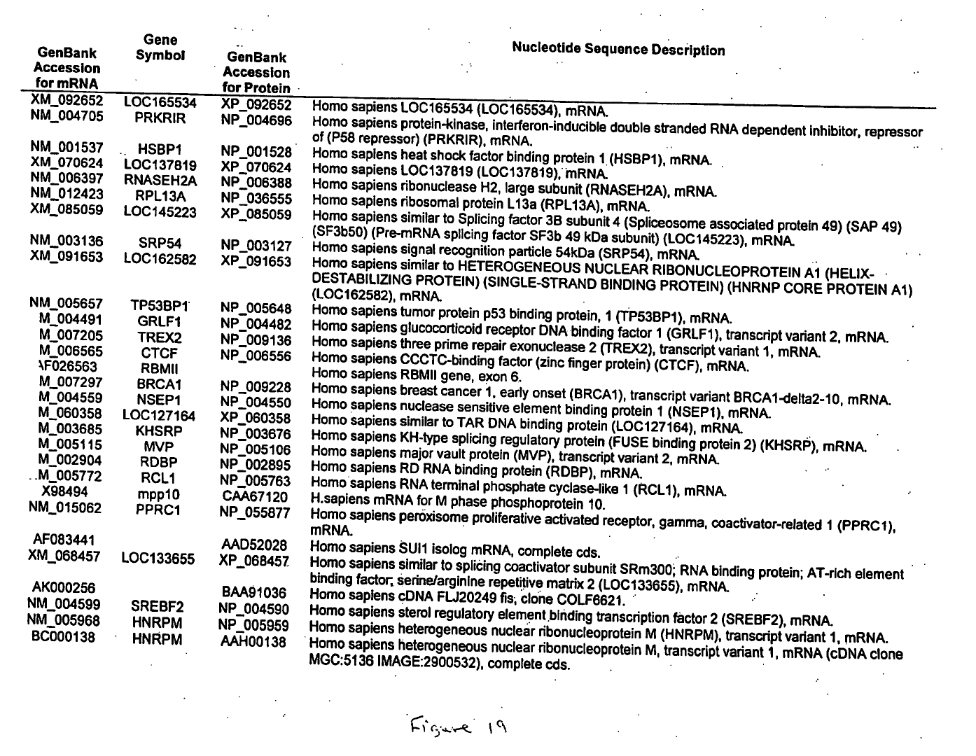

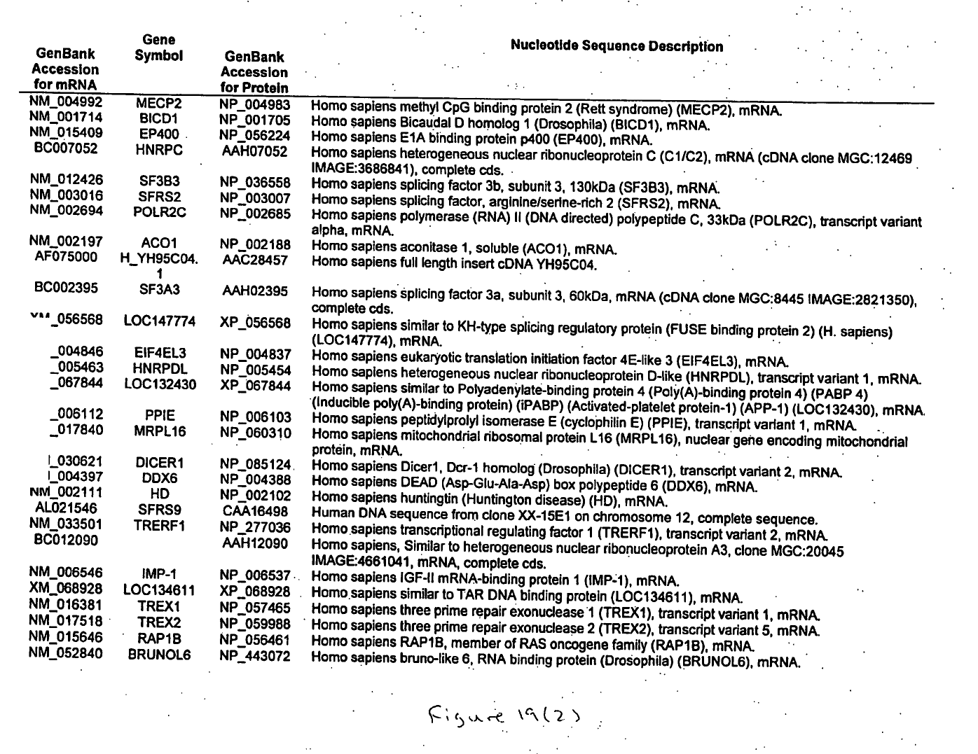

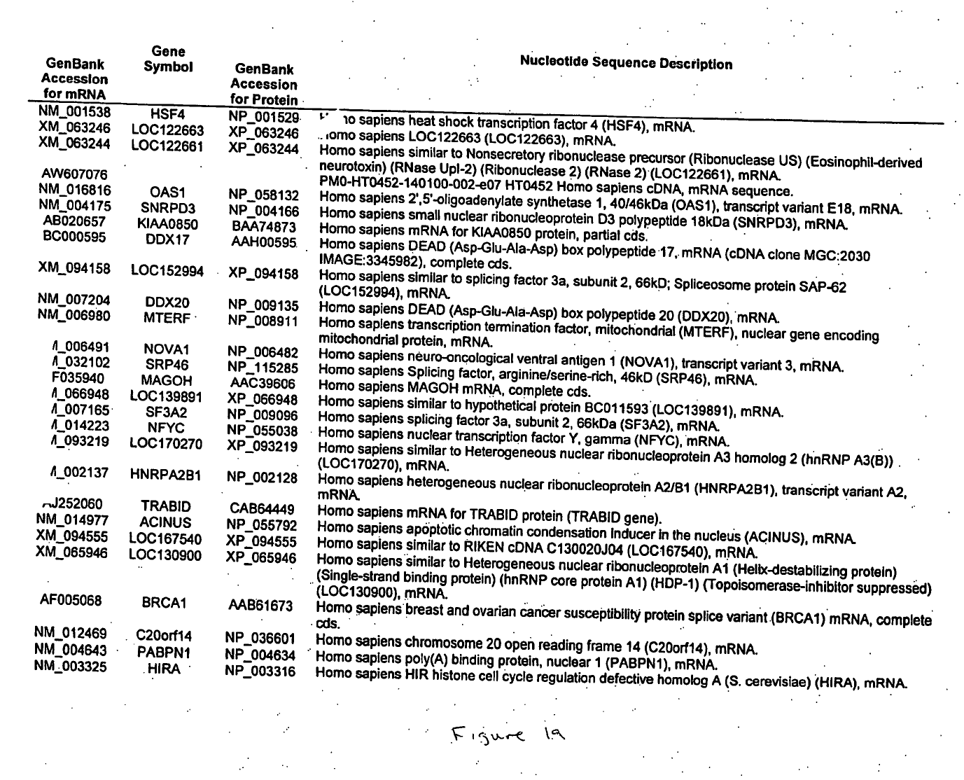

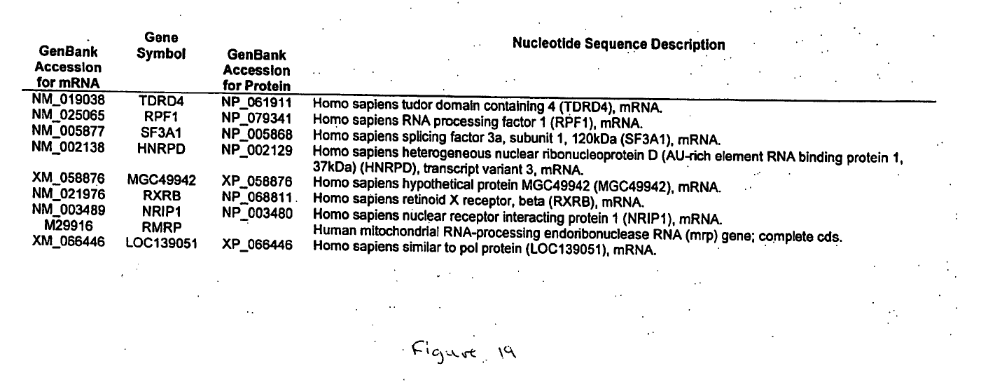

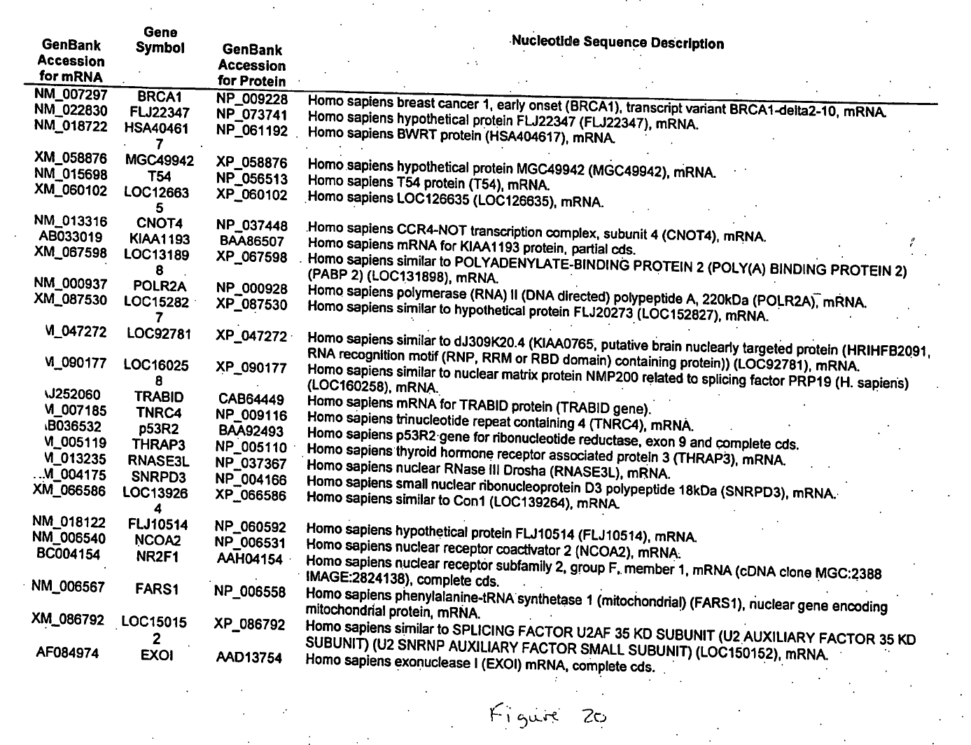

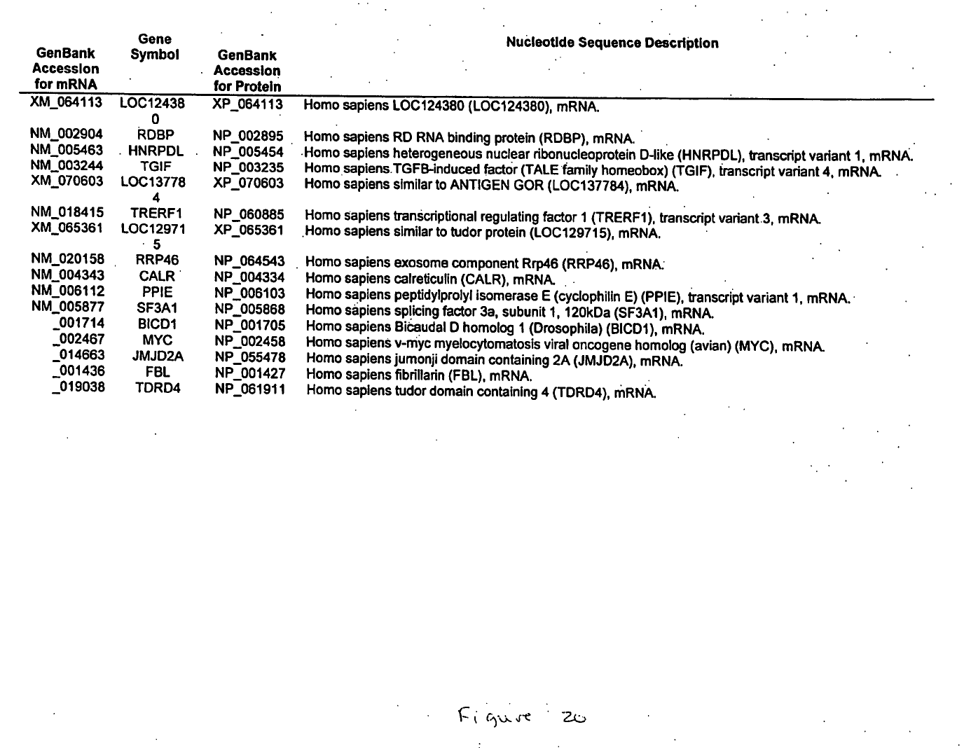

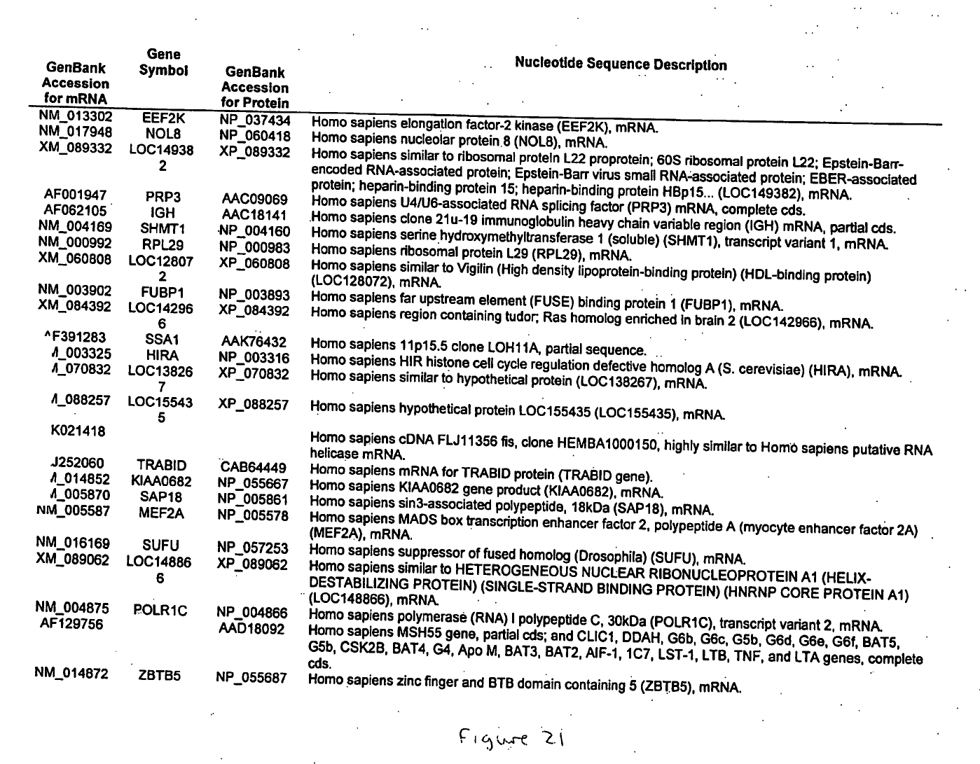

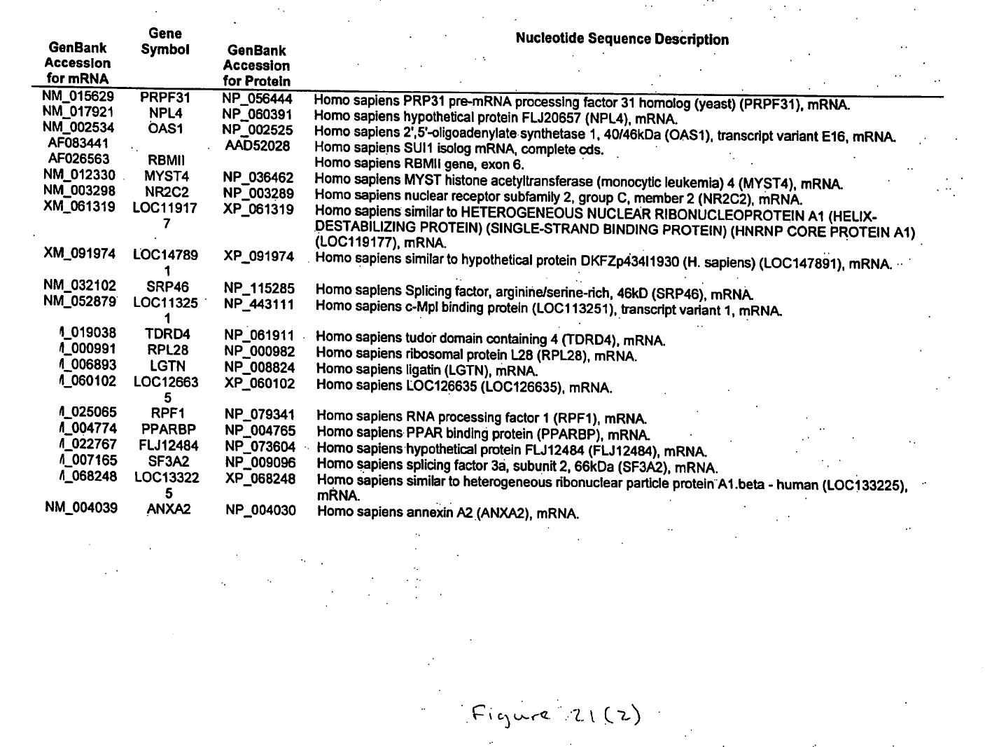

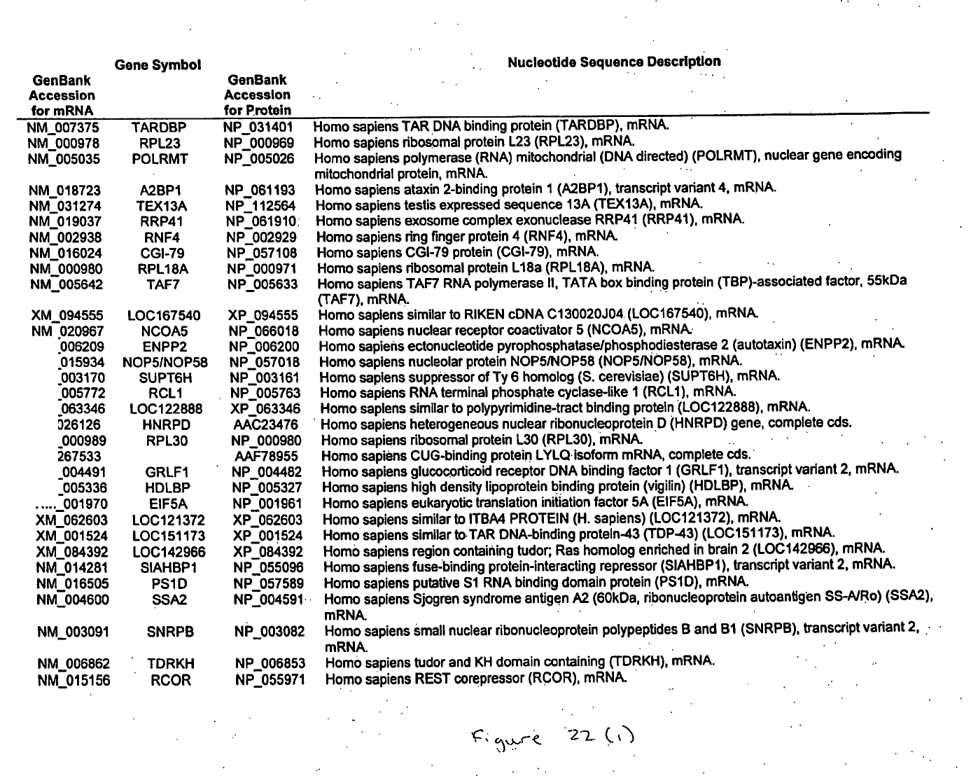

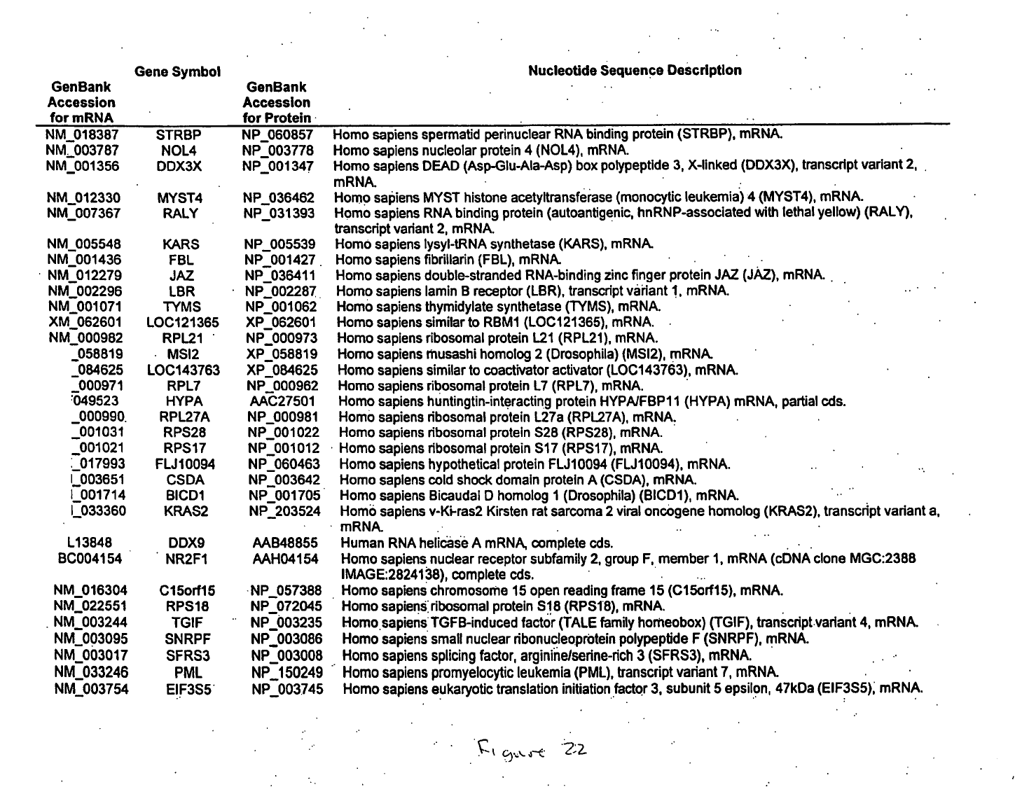

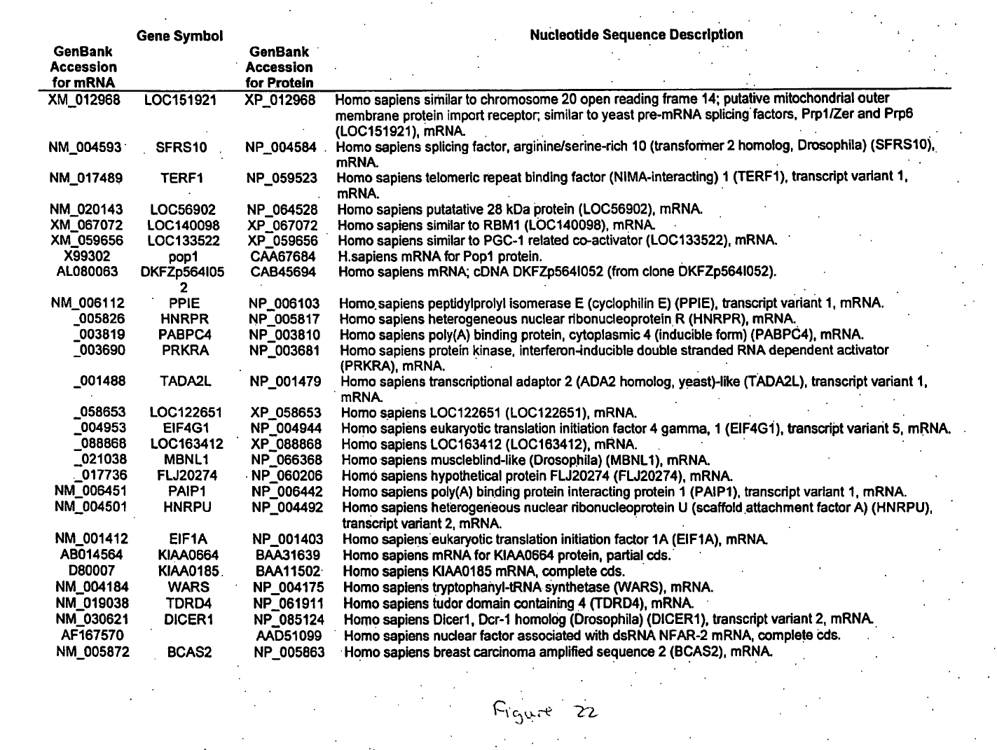

FIG. 2 illustrates one embodiment of the invention, in which an mRNA or portion of a mRNA of interest, “RNA Y”, is used as “bait” to trap a new RNA binding protein (hexagon). Preferably, RNA Y is first converted to a cDNA using standard molecular biology techniques and is subsequently ligated at the 3′ or 5′ end to a DNA tag (dotted lines) that encodes a sequence [symbol] that will bind a ligand (Protein “X”). The resulting fusion RNA is expressed in cells, where endogenous RNA binding proteins can bind and interact with RNA Y. The cells are then lysed and cell-free extracts are prepared and contacted with Protein X, which has been immobilized o [symbol] a solid support. After incubation, Protein X and the attached RNA fusion molecule and its associated RNA binding proteins are washed to remove residual cellular material. After washing, the newly isolated RNA binding proteins are removed from the RNA-protein complex and identified by protein microsequencing or Western blotting. Useful ligands include mRNP complex-specific antibodies or proteins (e.g., obtained from a subject with an autoimmune disorder or cancer). The RNA binding protein is further tested for its ability to regulate the translation of the protein encoded by RNAY, and is tested for validation as a drug target.

In an embodiment, an RNA binding protein is isolated by RIBOTRAP™ from a natural biological sample such as an islet, a pancreatic beta cell, an adipocyte, a preadipocyte, a skeletal muscle cell, a cardiac muscle cell, a hepatocyte, or a population of cells. The population of cells may contain a single cell type. Alternatively, the population of cells may contain a mixture of different cell types from either primary or secondary cultures or from a complex tissue, such as an islet or tumor.

In one embodiment, the RNA binding protein is isolated from a cell sample in which the expression of a component of an mRNP complex, or precursor thereof, has been altered, e.g., induced, inhibited, or over-expressed, e.g., by introduction into the sample or other genetic alteration or after treating the cell or tissue with an agent such as glucose, insulin, a beta-adrenergic agonist, insulin-like growth factor-1 (IGF-1), glucagon-like peptide-1 (GLP-1), fatty acid, peroxisome proliferator activated receptor (PPAR) ligands (e.g. thiazolidinediones, fibrates, halogenated fatty acids, and tyrosine derivatives), insulin-like growth factor-2 (IGF-2), an RNAi against an RNA binding protein, an enhancer of RNA binding protein expression, a nucleic acid, a hormone, an antibody, an antibody fragment, an antigen, a cytokine, a growth factor, a pharmacological agent (e.g., chemotherapeutic, carcinogenic), a chemical composition, a protein, a peptide, and/or a small molecule. Where the compound is a nucleic acid, the nucleic acid may be a DNA, RNA, a PNA, an antisense nucleic acid, a ribozyme, an RNAi, an mRNA, an ncRNA, an rRNA, an siRNA, an snRNA, an snoRNA, an stRNA, a tRNA, an aptamer, a decoy nucleic acid, or a competitor nucleic acid, for example. In one embodiment, the compound may alter the expression of an mRNP complex component through competitive binding. A compound may inhibit binding between two or more mRNP complex components, such as between an RNA binding protein and an RNA, between an RNA binding protein and an mRNP complex-associated protein, between an RNA and an mRNP complex-associated protein, or between two RNAs, RBPs, or mRNP complex-associated proteins, for example. In another embodiment, the cell sample is infected with a pathogen, such as a virus, bacteria, prion, fungus, parasite, or yeast, for example, to alter expression of one or more mRNP complex components. Introduction of a nucleic acid encoding one or more mRNP complex components may be achieved by infection, transformation, or other similar methods known in the art. In one embodiment, an expression vector expressing one or more components of an mRNP complex is transfected into a cell. Suitable vectors include, but are not limited to, recombinant vectors such as plasmid vectors or viral vectors. The nucleic acid encoding the component is preferably operatively linked to appropriate promoter and/or enhancer sequences for expression in the cell. In an embodiment of the invention, a specific cell type is engineered to contain a cell type-specific or inducible gene promoter that drives expression of an RNA binding protein.

Alternatively, a knock-out cell line or knock-out organism may be produced, which either [symbol] does not express a component of an mRNP complex or expresses decreased levels of the component. Preferably, the knock-out cell line or knock-out organism does not express a particular RNA binding protein, mRNA, and/or mRNP complex-associated protein associated with the mRNP complex.

In a preferred embodiment, the nucleic acid encoding the mRNP complex component is tagged in order to facilitate the separation, and/or detection, and/or measurement of the components. Accessible epitopes may be used or, where the epitopes on the components are inaccessible or obscured, epitope tags on ectopically expressed recombinant proteins may be used. Suitable tags include, but are not limited to, biotin, the MS2 protein binding site sequence [symbol] the U1snRNA 70k binding site sequence, the U1snRNA A binding site sequence, the g10 binding site sequence (Novagen, Inc., Madison, Wis.), and FLAG-TAG® (Sigma Chemical, St. Louis, Mo.). For example, a cell is transfected with a vector directing the expression of a tagged [symbol] RNA binding protein and a ligand, such as an antibody or antibody fragment, that is specific for the tag, is used to immunoprecipitate the tagged RNA binding protein with its associated mRNAs from a tissue extract containing the transformed cell.

The expression of one or more mRNP complex components may be altered by contacting [symbol] or treating the cell sample with a known or test compound. The compound may be, but is not limited to, a protein, a nucleic acid, a peptide, an antibody, an antibody fragment, a small molecule, an enzyme, or agents such as glucose, insulin, a beta-adrenergic agonist, insulin-like growth factor-1 (IGF-1), glucagon-like peptide-1 (GLP-1), fatty acid, peroxisome proliferator activated receptor (PPAR) ligands (e.g. thiazolidinediones, fibrates, halogenated fatty acids, and tyrosine derivatives), insulin-like growth factor-2 (IGF-2), RNAi against a RNA binding protein [symbol] an enhancer of RNA binding protein expression, and/or a small molecule (e.g., a putative drug).

RAS™

Once partial sequence of the RNA binding protein is obtained, the corresponding gene may be identified from known databases of cDNA and genomic sequences or isolated from a cDNA or genomic library and sequenced according to art known methods. Preferably, the gene is isolated, the protein is expressed.

Once an RNA binding protein of interest is identified, an antibody is generated against the recombinant RNA binding protein using known techniques. The antibodies are then used to recover and confirm the identity of the endogenous RNA binding protein. Subsequently, the antibody can be used for the Ribonomic Analysis System (RAS™) whereby the mRNP complex [symbol] containing the RNA binding protein is isolated and the subset of cellular RNAs that are associated with the mRNP complex and RNA binding protein are identified by microarray analysis, which is illustrated in FIG. 3 and described in more detail below.

While any method for the isolation of an mRNP complex or its components may be used [symbol] in the present invention, the methods described herein or in U.S. Pat. No. 6,635,422 or disclosed in co-pending U.S. application Ser. Nos. 10/238,306 and 10/309,788 are preferred. For example, in vivo methods for isolating an mRNP complex involve contacting a biological sample [symbol] that includes at least one mRNP complex with a ligand that specifically binds a component of the [symbol] mRNP complex, such as an RNA binding protein. For example, the ligand may be an antibody, a nucleic acid, or any other compound or molecule that specifically binds the component of the complex.

In another embodiment, the mRNP complex is separated by binding the ligand (now bound to the mRNP complex) to a binding molecule that specifically binds the ligand. The binding molecule may bind the ligand directly (e.g., a binding partner specific for the ligand), or [symbol] may bind the ligand indirectly (e.g., a binding partner specific for a tag on the ligand). Suitable binding molecules include, but are not limited to, protein A, protein G, and streptavidin. Binding [symbol] molecules may also be obtained by using the serum of a subject suffering from a disorder such as [symbol] an autoimmune disorder or cancer. In an embodiment, the ligand is an antibody that binds a component of the mRNP complex via its Fab region and a binding molecule binds the Fc region of the antibody.

In another embodiment, the binding molecule is attached to a solid support such as a bead, well, pin, plate, or column. Accordingly, the mRNP complex is attached to the support via [symbol] the ligand and binding molecule. The mRNP complex may then be collected by removing it from the support (e.g., by washing or eluting it from the support using suitable solvents and conditions that are known to a skilled artisan).

In certain embodiments, the mRNP complex is stabilized by cross-linking prior to binding the ligand thereto. Generally, cross-linking involves covalent binding (e.g., covalently binding the components of the mRNP complex together). Cross-linking may be carried out by physical means (e.g., by heat or ultraviolet radiation), or chemical means (e.g., by contacting the [symbol] complex with formaldehyde, paraformaldehyde, or other known cross-linking agents), methods of which are known to those skilled in the art. In another embodiment, the ligand is cross-linked [symbol] to the mRNP complex after binding to the mRNP complex. In additional embodiments, the binding molecule is cross-linked to the ligand after binding to the ligand. In yet another embodiment, the binding molecule is cross-linked to the support.

The methods of the invention allow for the isolation and characterization of a plurality of [symbol] mRNP complexes simultaneously (e.g., “en masse”). For example, a biological sample is contacted with a plurality of ligands each specific for different mRNP complexes. A plurality of [symbol] mRNP complexes from the sample bind the appropriate specific ligands. The plurality of mRNP [symbol] complexes are then separated using appropriate binding molecules, thereby isolating the plurality [symbol] of mRNP complexes. The mRNP complexes and the mRNAs contained within the mRNP complexes are then characterized and/or identified by methods described herein and known in the art. Alternatively, the methods of the invention are carried out on a sample numerous times and the mRNP complexes are characterized and identified in a sequential fashion, with each iteration utilizing a different ligand.

Following isolation of an mRNP complex, the level of expression of at least one mRNA associated with the mRNP complex is determined. The collection of mRNAs, together with the [symbol] RNA binding proteins, and mRNP complex-associated proteins on a particular mRNP complex provides a ribonomic profile, that is indicative of the gene expression of a subset of functionally [symbol] related gene products. It will be appreciated that ribonomic profiles differ from cell to cell as described previously. Thus, a ribonomic profile for one cell type can be used as an identifier for [symbol] that cell type and can be compared with ribonomic profiles of other cells.

FIG. 4 illustrates an embodiment of the invention in which the RAS™ technology is used in conjunction with a RIBOTRAP™ method to identify functionally and/or structurally related mRNAs associated with an mRNP complex. FIG. 4 shows a comparison of the data obtained using traditional analysis of total RNA compared to the data obtained using RIBOTRAP™ to first isolate a particular RNA binding protein is followed by the use of RAS™ [symbol] to identify associated mRNAs. The use of RIBOTRAP™ and RAS™ provides a more sensitive [symbol] assay that is enriched for the subset of RNAs associated with a particular RNA binding protein and which are likely functionally related. By comparison, microarray analysis of total RNA does [symbol] not provide the same level of sensitivity and functionality and provides a more complex data se

[symbol]

Amplification of the mRNA isolated according to the methods of the invention and/or the [symbol] cDNA obtained from the mRNA is not necessary or required by the present invention. However [symbol] the skilled artisan may choose to amplify the nucleic acid that is identified according to any of the numerous nucleic acid amplification methods that are well-known in the art (e.g., polymerase [symbol] chain reaction (PCR), reverse transcriptase polymerase chain reaction (RT-PCR), quantitative real time polymerase chain reaction (QRT-PCR), rolling circle amplification (RCA), or strand displacement analysis (SDA)).

One goal of the RAS™ assay is to identify mRNAs that encode proteins that have functional relationships. Among the related functions that are expected are a) involvement of encoded proteins in a common metabolic pathway, b) encoded proteins that are temporally co-regulated, c) encoded proteins that are similarly localized in or on the cell, d) encoded proteins that play a role in forming or regulating a biological machine (e.g., a ribosome). The identification of complex traits and phenotypes that result from the expression of a set of functionally-related proteins would include such processes as cognition, cell-specific activation, inflammation, or differentiation. While proteins known to be involved in these complex processes are known from other studies, the majority of the functions remain largely unknown. One of the values of the invention is for discovering a larger set of proteins involved in these processes that could serve as alternative drug targets or surrogate markers.

In addition, the subpopulation of mRNAs that are present in an mRNP complex can be identified and examined for the presence of common sequence elements, such as 5′ or 3′ untranslated regions, or common functional features. RAS™ can then be used to identify the unique subsets of RNAs associated with those RNA binding proteins. Computational analysis of [symbol] the primary sequence for identifying Untranslated Sequence Elements for Regulation Codes (USER codes) may be used alone or in combination with secondary structure analysis. In addition, the subpopulation of mRNAs can be examined for functional relationships. For example, each mRNA can be categorized by gene annotation and by known functions in functional genomics databases (e.g., Locus Link (NCBI, Bethesda, Md.), GO Database (Gene Ontology™ Consortium), Proteome BioKnowledge® Library (Incyte Genomics, Inc., Palo Alto Calif.)). For example, if the RNA binding protein or mRNP complex is involved in immune regulation, the other mRNAs found in the same mRNP complex can be analyzed for their role in [symbol] immune regulation. However, the mRNA could be bound indirectly through a different RNA binding protein or RNA in the mRNP complex (e.g., is assessed for the presence of the USER code element in its UTR that recognizes the RNA binding protein or other known binding sites for RNA binding proteins).

An exemplary technique for isolating functional clusters of mRNAs is in vivo RAS™, whereby the unique repertoire of mRNAs (defined herein as a “functional cluster”) that is associated with a particular RNA binding protein in vivo is identified. Alternatively, in vitro RAS™ may be used, wherein the RNA binding proteins and mRNAs are associated in vitro and analyzed. The in vitro technique is useful if, for example, the RIBOTRAP™ technique for isolating endogenous RNA:protein complexes is not feasible, for example due to ineffective affinity reagents for immunoprecipitation of the intact endogenous complex.

In Vitro RAS™

Example 5 provides examples of methods for performing in vitro RAS™. Briefly, an RNA binding protein is cloned by polymerase chain reaction (PCR) and the sequence verified and expressed in E. coli as a glutathione S transferase (GST) fusion protein. Following purification, the GST-RNA binding protein was attached to glutathione Sepharose beads and exposed to mRNA preparations to assess its ability to selectively retain discreet mRNA pools. Messenger RNA retained by an individual GST-RNA binding protein was profiled by combined microarray and QRT-PCR analyses, according to standard methods. Messenger RNA untranslated region (UTR) sequences are aligned to search for obvious consensus elements in the retained mRNA pools, and a small number (e.g., 5-10 UTRs) are initially evaluated to confirm direct binding by biotinylated oligonucleotide-affinity chromatography (as described for RIBOTRAP™).

In general, two types of mRNA preparations are used, purified cytoplasmic RNA and cleared cytoplasmic lysates. Purified cytoplasmic RNA is used to directly identify mRNAs that encode cis binding elements for the RNA binding protein. Cellular lysates containing both RNA and protein may have improved specificity of the RNA binding protein:RNA interaction, for example, due to the presence of auxiliary factors that modulate binding.

For additional glucose and/or lipid-regulated RNA binding proteins, comparisons are made between mRNA pools retained using purified RNA or cytoplasmic lysates (as described for RAS™) prepared from cells or tissue treated with an agent such as glucose, insulin, a beta-adrenergic agonist, insulin-like growth factor-1 (IGF-1), glucagon-like peptide-1 (GLP-1), fatty acid, peroxisome proliferator activated receptor (PPAR) ligands (e.g. thiazolidinediones, fibrates, halogenated fatty acids, and tyrosine derivatives), insulin-like growth factor-2 (IGF-2), RNAi against a RNA binding protein, an enhancer of RNA binding protein expression, and/or a small molecule (i.e., a putative drug).

Example 6 describes an example of in vitro RAS™. In short, human PTB was cloned into a glutathione S transferase vector and recombinant protein (GST-PTB) was purified as known to those skilled in the art. GST-PTB was immobilized onto glutathione Sepharose beads and incubated with cleared cytoplasmic lysates or purified RNA prepared from pancreatic beta cells. The matrix is washed thoroughly with binding buffer and RNAs bound to GST-PTB were purified. As a control, the same RNA preparations were incubated with a glutathione bound matrix containing GST protein alone or another GST-RNA binding protein. The purified RNA from each column was identified by microarray analysis or QRT-PCR.

In Vivo RAS™

In another embodiment of the invention, endogenous mRNP complexes from cells or tissue are profiled by immunoprecipitation of endogenous mRNP complexes from cell lysates and characterization of mRNA content. A binding partner (e.g., an antibody) to an individual RNA binding protein or other mRNP complex component is used to isolate the mRNP complex and identify and characterize the associated mRNAs, e.g., during any given disease state or under certain experimental conditions. In contrast to the tagged RNA binding protein approach described for in vitro RAS™ isolation of endogenous RNA binding protein complexes does not require transfection and selection of cell lines expressing tagged RNA binding proteins prior to analysis. However, in vivo RAS™ analysis requires antibodies specific for individual RNA binding proteins or other mRNP complex component that can immunoprecipitate intact endogenous mRNP complexes. Polyclonal anti-peptide and\or full-length protein antibodies, monoclonal antibodies, or recombinant antibody libraries specific for a mRNP complex component such as an RNA binding protein may be used. For example, a commercial antibody for the RNA binding protein PTB (Zymed, South San Francisco, Calif.) was used to effectively immunoprecipitate PTB-containing mRNP complexes from INS-1 cells.

Antibodies and fragments thereof that bind to mRNP complexes are generated using methods that are well known in the art. Such antibodies may include, but are not limited to, polyclonal, monoclonal, chimeric, single chain, Fab fragments, and fragments produced by a Fab [symbol] expression library. Antibodies and fragments thereof may also be generated using antibody phage expression display techniques, which are known in the art.

For the production of antibodies, various hosts including, but not limited to, goats, pigs, rabbits, rats, chickens, mice, and humans are immunized by injection with the mRNP complex o [symbol] any fragment or component thereof that has immunogenic properties. Depending on the host species, an adjuvant is used to increase the immunological response. Such adjuvants include, but [symbol] are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, Bacilli Calmette-Guerin and Corynebacterium parvum are preferable.

Monoclonal antibodies to the components of the mRNP complex are prepared using any technique that provides for the production of antibody molecules by a cultured cell line. These include, but are not limited to, the hybridoma technique, the human B-cell hybridoma technique, and the EBV-hybridoma technique. Generally, an animal is immunized with the mRNP complex [symbol] or immunogenic fragment(s) or conjugate(s) thereof. Lymphoid cells (e.g., splenic lymphocytes) [symbol] are then obtained from the immunized animal and fused with immortalized cells (e.g., myeloma or heteromyeloma) to produce hybrid cells. The hybrid cells are screened to identify those that produce the desired antibody.

Antibodies may also be produced by inducing in vivo production in the lymphocyte population or by screening immunoglobulin libraries or panels of highly specific binding reagents as is known in the art.

Antibody fragments that contain specific binding sites for mRNP complexes may also be generated. For example, such fragments include, but are not limited to, the F(ab′)₂ fragments that [symbol] can be produced by pepsin digestion of the antibody molecule and the Fab fragments that can be generated by reducing the disulfide bridges of the F(ab′)₂ fragments. Alternatively, Fab expression libraries are constructed to allow rapid and easy identification of monoclonal Fab fragments with the desired specificity.

Various immunoassays are used to identify antibodies having the desired specificity for the mRNP complex. Numerous protocols for competitive binding or immunoradiometric assays using either polyclonal or monoclonal antibodies with established specificities are well known in the art. Such immunoassays typically involve the measurement of complex formation between the component of the mRNP complex and its specific antibody. An immunoassay utilizing monoclonal antibodies reactive to two non-interfering epitopes is preferred, but a competitive binding assay may also be employed.

The antibodies may be conjugated to a support suitable for a diagnostic assay (e.g., a solid support such as beads, plates, slides or wells formed from materials such as latex or polystyrene) in accordance with known techniques. Antibodies may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques. Such devices preferably include at least one reagent specific for detecting the [symbol] binding between an antibody and the RNA binding protein. The reagents may also include ancillary agents such as buffering agents and protein stabilizing agents (e.g., polysaccharides and [symbol] the like). The device may further include, where necessary, agents for reducing background interference in a test, control reagents, apparatus for conducting a test, and the like. The device may be packaged in any suitable manner, typically with all elements in a single container, along with a sheet of printed instructions for carrying out the test.

In an embodiment, full-length RNA binding protein genes are amplified by PCR from appropriate cDNA libraries and cloned into expression vectors (e.g., pGEX or pDEST17 6X-His) for bacterial expression, purification, and antibody production. Antibodies are affinity-purified, characterized, and optimized for immunoprecipitation of the protein and its associated RNA binding proteins or mRNP complex. The ability of the antibody to precipitate RNAs in general is determined by a rapid, high-throughput analysis using a 2100 BioAnalyzer (Agilent, Palo Alto, Calif.). Non-immune controls include previously characterized RNA binding protein antibodies are run in parallel as negative and positive controls, respectively. Specific antisera that are able to immunoprecipitate the RNA binding protein and/or mRNP complex are used for further analysis.

Optionally, more than one peptide antigen may be chosen based on analysis of the protein sequence using software for antigenic determination (Antheprot, Lyon, France; uses Parker and Wellington algorithms), followed by a Blast P search in NCBI to ensure that the designed peptide is not significantly homologous to another protein. Peptides are selected from regions thought to lie outside the RNA binding domain, to enrich for epitopes that are more likely to be exposed in the mRNP complex. In an embodiment, 15-25 amino acid peptides are synthesized according to standard methods and conjugation to Keyhole limpet hemocyanin (KLH), followed by immunization of rabbits for polyclonal antibody production.

RNA binding proteins or mRNP complexes may be immunoprecipitated as follows. In an embodiment, antibodies specific for a particular RNA binding protein/mRNP complex are pre-bound to protein A beads, blocked with bovine serum albumin and washed extensively. After a final wash in lysis buffer, cell extracts are added. Nuclei-free cytosolic extracts are prepared essentially as described from cells (or tissue) that have been exposed to various experimental conditions (e.g., low and high glucose). Incubation times and temperatures are optimized for each anti-RNA binding protein antibody. The complexes are washed under nuclease-free conditions. The antibody-mRNP complex is then disrupted with denaturing buffer RLT (Qiagen, Inc., Valencia, Calif.), containing guanidine thiocyanate, and mRNA purified using Qiagen RNA isolation column chromatography (Qiagen, Inc., Valencia, Calif.). The purified mRNA is then processed for microarray analysis, for example on human or rodent microarrays (depending on the cell or tissue source) comprised of features (e.g., 10,000-40,000 genes) representing up-to-date genomic content (e.g., Affymetrix, Santa Clara, Calif.; Agilent, Palo Alto, Calif. or MWG Biotech, Inc. High Point, N.C.). A gene observed at ‘detectable’ levels that is present in each of the experiments is considered a component of mRNP complex to which it is associated and its relative fold-enrichment above a total RNA microarray analysis is determined. Routinely, genes expressed at a level above local background are considered members of that cluster. The presence of the candidate genes and their relative fold-enrichment over total RNA are verified and more accurately quantified by QRT-PCR using sequence-specific primers.

In an embodiment, the combination of the in vitro and in vivo RAS™ based approaches may be used to map mRNP complex pools and accurately define the RNA content of selected mRNP complexes.

The multicomponent nature of mRNP complexes can interfere with efficient immunoprecipitation due to inaccessibility of reactive polypeptide epitopes. In the absence of appropriate affinity reagents or when endogenous complexes cannot be isolated, mRNAs associated with individual RNA binding proteins in a cell are identified by using RNA binding proteins tagged with one of several generic epitopes such as, for example, Flag, AU1, or T7. The binding epitopes are expressed on the N- or C-terminus of the RNA binding protein and introduced into an appropriate cell line for expression. Pooled cell lines are generated by selection (e.g., in zeocin) and screened for stable expression of the tagged RNA binding proteins Commercially available antibodies (e.g., α-T7, Novagen, Madison, Wis.) are used to immunoprecipitate mRNP complexes from cells, for example, INS-1 cells following mock or glucose treatment. As a positive control, tagged poly A binding protein (PABP1), which is known to bind virtually all polyadenylated mRNAs, is constructed and transfected into INS-1 cells for parallel immunoprecipitation of mRNP complexes. Messenger RNA pools isolated following low and high glucose treatment of the individual INS-1 cell lines (pooled lines) are evaluated by microarray analysis and selective QRT-PCR confirmation. The use of a tagged-RNA binding protein is advantageous in that the functional cluster associated with the tagged-RNA binding protein can be directly compared with that isolated using a commercially available monoclonal antibody to the RNA binding protein. This allows for validation of the endogenous RNA binding protein cluster as well as assessment of the mRNA binding characteristics of the tagged-RNA binding protein.

The mRNA pools were converted into amino allyl cDNAs and labeled with cyanine dyes for use as probes on microarrays. Aminoallyl cDNA (aa-cDNA) was synthesized from RNA preps based on modifications of protocols by DeRisi (www.microarray.org; “Reverse Transcription and aa-UTP Labeling of RNA”) and TIGR (www.tigr.org; Protocol M005), as described in Example 1. Purified aa-cDNA was coupled to cyanine dyes (Amersham Biosciences; Piscataway, N.J.; Catalog # PA23001 (Cy3) or PA25001 (Cy5)), purified, and analyzed as described in Example 1.

For each microarray, material from one Cy3 labeling and one Cy5 labeling reaction were pooled and dried in a speed vac. The pooled samples were then hybridized to the microarray and [symbol] the slides processed according to the general guidelines suggested by the manufacturer (MWG Biotech; High Point, N.C.).

Microarrays were scanned using an Axon 4000B Scanner and GenePix version 4.0 software (Axon; Union City, Calif.) and the resulting image files were quantified as described in Example 1.

An isolated mRNP complex can be examined, in part to determine expression of its components as a whole, or broken down into its individual components. The mRNP complex can be separated from the ligand as a whole, or the mRNA can be separated from the ligand-mRNP complex, followed by separation of the RNA binding protein from the ligand. Alternatively, if the mRNA is bound to the ligand, the RNA binding protein can be separated from the ligand-mRNA complex, and the mRNA then separated from the ligand. Practitioners in [symbol] the art are aware of standard methods of separating the components, including washing and chemical reactions. After separation, each component of an mRNP complex can be examined and their identity, quantity, or other identifying factors preferably recorded (e.g., in a computer database) for future reference.

cDNAs or oligonucleotides can be used to identify complementary mRNAs on mRNP complexes partitioned according to methods disclosed herein. cDNA or oligonucleotide based microarray grids can be used to identify mRNA subsets en masse. Each target nucleic acid examined on a microarray has a precise address that can be located, and the binding can be quantitated. Microarrays may be arranged in a commercially available substrate (e.g., paper, nitrocellulose, nylon, any other type of membrane filter, chip, such as a siliconized chip, glass slide, silicone wafer, or any other suitable solid or flexible support). In addition, mRNAs in a sample can be identified based upon the stringency of binding and washing, a process known as “sequencing by hybridization”, according to standard methods.

Alternative approaches for identifying, sequencing and/or otherwise characterizing the mRNAs in an mRNA subset include, but are not limited to, differential display, phage display/analysis, Serial Analysis of Gene Expression (SAGE), and preparation of cDNA libraries from the mRNA preparation and sequencing of the members of the library.

Methods for DNA sequencing that are well known and generally available in the art may be used to practice any of the embodiments of the invention. The sequencing methods may employ such enzymes as the Klenow fragment of DNA polymerase I, SEQUENASES (U.S. Biochemical Corp, Cleveland, Ohio), Taq polymerase (Perkin Elmer, Boston, Mass.), thermostable T7 polymerase (Amersham, Chicago, Ill.), or combinations of polymerases and proofreading exonucleases such as those found in the Elongase® Amplification System marketed by Gibco BRL (Invitrogen™, Carlsbad, Calif.). Preferably, the process is automated with machines such as the Hamilton Micro Lab 2200 (Hamilton, Reno, Nev.), Peltier Thermal Cycler (PTC200) (MJ Research, Watertown, Mass.) and the ABI Catalyst and 373 and 377 DNA Sequencers (Perkin Elmer, Shelton, Conn.).

In an embodiment, the methods of the invention are carried out on isolated nuclei from cells that are undergoing developmental or cell cycle changes or that have otherwise been subjected to a cellular or an environmental change, performing nuclear run-off assays according to known techniques to obtain transcribing mRNAs, and comparing the transcribing mRNAs with the global mRNA levels isolated from mRNP complexes from the same cells using cDNA microarrays. These methods can distinguish transcriptional from post-transcriptional effects on steady state mRNA levels en masse. As opposed to a total RNA or a transcription profile that depicts RNA accumulation representing a steady-state level of mRNA, which is affected by transcriptional and post-transcriptional events, the mRNAs detected by nuclear run-off experiments represent only the transcription of a gene before the influence of post-transcriptional events. The microarrays representing mRNP complexes contain discrete and more limited subsets of mRNAs than the transcriptome or nuclear run-offs.

Other methods for characterizing and identifying mRNP complex components include standard laboratory techniques such as, but not limited to, RT-PCR, QRT-PCR, RNAse protection, Northern Blot analysis, Western blot analysis, macro- or micro-array analysis, in situ hybridization, immunofluorescence, radioimmunoassay, and immunoprecipitation. The results obtained from these methods are compared and contrasted in order to characterize further the functional relationships of the mRNA subsets and other mRNP components.

The present invention also provides diagnostic methods for assessing the cell types present in a sample or a population of cells such as pancreatic beta cells, adipocytes, preadipocytes, hepatocytes, skeletal muscle, and cardiac muscle. Such analyses can distinguish one cell type from another, cell types of different differentiation states, or cells from one person from another person, for example, a person with a disease or increased risk of disease, from a normal person. The method involves isolating at least one mRNP complex and detecting the expression of at least one component of the mRNP complex, wherein the at least one component is specific for a certain cell type, so that the detection of the expression of the component is indicative of the presence of the cell type in the population of cells. The component may be specific for a certain cell type within an entire sample (e.g., tissue or organism) or within the population of cells. The sample or population of cells may be, for example, a tumor, a tissue, a cultured cell, a body fluid, an organ, a cell extract or a cell lysate. The methods of the invention may also be used to determine the cell types present in a population of cells. Alternatively, cell type, as used herein, may also refer to a class of cells derived from a particular tissue, a particular species, a particular state of differentiation, a particular disease state, or a particular cell cycle.

Validation of Functional Role for Genes Encoding Components of mRNP Complexes

To confirm that a component identified in the an mRNP complex plays a direct role in the etiology of a disease or other phenotype, candidate target genes encoding that component are chosen for gene silencing studies (e.g., using antisense nucleic acids, RNAi, ribozymes, and/or transgenic animals). Comparison of RNA from control RNAi-treated samples with RNA prepared from RNA binding protein RNAi-treated samples can provide quantitative differences in gene expression. Differential expression of genes in samples isolated from RNA binding protein-specific RNAi-treated cells or tissues provides data on identification and quantitative changes in expression due to inhibition of the specific RNA binding protein by RNAi. Genes whose expression patterns are altered as a result of down-regulation of the specific RNA binding protein would be tentatively considered as a member of that RNA binding protein ribonomic cluster.

For example, for each candidate therapeutic gene, one or more short DNA segments representing the coding sequence of that gene is individually cloned into a plasmid vector in the sense or antisense direction, downstream of an appropriate promoter, such as a U6 polymerase III promoter or RNAse P RNA H1. Plasmid vectors may be constructed that contain two or more short DNA segments of one or more candidate therapeutic genes in the sense and antisense directions, downstream of a U6 polymerase III promoter or RNAse P RNA H1. Alternatively, one may construct an RNAi by annealing chemically synthesized complementary 22 bp RNAs (Dharmacon, Lafayette, Colo.).

Following transfection of the vector or double stranded RNA into cultured cells according to standard methods, phenotypic characteristics are evaluated to determine the effect of inhibiting the expression of the candidate target gene(s). In addition, to the inhibition of gene expression at the RNA and protein levels is verified by standard methods, such as, for examples, Northern blots, QRT-PCR, Western blot, or other analytical assay, which may include time course experiments to demonstrate the efficacy and duration of inhibition for the individual genes, according to art known methods.

Transfections can result in transient expression for one to five days. Alternatively, vectors expressing RNAi can be stably expressed in cultured cells by co-transfection and selection with a dominant selectable marker, such as neomycin. As alternatives to the use of RNAi, traditional antisense DNA or vectors expressing dominant negative forms of targets of interest are used. Antisense and dominant negative genes are delivered by direct DNA transfection or through the use of virus vectors including, but not limited to, retroviruses, adenoviruses, adeno-associated viruses, baculoviruses, poxviruses, and polyomaviruses. The biological system of study chosen to demonstrate the role of a gene in disease or cellular phenotype is based upon knowledge in the art of the biological system, including a cell culture or animal model system that mimics relevant biological features.

FIG. 5 illustrates the steps involved in the implementation and validation of RAS™ analysis.

Identification of Therapeutic Targets

The invention provides methods for identifying a therapeutic target by comparing the ribonomic profiles of a “test” cell sample (e.g., a cell that has been treated with an agent or is derived from a diseased individual) to the ribonomic profiles of a control sample (e.g., a cell that is untreated or derived from a non-diseased individual). A difference in the expression of a component of an mRNP complex between the two samples is indicative that the component is regulated by, or regulates, other components of the mRNP complex and that therefore it is a candidate therapeutic target (e.g., for the up or down-regulation of that component or a component that it regulates). The therapeutic target may include, but is not limited to, any component of an mRNP complex, nucleic acid coding therefore, or gene product thereof. In an embodiment of the invention, the test cell sample is treated with a test compound and the control sample comprises cells that have not been treated with the test compound. In another embodiment, the test and control cell samples comprise cells at different stages in their growth cycle. In yet another embodiment, the test cell sample comprises a tumor cell or other diseased cell, and the control sample comprises a normal cell. Target identification includes methods known to practitioners in the art, such as, but not limited to, the use of screening libraries, peptide phage display, cDNA microchip array screening, and combinatorial chemistry techniques known to practitioners in the art. Once the mRNA or protein target has been identified, its role in a particular physiological pathway or process is assessed. For example, an mRNA or protein can be inhibited or overexpressed in a cell or organism according to standard methods. The effect of the under- or Over-expression can then be assessed by phenotypic analysis of the cell or organism. For example, RNAi may be used to knock out gene expression of the component. The gene expression of other components of the physiological pathway can be assessed, for example, using microarrays, in order to determine the regulatory effect of the altered target on other components of the process or pathway. A summary of the steps for target discovery is provided in FIG. 5.

Identification of Therapeutics

In another aspect, the invention provides methods for assessing the efficacy of a test compound as a therapeutic. A cell sample is contacted with a test compound and a ribonomic profile of the cell sample comprising the expression of at least one gene product associated with at least one mRNP complex is prepared. The expression levels of the gene product(s) in the cell sample are compared to the expression levels of the gene product(s) in a control sample (e.g., a cell sample that is not contacted with a test compound). Identification of a difference in expression of the gene product between the treated and untreated cell samples is indicative that the test compound is a potential therapeutic. Test compounds may be, for example, nucleic acids, hormones, antibodies, antibody fragments, antigens, cytokines, growth factors, pharmacological agents (e.g., chemotherapeutics, carcinogenics, or other cells), chemical compositions, proteins, peptides, and/or small molecules.

In various embodiments of the invention, the therapeutic may stabilize or destabilize the mRNA or the mRNP complex-associated protein. In another embodiment, the therapeutic may either inhibit or enhance translation of the mRNA, inhibit or accelerate transport of the mRNA or the mRNP complex-associated protein, inhibit the binding of the RNA binding protein to the mRNA, inhibit the binding of the RNA binding protein to the mRNP complex-associated protein, or inhibit the binding of the mRNA to the mRNP complex-associated protein, for example.

In another aspect, the invention provides methods for assessing toxicity, potential side effects, specificity or selectivity of a test compound, for example, by altering the concentrations or amounts of a test compound used to treat a cell sample.

In yet another aspect, the present invention provides methods for monitoring the efficacy of a therapeutic in a subject. In accordance with the invention, an effective amount of a therapeutic is administered to a subject. At least one mRNP complex is isolated from a cell sample from the subject, wherein altered expression of a gene product associated with the mRNP complex is altered by administration of the therapeutic. The expression of the gene product in the cell sample after administration of the therapeutic is compared to the expression of the gene product in a control sample (e.g., a second cell sample obtained from the subject either prior to administration of the therapeutic or from a normal subject). The tests are repeated over a period of time to monitor the continued efficacy of the therapeutic. A difference in expression between the treated and the control cell samples is indicative of the efficacy of the therapeutic.

Therapeutics may target over- or under-expressed proteins involved in the etiology of a disease, disorder, or condition. Such over- or under-expression may result in destabilization or stabilization of RNA and/or inhibit or enhance translation of the substrate RNA.

Therapeutics that Destabilize mRNA

If a disease, condition or disorder is characterized by overexpression of a protein, a therapeutic for treatment of such a condition will reduce or eliminate expression of the protein by decreasing the stability of the RNA encoding the protein and/or by inhibiting the translation of the RNA. For example, since RNA binding proteins enhance the stability of short-lived mRNAs encoding protooncogenes, growth factors and cytokines that contribute to cell proliferation, inhibition of RNA binding protein production may alleviate diseases such as cancers or autoimmune diseases (e.g., by decreasing tumor growth or inflammation, respectively). In addition, RNA binding protein overexpression in several human tumors correlates with resistance to chemotherapy and UV irradiation. Increased stability of c-fos, c-myc, cyclin B1 and other short-lived mRNAs in response to UV-irradiation or therapeutic drugs is well known. Accordingly, inhibition of RNA binding protein expression in these tumors destabilizes the mRNA in the tumors and, as a result, renders the tumors more responsive to cancer treatments.

In order to reduce overexpression or to cease expression of a protein of interest, the mRNA can be destabilized or its translation inhibited by administering an effective amount of a suitable test compound (e.g., an RNA binding protein inhibitor) either in vitro or in vivo. The test compound may bind mRNA so as to inhibit RNA binding protein binding to the mRNA by binding to the RNA binding protein, bind to and destabilize the mRNP complex, and/or bind the mRNA so as to directly destabilize or inhibit the translation of the mRNA, and/or bind the RNA binding protein so as to inhibit the translation of the mRNA, for example. Compounds that bind to the mRNA but that do not stabilize the mRNA may inhibit the ability of an RNA binding protein to stabilize the mRNA or regulate translation of the mRNA. If the compound binds competitively with an RNA binding protein, the compound can decrease mRNA stability by inhibiting the RNA binding protein's ability to bind the mRNA.

Alternatively, the test compound may inhibit RNA binding protein expression or its mRNA expression.

Effective test compounds (e.g., RNA binding protein inhibitors) can be readily determined by screening compounds for their ability to interfere with the production of RNA binding protein or their ability to inhibit the binding to, and/or stabilization or translation of, mRNA, for example, by methods described herein. Compounds that function by inhibiting RNA binding protein or mRNA production can be identified by exposing cells that express the RNA binding protein or mRNA of interest and monitoring the levels of RNA binding protein or mRNA expressed, respectively. Compounds that function by inhibiting the stabilizing effect of an RNA binding protein and/or its ability to inhibit translation of an mRNA can be identified by combining RNA binding protein and an mRNA that would otherwise be stabilized, adding compounds to be evaluated as RNA binding protein inhibitors, or compounds that enhance RNA binding protein to result in inhibition of translation and monitoring the binding affinity of RNA binding protein and the mRNA. Compounds that increase or decrease the binding affinity of RNA binding protein and the mRNA can be readily determined by art known methods.

Therapeutics that Stabilize mRNA

If a disease, condition or disorder is characterized by underexpression of an mRNA stabilizing protein or results from inhibited translation of the mRNA, a therapeutic for treatment of such a medical condition may operate by stabilizing the mRNA associated with the underexpressed protein and/or enhancing the translation of the mRNA. Accordingly, mRNA may be stabilized or its translation enhanced by administering an effective amount of a compound, either in vitro or in vivo. The compound may possess a similar binding ability and stabilizing and/or translation enhancing effect as the RNA binding protein or, may promote the RNA binding protein's ability to stabilize and/or enhance the translation of the mRNA, and/or may promote the production of the RNA binding protein or the mRNA of the RNA binding protein of interest. Such a compound may be referred to as an RNA binding protein inducer and may operate by interacting with the mRNA, the RNA binding protein or both. Alternatively, mRNA can be stabilized and/or its translation enhanced by administering an effective amount of a suitable RNA binding protein that possesses the necessary mRNA stabilizing and/or translation enhancing effect.

Compounds that increase RNA binding protein production can be identified by initially exposing cells that express the RNA binding protein to potential inducers and, monitoring the levels of the RNA binding protein, in accordance with the methods described above. If the level of RNA binding protein expression increases, the compound is an RNA binding protein inducer. Compounds that inhibit RNA binding protein binding to mRNA, but which bind and stabilize and/or enhance translation of the mRNA, can be identified by methods disclosed herein. A skilled practitioner may combine RNA binding protein and an mRNA, add a compound, and monitor the binding affinity of the RNA binding protein and the mRNA. Compounds that increase or decrease the binding affinity of an RNA binding protein and the mRNA can be readily determined by evaluating the binding affinity of the RNA binding protein to the mRNA after exposure to the compound, as described herein. By monitoring the concentration of mRNA and/or translation of mRNA over time, those compounds that bind to the mRNA can then be assayed for their ability to stabilize and/or enhance translation of the mRNA.

High Throughput Screening Methods for Libraries of Compounds

In an embodiment of the invention, high throughput screening assays and competitive binding assays are used to identify compounds that bind to an mRNP complex or component thereof from combinatorial libraries of compounds (e.g., phage display peptide libraries, small molecule libraries and oligonucleotide libraries).

In one embodiment, an mRNP component, catalytic or immunogenic fragment thereof, or oligopeptide thereof, can be used to screen libraries of compounds in any of a variety of drug screening techniques. An exemplary technique is described in published PCT application W084/03584, hereby incorporated by reference. The fragment employed in such screening can be free in solution, affixed to a support, or located on a cell surface or intracellularly.

The SELEX method, described in U.S. Pat. No. 5,270,163, is used to screen oligonucleotide libraries for compounds that have suitable binding properties. In accordance with the SELEX method, a candidate mixture of single stranded nucleic acids with regions of randomized sequence can be contacted with the mRNP complex. Those nucleic acids having an increased affinity to the mRNP complex can be partitioned and amplified so as to yield a ligand enriched mixture.

Phage display technology is used to screen peptide phage display libraries to identify peptides that bind to an mRNP complex or component thereof. Methods for preparing libraries containing diverse populations of various types of molecules such as peptides, polypeptides, proteins, and fragments thereof are known in the art. Phage display libraries are also commercially available.

A library of phage displaying potential binding peptides is incubated with an mRNP complex to select clones encoding recombinant peptides that specifically bind the mRNP complex or components thereof. After at least one round of biopanning (binding to the mRNP complex), the phage DNA is amplified and sequenced, thereby providing the sequence for the displayed binding peptides. Briefly, the target, an mRNP complex, can be coated overnight onto [symbol] tissue culture plates and incubated in a humidified container. In a first round of panning, approximately 2×10¹¹ phage can be incubated on the protein-coated plate for 60 minutes at room temperature while rocking gently. The plates are then washed using standard wash solutions. The binding phage can then be collected and amplified following elution using the target protein. Secondary and tertiary pannings can be performed as necessary. Following the last screening, individual colonies of phage-infected bacteria can be picked at random, the phage DNA isolated and subjected to automated dideoxy sequencing. The sequence of the displayed peptides can be deduced from the DNA sequence.

The biological activity of compounds can be evaluated using in vitro assays known to those skilled in the art (e.g., protein synthesis assays or tumor cell proliferation assays). Alternatively, the biological activity of the compounds is evaluated in vivo. Various compounds including antibodies, can bind to mRNP complexes and components thereof with varying effects on mRNA stability. The activity of the compounds once bound can be readily determined using the assays described herein.

Binding assays include cell-free assays in which an RNA binding protein and an mRNA are incubated with a labeled test compound. Following incubation, the mRNA, free or bound to a test compound, can be separated from unbound test compound using any of a variety of techniques known in the art. The amount of test compound bound to an mRNP complex or component thereof is then determined, using detection techniques known in the art.

Alternatively, the binding assay is a cell-free competition binding assay. In such assays, mRNA is incubated with labeled RNA binding protein. A test compound is added to the reaction [symbol] and assayed for its ability to compete with the RNA binding protein for binding to the mRNA. Free labeled RNA binding protein can be separated from bound RNA binding protein. By subsequently determining the amount of bound RNA binding protein, the ability of the test compound to compete for mRNA binding can be assessed. This assay can be formatted to facilitate screening of large numbers of test compounds by linking the RNA binding protein or the mRNA to a support so that it can be readily washed free of unbound reactants. A plastic support (e.g., a plastic plate such as a 96 well dish or chip) is preferred. The RNA binding protein and mRNA suitable for use in the cell-free assays described herein can be isolated from natural sources (e.g., membrane preparations) or prepared recombinantly or chemically. The RNA binding protein can be prepared as a fusion protein using, for example, known recombinant [symbol] techniques. Preferred fusion proteins include, but are not limited to, a glutathione-S-transferase (GST) moiety, a green fluorescent protein (GFP) moiety that is useful for cellular localization studies or a His tag that is useful for affinity purification.

A competitive binding assay may also be cell-based. Accordingly, a compound, preferably labeled, known to bind an mRNP complex or component thereof, is incubated with the mRNP complex or component thereof in the presence and absence of a test compound. By comparing the amount of known test compound associated with cells incubated in the presence of the test compound with that of cells incubated in the absence of the test compound, the affinity of the test compound for the RNA binding protein, mRNA, and/or complex thereof can be determined. Cell proliferation can be monitored by measuring the uptake into cellular nucleic acids of labeled bases (e.g., radioactively, such as ³H, SiC, or ¹⁴C; fluorescently, such as CYQUANT (Molecular Probes, Eugene, Oreg.); or colorimetrically such as BrdU (Sigma, St. Louis, Mo.) or MTS (Promega, Madison, Wis.)) as known in the art. Cytosolic/cytoplasmic pH determinations can be made with a digital imaging microscope using substrates such as bis(carboxyethyl)-carbonyl fluorescein (BCECF) (Molecular Probes, Inc., Eugene, Oreg.).

Other types of assays that can be carried out to determine the effect of a test compound on RNA binding protein binding to mRNA include, but are not limited to, the Lewis Lung Carcinoma assay and extracellular migration assays such as the Boyden Chamber assay.

Accordingly, the methods permit the screening of compounds for their ability to modulate the effect of an RNA binding protein on the binding of and stability of mRNA. Using the assays described herein, compounds capable of binding to mRNA and modulating the effects on those cellular bioactivities resulting from mRNA stability and correlated protein synthesis are identified. The compounds identified in accordance with the above assays are formulated as therapeutic compositions.

Diagnosing and Monitoring Disease

In another aspect, the invention provides methods for diagnosing a disease or risk of a disease related to glucose and/or lipid metabolism (e.g., obesity or diabetes) or cellular function. A ribonomic profile from a subject's cell sample is prepared and at least one mRNP complex is analyzed. The expression of at least one gene product, for which altered expression is indicative of a disease or risk of disease, is determined. The gene product may be an RNA binding protein, an mRNA, an mRNP complex-associated protein or other gene product bound to or associated with the mRNP complex. The expression of the gene product in the cell sample is compared to the expression of the gene product in a control sample. The control sample may be, for example, a sample of normal cells or a second cell sample from the subject. Alternatively, the control sample is a positive control, for example, from a diseased and/or normal individual. By observing the relative expression of the gene product in the cell sample compared to the control sample, the presence of a disease or risk of disease can be determined.

In another aspect, the invention discloses a method for monitoring a disease state in a subject. At least one mRNP complex is isolated from a diseased subject's cell sample, wherein the mRNP complex has at least one gene product that is associated with the disease. The expression of the gene product in the subject's cell sample is compared to the expression of the gene product in a control sample. The identification of a difference in the expression of the gene product in the diseased subject cell sample compared to the expression of the gene product in the control sample is indicative of a change in the disease state of the subject. For example, a decrease in the production of a tumor related antigen or its mRNA is indicative of decreased tumor load or remission; by contrast, an increase in expression of the tumor antigen is indicative of aggressive tumor growth. Such monitoring during drug treatment provides information about the effectiveness of the subject's drug regimen, and may indicate when a particular regimen is not, or is no longer, effective for treating the disease or condition. The control sample may be, for example, a second cell sample from the subject, preferably, obtained when the subject is free of one or more symptoms of the disease. Alternatively, the control sample is, for example, from a normal subject or other normal cell sample.

In summary, the present invention provides useful in vivo and in vitro methods for determining the ribonomic profile of a cell and detecting changes in the ribonomic profile. The invention has numerous uses, including, but not limited to, monitoring cell development or growth, monitoring a cell state, and monitoring perturbations of a biological system such as disease, condition or disorder. The invention further provides methods for diagnosing a disease, condition, or disorder and determining appropriate treatment regimens. The invention also is useful for distinguishing ribonomic profiles among organisms such as plant, fungal, bacterial, viral, protozoan, or animal species.

The present invention can be used to discriminate between transcriptional and post-transcriptional contributions to gene expression and to track the movement of RNAs through mRNP complexes, including the interactions of combinations of proteins with RNAs in mRNP complexes. Accordingly, the present invention can be used to study the regulation of RNA stability. The present invention can be used to investigate the activation of translation of mRNAs as single or multiple species by tracking the recruitment of mRNAs to active polysomes [symbol] measuring the sequential, ordered expression of mRNAs such as mRNAs that encode transcription factors or RNA binding proteins, and measuring the simultaneous, coordinate expression of multiple mRNAs. The present invention can also be used to determine the transacting functions of RNAs themselves upon contacting other cellular components. These and numerous other uses will be made apparent to the skilled artisan upon study of the present specification and claims.

The following Examples are set forth to illustrate the present invention, and are not to be construed as limiting thereof.

### EXEMPLIFICATION

### Example 1

### Target Discovery Using Ribonomic Profiles

The general steps required for target discovery using the methods of the invention are summarized in FIG. 5. Briefly, expression profiles for RNA binding proteins are generated to identify RNA binding proteins that have altered expression in different cell types, in a disease phenotype, or in response to certain stimuli, for example. Candidate RNA binding proteins may then be cloned and their cDNAs inserted into various bacterial and mammalian expression vectors for production of recombinant RNA binding proteins and overexpression of RNA binding proteins, respectively. Recombinant or purified RNA binding proteins are then used to generate monoclonal or polyclonal antibodies for use in RAS™ analysis performed on extracts from cells or tissues. Intact mRNP complexes associated with the differentially expressed RNA binding protein are then immunoprecipitated, for example, using antibodies to the RNA binding protein. Once the mRNP complex is isolated, the other components of the mRNP complex, including RNAs and other mRNP complex associated proteins, are identified and compared and characterized. Differential expression of the other components of the mRNP complex is determined in different cell types, in a disease phenotype, or in response to certain stimuli. Once [symbol] differential expression is determined and candidate mRNP components are identified, their biological role, e.g., participation in a certain pathway or disease, is validated by inhibition and overexpression studies. mRNP components that participate in a certain pathway are candidate therapeutic targets for diseases relating to aberrant regulation of that pathway.

Establishing Expression Profiles for RNA Binding Protein Genes

In one procedure for identifying candidate RNA binding proteins for further analysis, RNA binding protein expression profiles are generated in control or agent treated cell lines or tissues, and from normal and diseased human tissues. The agents used to treat the cells or tissues may include any agent that affects insulin action, insulin secretion glucose metabolism or lipid metabolism such as, adiponectin, leptin, resistin (or agents that act through the receptors for adiponectin, leptin, resistin), tumor necrosis factor-alpha, glucose, insulin, a beta-adrenergic agonist, insulin-like growth factor-1 (IGF-1), glucagon-like peptide-1 (GLP-1), fatty acid, peroxisome proliferator activated receptor (PPAR) ligands (e.g. thiazolidinediones, fibrates, halogenated fatty acids, and tyrosine derivatives), insulin-like growth factor-2 (IGF-2), RNAi against a RNA binding protein, an agent that enhances RNA binding protein expression and/or a small molecule (e.g., putative drug).

Initial tissue, disease, or agent screening of RNA binding protein gene expression can be accomplished by Quantitative Real Time PCR (QRT-PCR) using oligo dT-primers and commercially available RNA samples (Stratagene, Inc., La Jolla, Calif.; Ambion, Inc., Austin, Tex.; BD Biosciences Clontech, Palo Alto, Calif.). 10-100 μg of cDNA is used to perform Quantitative PCR (Q-PCR) using SybrGreen (Molecular Probes, Inc., Eugene, Oreg.) and gene specific PCR primers on a BioRad iCycler Quantitative PCR machine (Biorad, Hercules, Calif.) using protocols provided by the manufacturer. Experimental results are analyzed using the accompanying BioRad iCycler software. RNA levels for candidate RNA binding proteins are normalized to rRNA.

In addition to the above approaches, for rapid and comprehensive screening of tissues and cell lines, a RIBOCHIP™ array (Ribonomics, Inc., Durham, N.C., designed and manufactured by MWG Biotech USA, Highpoint, N.C.) may be used. The RIBOCHIP™ contains 50-mer oligonucleotides corresponding to RNA binding protein genes in duplicate, non-contiguous positions, plus control genes, on glass slides. The nucleic acid sequences were compiled from a wide variety of public databases and search tools including GenBank (NCBI, Bethesda, Md.), PubMed (NCBI, Bethesda, Md.), SRS Evolution (LION Biosciences, Cambridge, Mass.), LocusLink (NCBI, Bethesda, Md.), Protein FAMily database (pFAM, Washington University, St. Louis, Mo.); Welcome Institute; Sanger Institute (Hinxton, UK), GO Database (Gene Ontology™ Consortium, Gene Ontology: tool for the unification of biology. The Gene Ontology Consortium (2000) Nature Genet. 25: 25-29), Structural Classification of Proteins (SCOP©), and Package (Medical Research Council, Cambridge, UK). A detailed method for microassay analysis on the RIBOCHIP™ and section of differentially expressed genes is described below.

The RNA binding proteins identified as having altered expression in response to treatments, disease, or cell cycle changes are useful for prioritizing candidates for RAS™. In addition, RNA binding proteins themselves may be candidates for therapeutic targeting and/or gene therapy (i.e., gene replacement or gene silencing) or therapeutic antibody targets.

Cloning and Expression of RNA Binding Protein Genes in Bacterial Vectors

When candidate RNA binding proteins are identified, full length cDNA clones are generated by reverse transcriptase-PCR (RT-PCR) using commercial RNA tissue sources and standard methods. For example, full-length plasmid clones are constructed based on phage lambda-based (att) site-specific recombination protocols (Invitrogen, Corp., Carlsbad, Calif.) for the GATEWAY™ pENTRD-Topo entry vectors and pDEST17 6XHis destination vectors (Invitrogen, Corp., Carlsbad, Calif.) or glutathione S transferase vectors (e.g., pGEX from Amersham, Piscataway, N.J.). Escherichia coli (e.g., BL21SI or BL21A1) expressing polyhistidine-tagged or GST-tagged RNA binding protein fusion proteins are grown to mid-log phase at 37° C. and induced in 0.3 M NaCl for BL21SI cells or in 0.2% mM arabinose or about 0.1 mM to about 1 mM IPTG for BL21A1 cells at 20-37° C. for about 2-6 hours (specific time based upon optimization in pilot expression studies for each clone). Bacterial cells are lysed by sonication and the RNA binding protein-fusion protein is purified on nickel columns (Qiagen, Inc., Valencia, Calif.) or glutathione Sepharose (Amersham, Piscataway, N.J.) using standard methods. Insoluble fusion proteins are maintained and purified in the presence of 8M urea, and soluble proteins are maintained in phosphate buffered saline (PBS). The purified fusion proteins are used for immunization of mammals (e.g., rabbits, pigs, or chickens) for production of polyclonal antibodies using standard methods. Polyclonal antibodies are characterized by their ability to immunoprecipitate and detect by western blot, for example, native and recombinant proteins. The recombinant RNA binding protein is also used for in vitro RAS™ described below.

Analysis of Other mRNP Complex Components

Changes in the abundance or constellation of RNA binding proteins in a cell affect the processing of any mRNAs bound to those RNA binding proteins. The subset of mRNAs that are associated with an RNA binding protein is indicative of functional co-regulation that is critically or causally involved in effecting a phenotypic change in the cell. Thus, those genes whose mRNAs are associated with tissue-, disease-, or agent altered mRNP complexes are a rich source of potential therapeutic targets.

RNA binding proteins that exhibit the most dramatic variation with regard to expression proceed into the next stage of analysis, the Ribonomic Analysis System (RAS™) assay (Ribonomics, Durham, N.C.). The RAS™ assay uses a microarray format to identify and/or quantify the specific mRNAs associated with particular RNA binding proteins. Commercially available glass slide arrays (such as, for example, Human Unigene 14K, Agilent, Palo Alto, Calif. and Pan Human 10K, MWG Biotech, Inc., High Point, N.C.), or membrane arrays, such as, for example, ATLAS™ Arrays, BD Biosciences, Clontech, Palo Alto, Calif.), are employed using protocols for hybridization, washing, and development provided by the array manufacturers.

The composition of RAS™ assay lysis buffer (RLB) may vary, depending on the binding characteristics of a particular RNA binding protein. Basic RLB contains 50 mM HEPES, pH 7-7.4, 1% NP-40, 150 mM NaCl, 1 mM DTT, 100 U/ml RNase OUT (Gibco BRI, Invitrogen Corp., Carlsbad, Calif.), 0.2 mM PMSF (Sigman Aldrich, St. Louis, Mo.), 1 μg/ml aprotinin (Sigman Aldrich, St. Louis, Mo.) and 1 ug/ml leupeptin (Sigman Aldrich, St. Louis, Mo.). Variations of these basic components included changes in salt concentrations (e.g., about 0 to about 500 mM NaCl or about 0 to about 5 mM KCl), ionic conditions (about 0 to about 10 mM MgCl₂ or about 0 to about 20 mM EDTA), and reducing environment (about 0 to about 5 mM DTT). For example, in order to prepare cell extracts for examining the polypyrimidine tract binding protein (PTB) mRNP complex, cultured cells are washed in ice-cold PBS and scraped directly into RLB containing 5 mM MgCl₂ and incubated on ice for 10 minutes followed by centrifugation at 3,700×g for 10 minutes at 4° C.

It is necessary in certain cases to crosslink the mRNP complex prior to isolation so that the RNA binding protein remains associated to its mRNAs. This is performed on cultured cells as well as fresh tissue samples. The extent of crosslinking is titrated for each cell line or tissue and monitored based on the ability to immunoprecipitate mRNA in the complex. For example, cultured cells or tissues are incubated in PBS containing about 0 to about 1% formaldehyde at room temperature for about 15-60 minutes. Crosslinking is then quenched by the addition of 1M Tris pH 8.0 to a final concentration of 250 mM Tris pH 8.0 and incubated further for an additional 20 minutes. The samples are then washed 3× in PBS containing 50 mM Tris pH 8.0. For cultured cells, the cells are pelleted and resuspended in radioimmunoprecipitation (RIPA) buffer (50 mM Hepes, pH 7.4, 150 mM NaCl, 1% NP-40, 0.1% SDS, 0.5% deoxycholate (DOC) (Sigma-Aldrich, St. Louis, Mo.) and 100 U/ml RNase Out (Gibco BRI, Invitrogen Corp., Carlsbad, Calif.) to about 2 mg/ml final protein concentration. For tissues, the samples are resuspended in RIPA and homogenized with a polytron to disrupt the tissue. Following the initial lysis, the samples are subjected to sonication with a probe sonicator (Branson 450, Branson Ultrasonics Corp., Danbury, Conn.) at output setting 6, two times for 20 seconds each. Between sonications the samples are allowed to cool on ice for 2 minutes. Lysates are then cleared by centrifugation at 3,700×g for 15 minutes. The next stages include immunoprecipitation and RNA extraction.

Immunoprecipitation of mRNP Complexes and RNA Extraction

On average, typical final protein concentrations for the cellular lysates are 2 mg/ml. Approximately 2 mg protein is used for each immunoprecipitation condition. Cleared cellular extracts are incubated with primary antibody (e.g., an anti-PTB (Zymed, South San Francisco, Calif.) is used at a final concentration of 10 μg/ml) or a control antibody at equal concentration (e.g., pre-immune or IgG sera (Pierce Biotechnology, Rockford, Ill.) at final concentration of 10 μg/ml) for 2 hours at 4° C. A 25 μl aliquot of Protein A Trisacryl beads (Pierce Biotechnology, Rockford, Ill.) is added and the samples rotated for 1 hour at 4° C. The immune complex is then washed 6× in RLB buffer by adding 1 ml of RLB buffer followed by brief centrifugations in a microcentrifuge for 30 seconds at 5,000 rpm. After the final wash, 50 μl of RNA extraction buffer from the PICOPURE™ RNA isolation kit (Arcturus, Inc., Mountain View, Calif.) is added to the beads, vortexed briefly and centrifuged to pellet the beads. The extracted RNA is purified following the PICOPURE™ protocol (Arcturus, Inc., Mountain View, Calif.). RNA present in the mRNP complex is then quantified using the RIBOGREEN™ assay (Molecular Probes, Inc., Eugene, Oreg.).

Amplification of RNA for Microarray Analysis

Since mRNA isolated from mRNP complexes represents only a small subset of total RNA, isolated mRNA may be amplified prior to labeling. Message Amp™ (Ambion, Inc., Austin, Tex.) is used for RNA amplification according to the manufacturer's instructions. Two rounds of amplification are performed prior to labeling by random primer polymerization with Cy3 or Cy5-dUTP. Hybridization and washing are performed according to the microarray manufacturer's protocols and as described above. Microarray data acquisition and analysis are performed as described below.

Microarray Analysis

These methods are employed for analysis of RNA for ribonomic profiling with the RIBOCHIP™ as well as analysis on pan arrays with RNA extracted from the mRNP complexes to identify genes within a Ribonomics cluster.

RNA Preparation

The mRNA samples to be analyzed are prepared from various cell and tissue-types by RNA extraction with RNeasy™ (Qiagen, Inc.), quantified by absorbance (A₂₆₀), and stored at −80° C. until use. Purified, Dnase I treated RNA was routinely analyzed using an Agilent 2100 Bioanalyzer. RNA was assessed for purity by examining electropherograms for the presence of broad peaks overlapping the 28S and 18S ribosomal RNA (rRNA) peaks. Broad peaks of this nature indicate contamination with genomic DNA. If such contamination was detected, the RNA was retreated with Dnase I and purified as described above. In addition, the relative abundance of 28S to 18S rRNA was determined to assess the quality of the RNA sample. Ratios greater than or equal to about 1.7 for 28S/18S rRNA indicate little or no degradation of the RNA and are acceptable for microarray analysis. Ratios less than about 1.7 indicate degraded RNA that is not acceptable for microarray analysis.

Synthesis of Aminoallyl-UMP Labeled cDNA

Aminoallyl cDNA was synthesized based on modifications of protocols by DeRisi (www.microarray.org; “Reverse Transcription and aa-UTP Labeling of RNA”) and TIGR (www.tigr.org; Protocol M005). Briefly, total RNA (10 μg) was combined with 2 μl dT₁₈ (200 μM), 2 μl random decamer (1 mM stock), and diethyl pyrocarbonate (DEPC) treated water to a final volume of 17.5 μl. Primers were annealed to the RNA template by heating at 70° C. for 10 minutes and then cooling to room temperature or on ice. Aminoallyl cDNA was synthesized by addition of combining the above reaction with 6 μl SuperScript II first strand buffer, 3 ml 0.1 M dithiothreitol, 0.6 ml 50× labeling mix (25 mM dATP, 25 mM dGTP, 25 mM dCTP, 15 mM dTTP, and 10 mM aminoallyl-dUTP (Sigma; St. Louis, Mo.; Catalog A0410)), 1 ml RNAseOUT (Invitrogen; Carlsbad, Calif.; Catalog 10777-019), and 1 ml SuperScript II (Invitrogen; Carlsbad, Calif.; Catalog 18064-022) followed by incubation for 3 to 24 hours at 42° C. The RNA was hydrolyzed by addition of 10 μl each 1 M NaOH and 0.5 M ethylenediamine tetraacetic acid followed by incubation for 15 minutes at 65° C. The solution was neutralized by addition of 10 μl of 1 M HCl. The aminoallyl-cDNA was purified using Qiagen QiaQuick PCR purification kit with the following modifications. The cDNA was mixed with 5× reaction volumes of the Qiagen supplied PB buffer and transferred to a QIAquick column. The column was placed in a collection tube and centrifuged for 1 minute at 13,000 rpm. The column was washed by addition of 750 μl of phosphate wash buffer (prepared by mixing 0.5 mL 1 M KPO₄ (9.5 mL 1M K₂HPO₄+0.5 mL 1M KH₂PO₄), pH 8.5; 15.25 RNase free water; and 84.25 mL 95% ethanol) and centrifuging at 13,000 rpm. The wash step was repeated and the column centrifuged 1 minute at maximum speed to remove all traces of wash solution. The column was transferred to a clean collection tube and the aa-cDNA was eluted by addition of 30 μl of phosphate elution buffer (prepared by mixing 0.5 mL 1 M KPO₄, pH 8.5; 15.25 RNase free water; and 84.25 mL 95% ethanol). The elution was repeated once and the sample was dried in a speed-vac.

Coupling of Cyanin Reactive Esters to aa-CDNA and Purification of Labeled cDNA

The purified aa-cDNA was coupled to cyanine dyes (Amersham Biosciences; Piscataway, N.J.; Catalog # PA23001 (Cy3) or PA25001 (Cy5)); purified; and analyzed as described. Stock solutions of Cyanin3 and Cyanin5 reactive N-hydroxysuccinamide dye were prepared by dissolving one tube of reactive dye in 73 μl of anhydrous DMSO. Reactive dye was coupled to aa-cDNA by addition of 4.5 μl reactive DMSO dye solution to the aa-cDNA and incubating for 1 hour in the dark at room temperature. Following coupling, the dye-labeled cDNA was purified using standard QIAquick PCR cleanup kit methods and buffers. The labeling reactions were analyzed for incorporation according the TIGR M005 protocol.

Hybridization and Processing of Spotted Microarrays

Each spotted microarray is sufficient for analysis of two Cy-dye labeled samples, one labeled with Cy3 and one labeled with Cy5. For each microarray, material from one Cy3 labeling and one Cy5 labeling reaction were pooled and dried in a speed vac. The pooled samples were then hybridized to the microarray and the slides processed according to the general guidelines suggested by the manufacturer (MWG Biotech, High Point, N.C.).

Microarray Data Extraction and Analysis

FIG. 6 provides a flow chart of the data extraction and analysis using microarrays. Microarrays were scanned using an Axon 4000B Scanner and GenePix version 4.0 software (Axon, Union City, Calif.). The resulting image files were quantified using BioDiscovery's Imagene software version 5.5 (El Segundo, Calif.) using standard background and spot finding settings. Two methods of data analysis were employed. The preferred method involved pre-processing the data using the BioConductor Suite (www.bioconductor.org; v 1.2) of microarray libraries for the R statistical environment (www.r-project.org; v 1.7.1). Preprocessing involved background subtraction, application of intra-array Lowess intensity and location dependent normalization, and, in some cases, inter-array scaling using the MAD function of the BioConductor normalization library. The normalized intensity data was exported for further analysis in GeneSpring (Silicon Genetics; Redwood City, Calif.). Within GeneSpring, differentially expressed genes were identified based on ANOVA analysis (Welch's t-test for 2 conditions) and a suitable p-value threshold. Typically, a p-value of ≦0.05 was employed, although this value could be increased as necessary. Additionally, one or more of the available multiple testing corrections were applied to the data to reduce the occurrence of false positives. This was not always possible, particularly if the number of replicates available was too small. An alternative and less desirable method of data analysis was also employed occasionally. This involved filtering the data based on background subtracted signal intensity (e.g. ≧500) and fold differential expression between the experimental and control samples (e.g. ≧2 fold differential from control). Routinely, genes expressed at a level above local background are considered members of that cluster. The presence of the candidate genes and their relative folds enrichment over total RNA is verified and more accurately quantified by a QRT-PCR using sequence-specific primers.

In a standard RAS™ analysis (e.g., comparing normal vs. disease cells or treated vs. untreated cells), quantitative and qualitative changes in the total RNA content are compared to changes in the RNA content of the particular mRNP complex. The data obtained is routinely grouped into four classes: (1) RNAs that show comparable quantitative changes in the mRNP complex, (2) RNAs present in the total RNA but not in the mRNP complex, (3) RNAs present in the mRNP complex but apparently absent or below the level of detection in total RNA, and (4) RNAs that change in the cluster in a quantitatively different manner than in the total RNA analysis. In addition, the RAS™ assay identifies genes represented by class 4 that do not change in total abundance but that are repartitioned within the cell for alternative processing and regulation. As a result, different splice variants may be translated, the mRNA might be transported to and translated at a specific location within the cell, or translation itself might be up or down modulated. The subsets of genes identified within groups 3 and 4 cannot readily be identified by any other currently available approach to characterization of gene expression.

The methods of the invention identify genes that participate in the cellular pathways that contribute to the phenotypic changes associated with disease or certain cellular states and thus are attractive therapeutic targets. In addition, the methods of the invention identify target classes that have proven to be tractable targets for small molecule drugs. These target classes include nuclear receptors (e.g., hormone receptors), G-protein coupled receptors, phosphodiesterases, kinases, proteases, and ion channels, among others. Other target classes of therapeutic interest include secreted molecules, extracellular ligands, and phosphatases.

For RNA binding proteins identified or differentially expressed on the RIBOCHIP™ and for candidate target genes or gene products identified by the RAS™ assay followed by global gene expression analysis on pan arrays, QRT-PCR was used to validate the expression at the RNA level when possible at the protein level by Western blot. For QRT-PCR, RNA is reverse transcribed to cDNA using Superscript II reverse transcriptase (Invitrogen, Carlsbad, Calif., Cat# 18064-014) following the recommended kit protocol.

In 96 well PCR plates, 50 ng of cDNA/well were incubated with 1× iQ sybr green supermix (Biorad, Hercules, Calif. Cat# 94547) and either reaction specific or control primer pairs for a final volume of 50 ul. All reactions were in duplicate. QRT-PCR reactions were run on a Biorad iCycler machine, using the sybr 2 step program (1 cycle at 95 C for 8 minutes and 30 seconds; 40, 2 step cycles of 95 C for 30 seconds followed by 60 C for 60 seconds; 100 cycles of 55 C for 10 seconds). Data are compared to a normalized gene such as actin, GAPDH, or ribosomal RNA. Differences in cycle time are used to compare and determine expression values relative to controls.

Immunoprecipitation of RNA Binding Protein Complexes

As an example of immunoprecipitation and isolation of a mRNP complex using RAS™, the PTB ribonomic cluster (referred to also as PTB-cluster or PTB functional cluster) was isolated. In this example cell extracts were prepared from INS-1 cells (BetaGene, Inc., Dallas Tex.) that had been stepped-down in low glucose and then stimulated with high glucose media for 2 hours as described above. Cell extracts were prepared by harvesting in RLB buffer as above. Following centrifugation, the cell extracts were brought to 300 mM NaCl and 15 mM EDTA (RLB-NaCl/EDTA). The extracts (500 ug protein) were incubated with 10 ug α-PTB (Zymed, Cat# 32-4800) or 10 ug of a control IgG (source, city, state) for 2 hours followed by a 1 hour incubation with 30 μl of protein A sepharose. The immunoprecipitates were washed 6 times in RLB-NaCl/EDTA. Optimization of immunoprecipitation of other RNA binding protein and associated components would be required. In examples of optimization, pH, ionic conditions, salt concentrations, reducing environment and incubation times can be varied.

RNA was extracted and purified from the immunoprecipitates using PicoPure RNA isolation kits (Arcturus). The purified RNA was quantified by RiboGreen (Molecular Probes) analysis and integrity of the samples was determined using a BioAnalyzer (Agilent). From these analyses approximately 25-30 ng of nucleic acid was associated with the control IgG immunoprecipitates. In contrast, approximately 200-900 ng of nucleic acid was immunoprecipitated by the PTB antibody. In order to obtain enough RNA for microarray studies, samples were subjected to two rounds of amplification using the MessageAmp kits and protocols (Ambion). Analysis of 10K Rat Pan Microarrays (MWG Ct#2250-000000) were performed as described for the RNA binding of protein arrays.

This analysis revealed a highly enriched (>5-fold) subset of approximately 450 genes. The normalized intensities of many of the genes were altered (>2-fold) in the clusters isolated from cells treated with 15 mM glucose whereas the same genes in the total RNA analysis were unchanged. This suggests that glucose could regulate the appearance of many mRNAs into or out of the cluster. Numerous predicted genes were highly enriched in the PTB-cluster and the presence of many of these was regulated by glucose. Included in this list are mRNAs for Glut2, glucokinase, phosphofructokinase, Kir6.2 (the ATP-sensitive K+-channel), SUR1 (sulfonylurea receptor 1), L-type Ca2+-channels, acyl-coa carboxylase and preproinsulin. In addition, and importantly, approximately 10% of the 450 genes in the PTB cluster had normalized intensity values at or below detectable levels when analyzed by microarray analysis of total mRNA samples. Thus, the ability to isolate the PTB cluster, purify and identify its associated mRNAs lead to the identification of very low abundant genes that most likely would have been missed or ignored in a normal array analysis. The ability to isolate the PTB cluster, enrich for a unique subset of genes, their regulated appearance in the cluster and identification of very low abundant genes supports the hypothesis regarding the role of RNA binding proteins in gene/protein expression and their utility for obtaining novel target and cellular pathway information. Expression of all candidate mRNAs in an RNP complex chosen for further downstream analysis are verified at the mRNA level by QRT-PCR using gene specific primers.

### Example 2

### Identification and Immunoprecipitation of Preproinsulin RNA Binding Proteins Using RIBOTRAP™

An alternative method for purifying and identifying RNA binding proteins is the RIBOTRAP™ assay (Ribonomics, Durham, N.C.). Two approaches for RIBOTRAP™ are described below. The first approach is an in vitro affinity-based assay using immobilized biotinylated oligonucleotides with sequences corresponding to RNA binding protein binding elements (Method 1). The second approach uses an affinity-tag placed on a full-length mRNA of interest or fragment of the mRNA of interest, which is expressed in a cell culture model and isolated using immobilized antibodies against the tag (Method 2).

To summarize Method 1, a cDNA representing a nucleic acid of interest or a portion of a nucleic acid that encodes an RNA binding protein binding site (e.g., a 5′ or 3′ UTR) is cloned using standard techniques into an expression vector possessing an appropriate mammalian cell promoter (e.g., a CMV, SV40, or actin promoter), or alternatively an adenovirus or retrovirus vector, and transfected into a compatible mammalian cell line. For the isolation of RNA binding proteins that participate in glucose and/or lipid metabolism, the cDNA may be expressed in a preadipocyte, adipocyte, or pancreatic beta cell line, for example. Following expression of the engineered cDNA, a cell extract is prepared that maintains the association between RNAs and their associated RNA binding proteins and mRNP complex-associated proteins, if present. The mRNA encoded by the transfected cDNA is affinity purified using an affinity protein that is known to bind to it, preferably one that does not interfere with the binding of the mRNA to its RNA binding protein(s). The affinity protein used may be linked to a solid matrix, such as agarose or Sepharose beads, and may be biotinylated or otherwise labeled (Method 1 below). Alternatively, the affinity protein may also be bound to the solid matrix indirectly via binding to an antibody that is bound to the solid matrix (Method 2 below). The affinity protein-matrix is used to isolate the expressed RNA, along with the RNA binding proteins and/or mRNP complex-associated proteins that are associated with the mRNA in vivo. Variations on the two methods include chemical crosslinking of the mRNP complexes with formaldehyde or the use of an epitope tagged or beaded binding element or an epitope tagged mRNA of interest.

Proteins that are isolated in association with the mRNA of interest using the RIBOTRAP™ assay are identified using standard proteomic methods. For example, Matrix Assisted Laser Desorption/Ionization—Time-of-Flight Mass Spectrometry (MALDI TOF) and Tandem Mass Spectrometry (or Mass Spectrometry/Mass Spectrometry (MS/MS)) are used to identify peptide sequences that can be subjected to database searches. Antibodies reactive with identified RNA binding proteins or mRNP complex-associated proteins are raised in mammals according to standard methods.

Methods and Materials

Method 1: In Vitro Affinity-Based Assay Using Immobilized Biotinylated Oligonucleotides

Probes for affinity-purification of preproinsulin RNA binding proteins were synthesized and biotinylated with biotin-modified T (thymidine) by art known methods (e.g., Ross et al. (1997) Mol. Cell. Biol. 17:2158-65). The probes for purification of preproinsulin RNA binding proteins were the following: a) for 3′-UTR element one 5′-gaauaaaaccuuugaaagagcacuac-3′, b) for 3′-UTR element two 5′-cccaccacuacccuguccaccccucugcaaug-3′, and c) for 5′-UTR element two 5′-agccctaagtgaccagctacagtcggaaaccatcagcaagcaggtcattgttccaac-3′. In addition, a negative control biotinylated probe (scrambled sequence) was used as described to identify and eliminate non-specific RNA binding proteins. The biotinylated probes were immobilized to streptavidin agarose (Pierce Biotechnology, Rockford, Ill.) or streptavidin magnetic beads (Dynal, Lake Success, N.Y.) overnight in a 1M NaCl-containing buffer as described (Ross et al., 1997). Beads were washed in high salt buffer to remove unbound probe, and then equilibrated in binding buffer. Cell extracts were prepared in RLB lysis buffer containing (50 mM HEPES, pH 7.5, 0.5% NP-40, 150 mM NaCl, 1 mM DTT, leupeptin 1 ug/ml, aprotinin 1 ug/ml and PMSF, 10% glycerol, 200 units/ml RNAse Out). The lysates are centrifuged at 10,000×g for 5 minutes and the supernatants (approx 1 mg/ml protein concentration) used in binding studies. Extracts were incubated with immobilized biotinylated probes (1-5 mg of coupled probe) for 4-12 hours at 4° C., washed, and proteins eluted in SDS-PAGE sample buffer. After separation by SDS-PAGE bands corresponding to proteins specifically bound to probes are identified by Western blotting or protein sequencing as previously described.

To specifically confirm binding of polypyrimidine tract binding protein (PTB) to the preproinsulin 3′ UTR, eluted PTB was analyzed by Western blot using commercially available PTB antibody (FIG. 7). Both recombinant PTB and native PTB derived from INS-1 cell lysates was evaluated for binding. FIG. 7 illustrates that PTB binds to the 3′UTR of preproinsulin but not the 5′UTR of preproinsulin.

FIG. 8 illustrates the current paradigm of glucose-regulated RNA binding protein binding of PTB (also referred to as RBP1) to the 3′ UTR of the preproinsulin mRNA, as well as putative binding of other unidentified PTB proteins. The 5′-UTR of preproinsulin mRNA contains a secondary (stem-loop) structure (ΔG=−10.8 kcal/mol) that is similar to structures found in other mRNAs that undergo regulation of biosynthesis at the translational level. Furthermore, the stem-loop structure is conserved in mammalian preproinsulin mRNAs. The 5′-UTR alone can function as a glucose and/or lipid response element. When both 5′- and 3′-UTRs are present, there is an even greater response to glucose. In addition, the glucose-stimulated translation is pancreatic beta cell-specific, since no glucose response is observed in non-beta cells. This strongly suggests the involvement of glucose and/or lipid regulated RNA binding proteins working via the 5′-UTR. Not to be limited to any particular theory, the data suggest a model in which at low or resting glucose levels, an RNA binding protein(s) is bound to the 5′-UTR of the preproinsulin mRNA and represses its translation. Increased nutrient concentrations (such as lipid and glucose) cause a change in the abundance or in the affinity of the RNA binding protein(s) for the preproinsulin 5′-UTR, thus relieving the repression and allowing enhanced translation of preproinsulin mRNA.

Method 2: Direct Affinity-Tagging of mRNA with an RNA-Epitope

A direct affinity-tagging of mRNA with an RNA-epitope assay is described below. This method is based on antibody-recognition of a unique RNA stem loop structure. The well-characterized antibody α-g10 (i.e., α-T7-tag) is raised against the N-terminus of a g10 fusion protein by standard methods. This antibody is used to screen a complex library of degenerate RNAs (10⁶ molecules) representing various stem loop structures. Following stringent washing conditions, a single 40 nucleotide RNA species is identified (D10) that was specifically recognized by α-g10. Upon further characterization, the D10 RNA is shown to mimic the peptide antigen; thus one can use the peptide for competition or elution. When the RNA-epitope is inserted into an mRNA, the RNA epitope-tagged mRNA can be specifically recovered from a mixture of total cellular mRNAs using α-g10. Furthermore, the antibody alone has no reactivity with total eukaryotic cellular mRNAs.

The D10 RNA-epitope tag is placed at the end of the 3′-UTR of the gene for Nkx6.1 and preproinsulin by methods well-known to the skilled artisan. This is accomplished by PCR cloning the tag into the full-length cDNAs for Nkx6.1 or preproinsulin (obtained by PCR cloning). These constructs are used for 1) generating in vitro transcripts for competition and affinity reagents, and 2) overexpression of Nkx6.1 or preproinsulin in a mammalian cell culture model followed by recovery of the RNA epitope-tagged mRNA from cell extracts with α-g10.

For the preproinsulin studies, the D10 RNA epitope-tagged preproinsulin cDNA as subcloned into pcDNA3.1 neo and used to transfect MIN-6, α-TC1.6, and NIH3T3 cells. Transiently transfected cells as well as established stable transfectants (selected with Neo) are examined. Once expression of the tagged mRNA is confirmed by RT-PCR, extracts are prepared as described above from cells incubated in low or high glucose. Mock transfected cells are also examined.

Construction and transfection into the various cell-types of a D10 RNA epitope-tagged Nkx6.1 is performed in a similar manner. For analysis, the RNA epitope-tagged mRNAs are isolated from the extracts using immobilized α-g10. Proteins in these complexes are eluted with SDS-PAGE sample buffer or using antigenic peptide (NH₂-MASMTGGQQMGRC—COOH), which was previously shown to compete for the D10 epitope. A comparison of protein profiles obtained from the various cell extracts (including mock transfected cells) identifies unique protein bands. The eluted proteins are processed as described in Example 1 above to obtain peptide sequence. One variation on this procedure included D10-tagging of a fragment of the full-length mRNA (e.g., the 5′- or 3′-UTR alone containing the D10 epitope).

A comparison of RNA binding protein expression profiles from α-TC1.6 cells, pancreatic beta cells (which express both homeodomain transcription factor Nkx6.1 mRNA and protein), and NIH3T3 cells is performed to identify cell-type specific RNA binding proteins using RIBOMAP™. These RNA binding proteins represented candidate proteins that control Nkx6.1 expression.

RAS™ is then performed using antibodies to these candidate RNA binding proteins and the resulting functional clusters analyzed for Nkx6.1 mRNA expression. A functional cluster containing Nkx6.1 mRNA could contain other mRNAs that are coordinately regulated, and may code for proteins involved in development of the endocrine pancreas and/or pancreatic beta cell differentiation. Proteins that bind to the 5′-UTR of Nkx6.1 mRNA are also purified.

Specificity and Mapping of RNA Binding Protein Binding Elements

In order to verify potential RNA binding proteins and their binding specificity, competition experiments using immobilized binding sites (either biotinylated probes or D10 epitope-tagged probes generated by in vitro transcription) are performed. For example, the specific binding site is immobilized with either streptavidin agarose or α-g10 agarose and incubated with cell extracts or a recombinant RNA binding protein according to art known methods. The binding reactions are carried out in the absence or presence of increasing concentrations of control or competing non-biotinylated or non-tagged probes (synthetic oligonucleotides or oligonucleotides generated by in vitro transcription, as described above). Binding is analyzed by 1) electrophoretic mobility shift assays as described in the art and/or 2) SDS-PAGE followed by Coomassie staining, to detect the presence or absence of RNA binding protein bands. RAS™ may also be performed as a third verification procedure. In this case antibodies raised against the RNA binding protein are used to immunoprecipitate complexes as described above and microarray analysis is performed to identify the associated mRNAs, one of which should be the original endogenous target mRNA.

### Example 3

### Analysis of RNA Binding Protein Expression and Associated mRNAs in Human Adipocytes and Preadipocytes

Adipocytes have long been considered a primary location for glucose disposal and energy storage in the form of triglycerides (fat). Adipocytes also comprise critical endocrine tissue that not only responds to insulin through glucose uptake and lipogenesis, but also synthesizes and secretes a variety of signaling molecules involved in systemic energy homeostasis. An analysis of RNA binding proteins and their associated mRNAs and mRNP complex-associated proteins and their role in gene expression in adipocytes provides a better understanding of adipocyte function and can identify targets for therapeutics that treat conditions associated with aberrant glucose or lipid metabolism. A flow chart for an exemplary adipocyte analysis is provided in FIG. 9.

RNA binding proteins that are enriched in mature adipocytes vs. preadipocytes in lean individuals (BMI<24) were identified as follows. Briefly, human preadipocytes were harvested from elective liposuction from three lean individuals according to standard procedures. A portion of the preadipocytes were differentiated in culture to mature adipocytes (Zen-Bio, Durham, N.C.). The expression pattern of RNA binding proteins in mature adipocytes was compared to the expression pattern of RNA binding proteins in preadipocytes using a RIBOCHIP™ V.1 array (MWG Biotech, High Point, N.C.) according to the methods described in Example 1. FIG. 10 provides a list of the RNA binding proteins and corresponding genes that are differentially regulated in adipocytes vs. preadipocytes. In another experiment, the RNA binding protein expression in preadipocytes from obese individuals was compared to expression in mature adipocytes in obese individuals. Preadipocytes and adipocytes were obtained from obese individuals as described above. RNA binding proteins were identified using RIBOCHIP™ analysis as described in Example 1. FIG. 11 provides a list of 14 RNA binding proteins and their corresponding genes that were induced 2 fold or more in mature adipocytes from obese individuals as compared to preadipocytes from obese individuals.

The effects of insulin or the beta 3 agonist, BRL-37344, on RNA binding protein expression in human mature adipocytes was also examined. Mature adipocytes from lean individuals were obtained as described above and either left untreated (basal) or treated with 100 nm insulin or 1 μM BRL-37344 and RNA prepared from these cells (Zen-Bio, Durham, N.C.). Differential expression of RNA binding proteins were identified using RIBOCHIP™ analysis as described above. FIG. 12 provides a list of the RNA binding proteins and corresponding genes that are differentially regulated in response to treatment with BRC-37344. FIG. 13 provides a list of the RNA binding proteins and corresponding genes that are differentially regulated in response to insulin.

In addition, the expression pattern of RNA binding proteins in mature adipocytes from three lean individuals was compared to the expression pattern of RNA binding proteins in mature adipocytes from three obese individuals (BMI>30). Preadipocytes were obtained by elective liposuction and cultured as described above. Adipocytes from obese individuals showed an altered pattern of RNA binding protein expression.

These data provide a refined list of candidate RNA binding proteins for further validation for participation in an adipocyte pathway, insulin production or insulin action, insulin resistance, a lipogenesis pathway, diabetes, obesity, and/or glucose and lipid metabolism pathway, or any pathway that participates in an aspect of glucose and lipid metabolism, and for the isolation of associated mRNP complex-associated proteins, and associated RNAs.

### Example 4

### Analysis of RNA Binding Protein Expression in Rat Pancreatic Beta Cells Treated with Glucose

The effect of glucose on RNA binding protein expression in rat pancreatic beta cells was examined. A derivative of the INS-1 rat pancreatic beta cell line, clone 832/13, was chosen because of its ability to mimic many of the normal functions of beta cells of pancreatic islets. Whereas INS-1 cells respond to glucose treatment with a 2-4 fold increase in insulin secretion, clone 832/13 is induced 8-13 fold by glucose treatment.

Briefly, 832/13 cells were grown RPMI containing 10% fetal bovin serum (Invitrogen, Corp., Carlbad, Calif.) to near confluence, shifted to low glucose (3 mM) for 1 hour, and treated for 2 hours with fresh medium containing 3 mM or 15 mM glucose. RNA was prepared and differential gene expression of the RNA binding proteins was determined using the RIBOCHIP™ as described abvove. FIG. 14 provides a list of RNA binding proteins and their corresponding genes that displayed a 2-fold up- or down-regulation as a result of glucose treatment.

These data provide a refined list of candidate RNA binding proteins for further validation for participation in an adipocyte pathway, insulin production or insulin action, insulin resistance, a lipogenesis pathway, diabetes, obesity, and/or glucose and lipid metabolism pathway, or any pathway that participates in an aspect of glucose and lipid metabolism, and for the isolation of associated mRNP complex-associated proteins, and associated RNAs.

### Example 5

### Identification of Differentially Expressed RNA Binding Proteins in HepG2 Cells in Response to Peroxisome Proliferator Activated Receptor Ligands

The effects of peroxisome proliferator activated receptor (PPAR) ligands on human RNA binding protein expression was examined in the human hepatocyte cell line HepG2. Liver is a major insulin target tissue and one of the PPAR receptors, PPARγ, is thought to be the major biological target for a number of insulin sensitizing agents, including thiazolidinediones, L-tyrosine derivatives, halogenated fatty acids and prostaglandins. The compounds profiled include prostaglandin J2, perfluorooctanoic acid, 2-bromohexadecanoic acid, Ciglitazone, Troglitazone, GW-9662, MCC-555, Wyeth 14643, and Bezafibrate. Profiling the effects of these compounds using the RIBOCHIP™ was expected to reveal changes in regulatory genes important for the pharmacological and toxicological properties associated with these agents. Common themes or patterns in gene expression likely represent common pharmacology and toxicology while distinct gene expression changes elicited by individual compounds or subsets of compounds likely represent unique pharmacological or toxicological properties. The changes in gene expression identified in this manner are therefore attractive candidates for validation surrounding participation in the mechanism of insulin action and the pharmacological and toxicological properties of PPARγ ligands.

Briefly, HepG2 cells (obtained from ATCC (www.atcc.org; catalog number HB-8065)) were maintained as recommended in Minimal Essential Medium (MEM) with 10% fetal bovine serum (FBS) supplemented with antibiotics in p150 plates at 37° C., 5% CO₂. Cells were split 1:5 and fresh media added every 3 days. Cytotoxicity was assessed using the Alamar Blue-based CellTiter™ Blue Cell Viability Assay (Promega; Madison Wis.) to determine the viable cell fraction that remained following a 72 hour period. Cells (˜8,000 cells/well) were plated in 96 well BioCoat collagen coated plates (Becton Dickinson; Bedford, Mass.) using standard media. This allowed untreated control samples (0.25% DMSO) to be in late log phase (˜70% confluent) at completion of the study. Cells were then allowed to recover for 24 hours at 37. C, 5% CO₂. A two (2) fold dilution series was prepared for each compound starting at 3.0 mM in MEM containing 0.1% BSA (instead of 10% FBS) but without phenol red or antibiotics. Following the cell recovery period, the media was removed and fresh media containing compound was added. Treatments were performed in triplicate for each compound at each dose. Cells were incubated with compound for 72 hours at 37° C., 5% CO₂. The viable cell fraction remaining was determined by washing the wells with fresh media without indicator, lysis of the remaining live cells by addition of 0.9% Triton X-100 in water, and performing the Alamar Blue assay as described in the CellTiter™ Blue Cell Viability Assay product literature. The concentration resulting in 50% cell death relative to a vehicle only control following 72 hours of treatment (LD₅₀) was determined using Prism 4.0 (GraphPad; San Diego, Calif.) dose-response analysis.

RNA for microarray analysis was obtained from cells treated for 24 hours at the determined LD₅₀. Typically, ˜1.5×10⁶ cells were plated in a p100 dish and allowed to settle for 24 hours by incubation at 37° C., 5% CO₂ in MEM+10% FBS without antibiotics. Old media was removed and fresh MEM+0.1% BSA without antibiotics containing compound at LD₅₀ concentration and 0.25% DMSO was added to the flask. A vehicle only treatment was also performed. Duplicate treatments were performed for each compound as well as for vehicle only controls. The cells were incubated with compound for 24 hours at 37° C., 5% CO₂ following which they were harvested by scraping (without trypsinisation) and centrifugation. The cells pellets were flash frozen and stored at −80° C. until ready for RNA extraction.

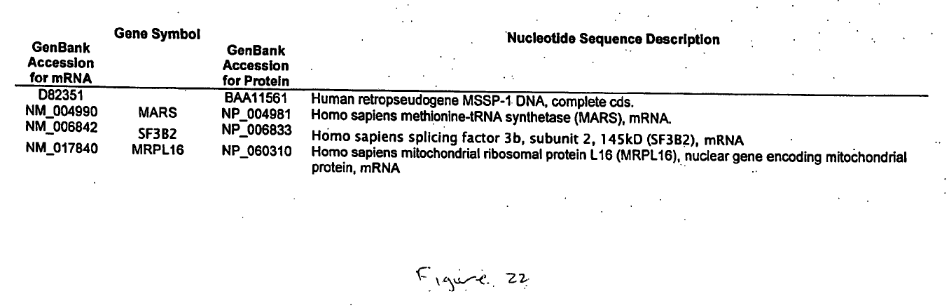

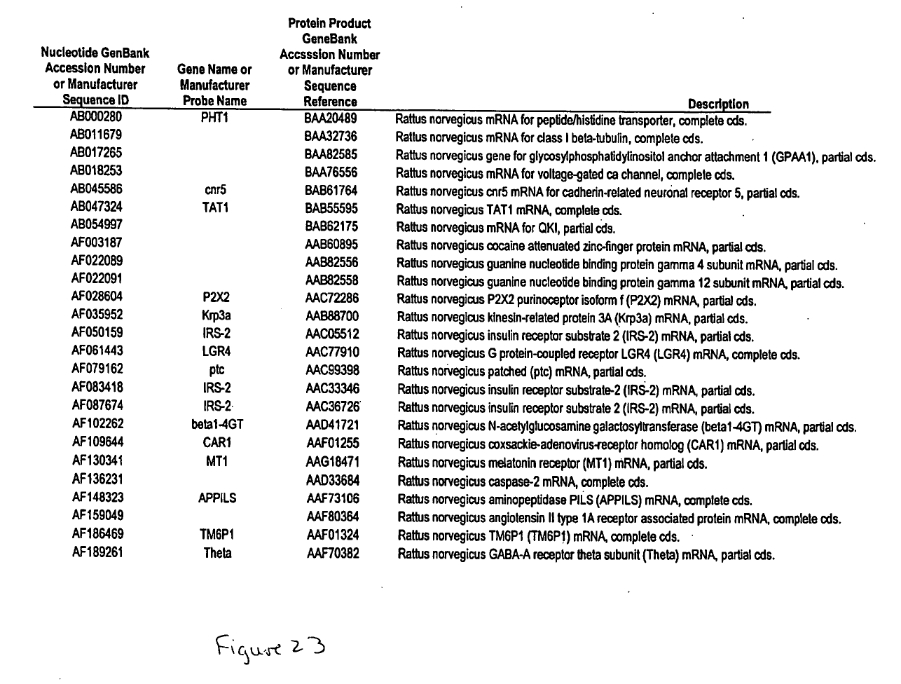

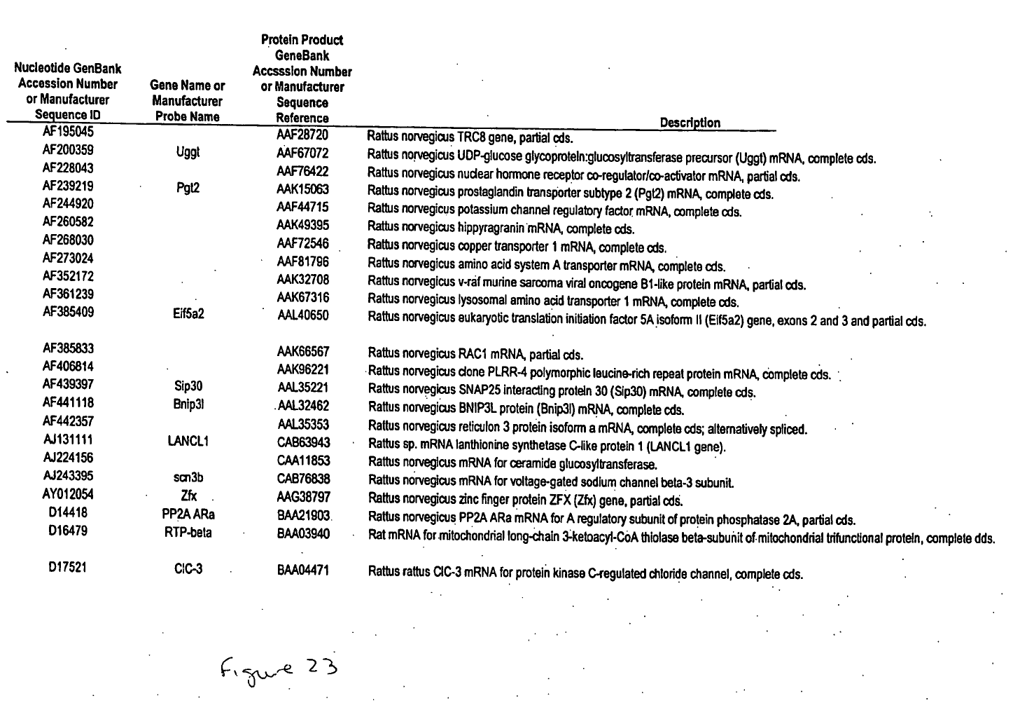

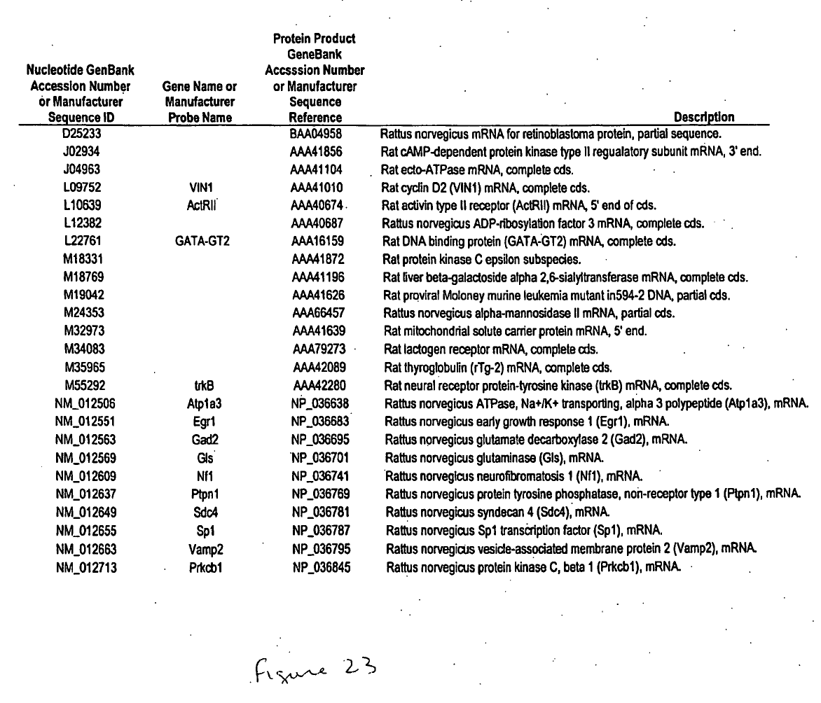

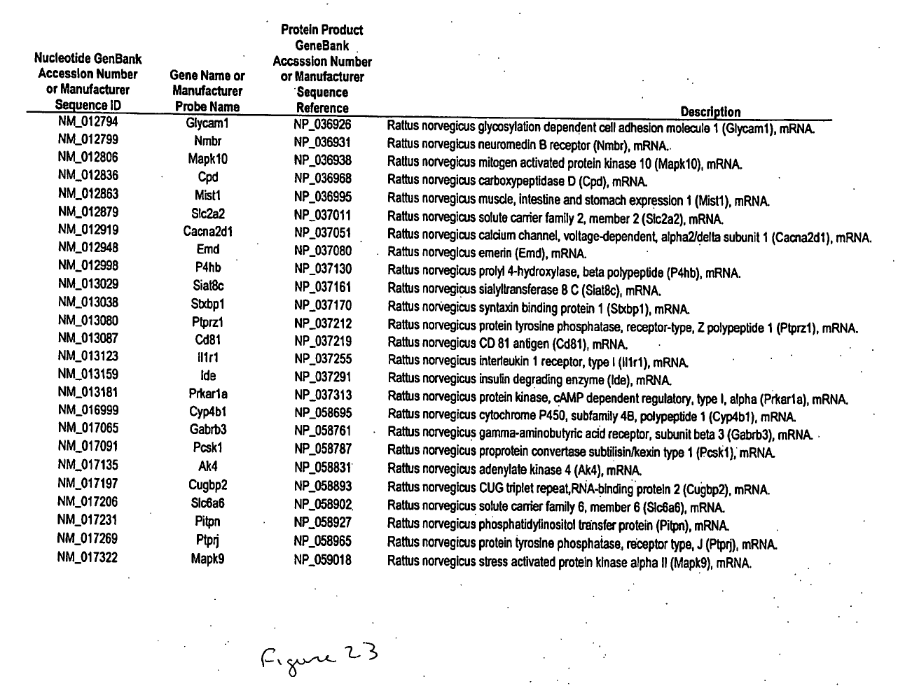

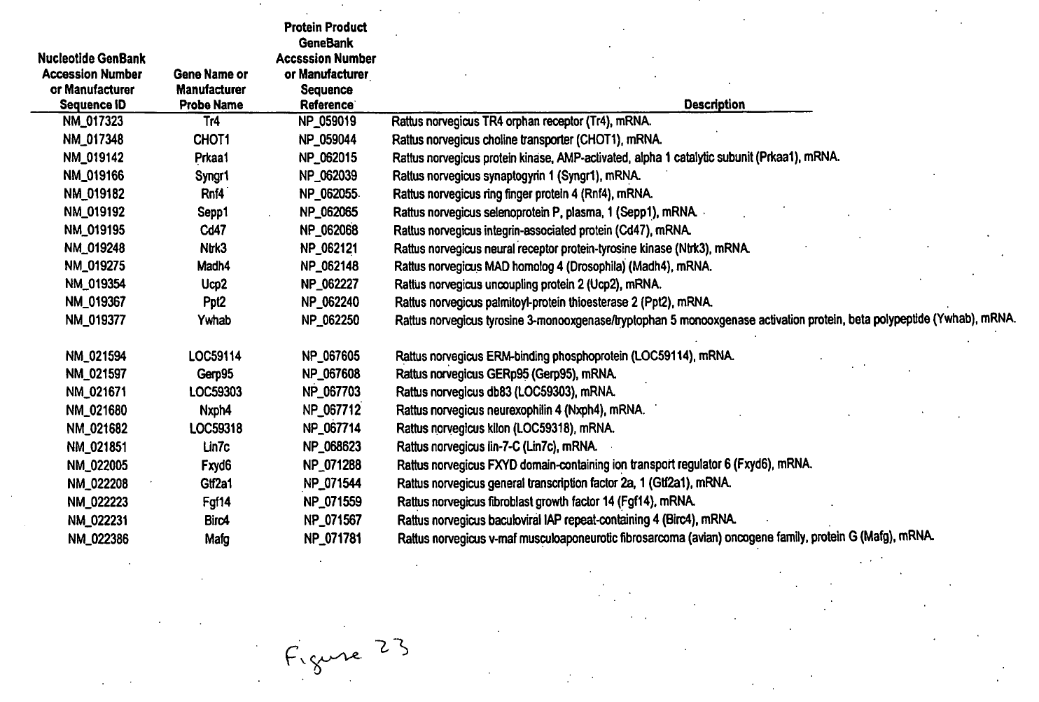

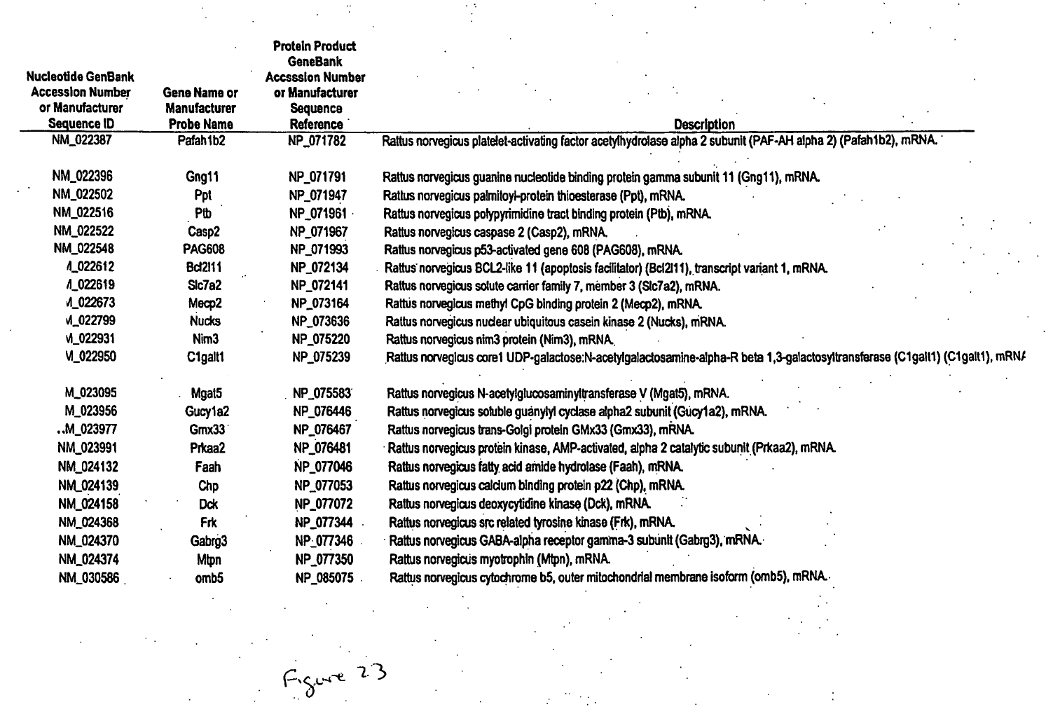

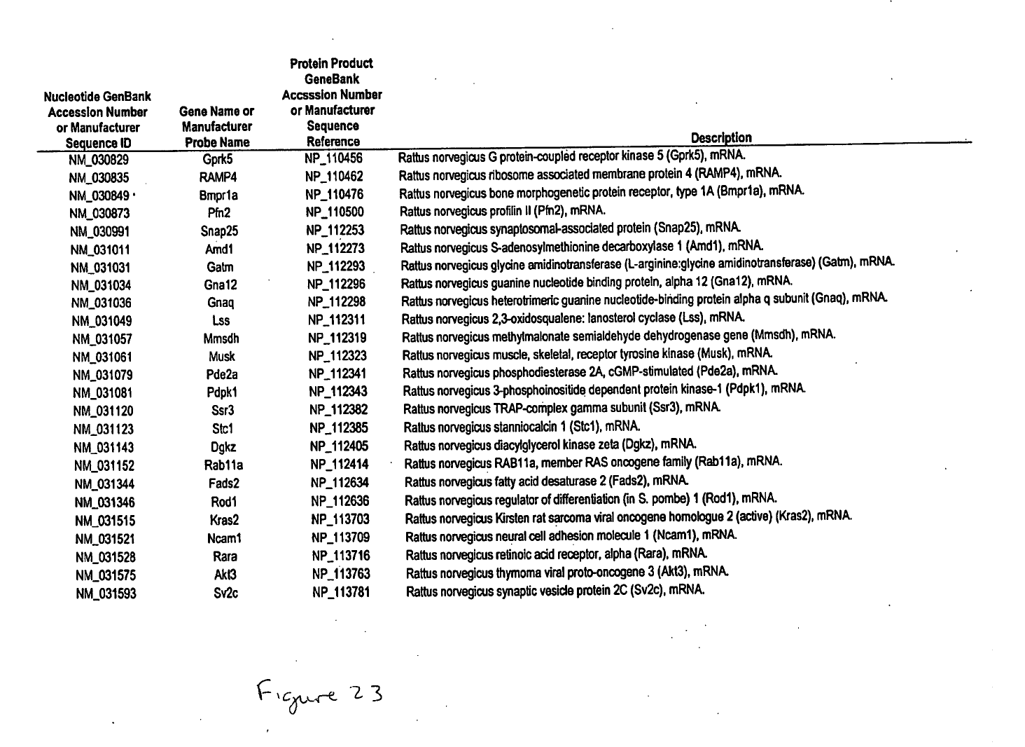

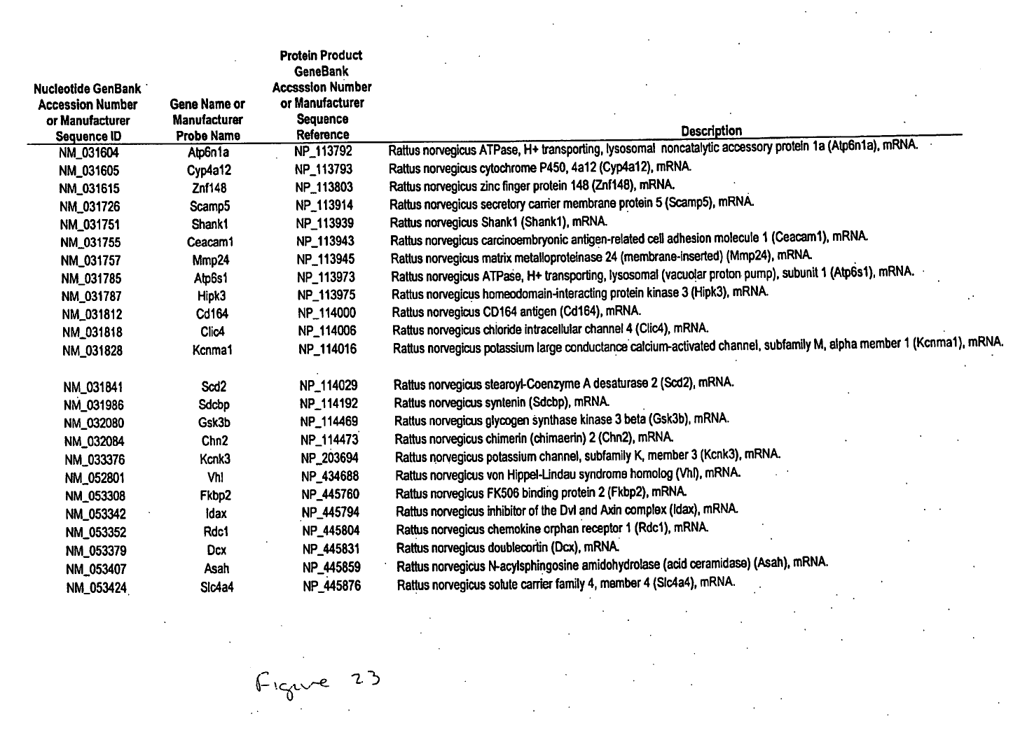

Total RNA was extracted and analyzed for using the RIBOCHIP™ as described in Example 1. ANOVA analysis (p-value≦0.05) was used to identify genes that were differentially expressed for each treatment compared to a vehicle only control (0.25% DMSO). FIGS. 15-22 provide lists of RNA binding proteins and their corresponding genes that are differentially expressed in HepG2 cells treated with bezafibrate (FIG. 15), Wyeth 14642 (FIG. 16), troglitazone (FIG. 17), MCC-555 (FIG. 18), ciglitazone (FIG. 19), 2-bromohexadecanoic acid (2-BHDA) (FIG. 20), prostaglandin J2 (PJ2) (FIG. 21), and perfluorooctanoic acid (PFOA) (FIG. 22).

### Example 6

### In Vitro RAS™ Identification of mRNAs Associated with Polypyrimidine Tract Binding Protein Complexes Using the Purified Recombinant RNA Binding Protein

As and alternate approach to in vivo RAS™ performed using antibodies against the endogenous RNA binding protein or epitope-tagged RNA binding proteins, an in vitro RAS™ was used. In brief, cytoplasmic extracts from cells or tissues or purified RNA from cell or tissues is incubated with a purified recombinant RNA binding protein immobilized on a solid support. The example given below is an in vitro RAS™ assay performed using GST-PTB and purified RNA or cytoplasmic extracts prepared from INS-1 cells.

Cloning and Expression of RNA Binding Protein Genes that Regulate Insulin

The human PTB cDNA was cloned into a pGEX4T vector, which contains a GST affinity tag, and expressed in E. coli cells. The GST-PTB fusion protein was purified from bacterial lysates using the GST affinity tag, as described above.

Isolation of RNAs that Bind to PTB In Vitro

INS-1 cells were cultured as described in Example 2. Cells were placed on ice, washed 3 times with ice cold PBS and lysed in 1 ml/dish of lysis buffer (50 mM Hepes, pH 7.2, 0.5% NP40, 150 mM NaCl, 2 mM MgCl₂, 5% glycerol, 1 mM DTT, 10 ug/ml Aprotinin, 1 ug/ml Leupeptin, 0.2 mg/ml PMSF and 200 U/ml RNAseOUT (Invitrogen, Carlsbad, Calif. Cat# 10777-019). Cytosolic fractions were isolated by centrifuging the lysates at 3700 g for 10 minutes at 4° C. The supernatant was transferred to a fresh tube and the NaCl concentration was raised to 300 mM and EDTA added for a final concentration of 20 mM. This sample was then centrifuged at 10000 g for 10 minutes at 4° C. The supernatant is considered the cytoplasmic extract containing mRNA. As an additional sample, RNA is also purified from these extracts using Qiagen kits as previously described.

The GST-PTB fusion protein was used to screen for mRNAs that bind to PTB. Briefly, the purified GST-PTB fusion protein was bound to a glutathione sepharose (Amersham, Uppsala, Sweden. Cat# 17-0756-01) support through the GST linkage according to standard methods.

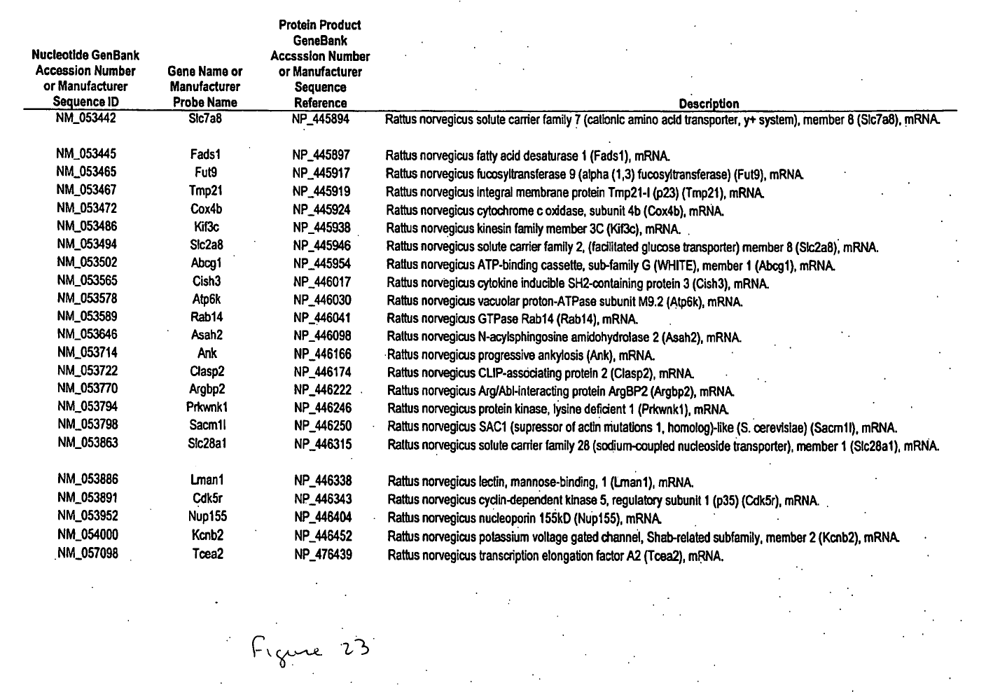

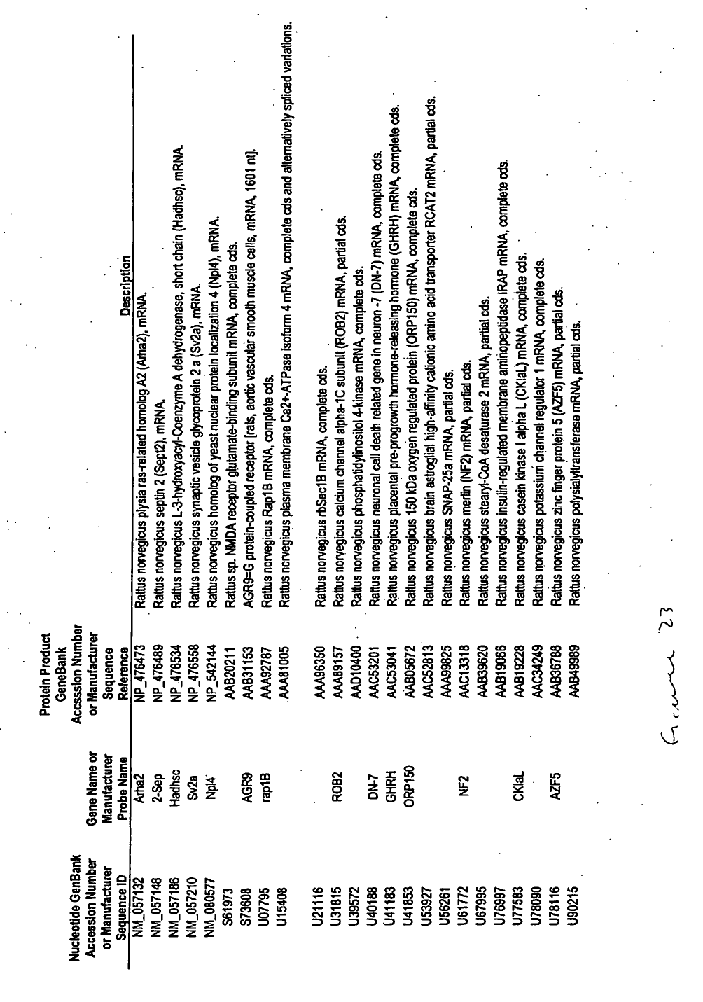

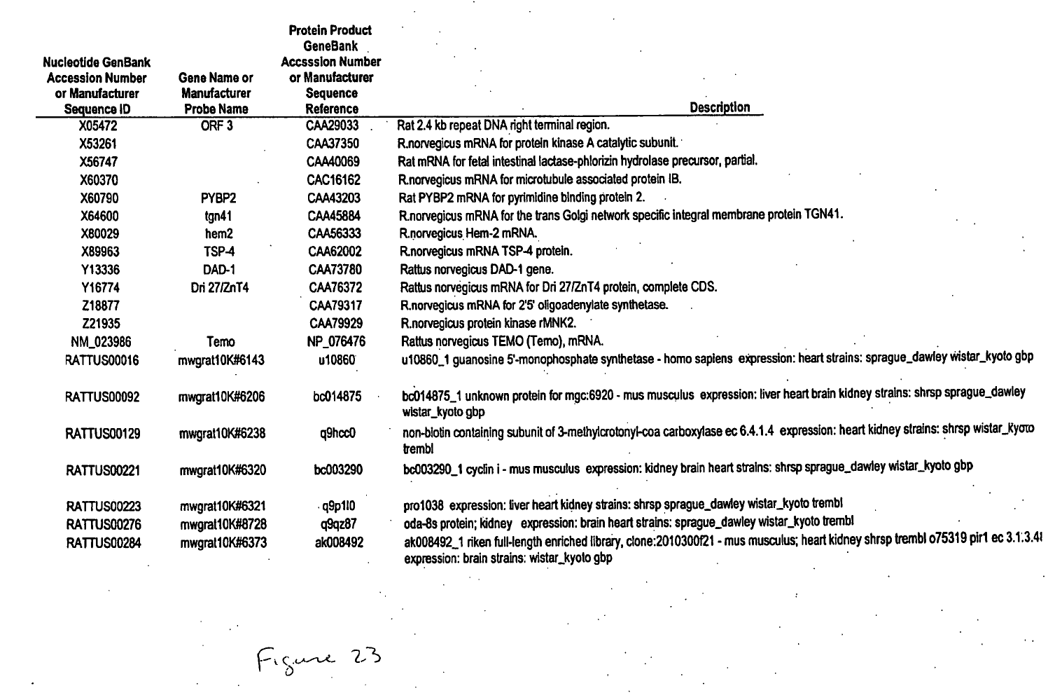

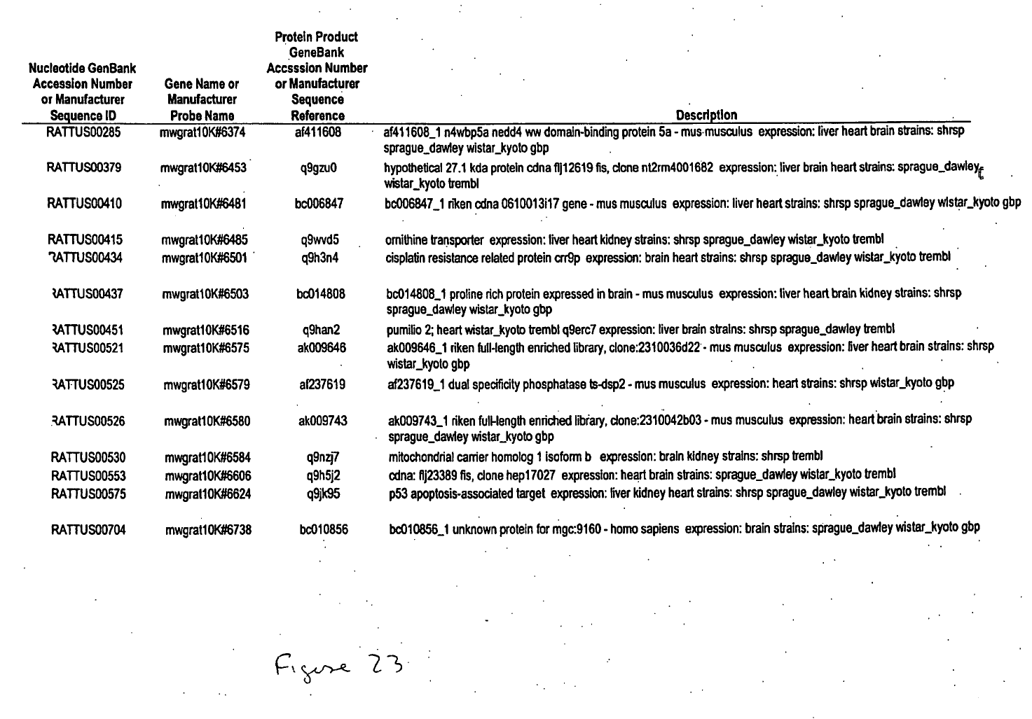

Purified RNA or cytoplasmic lysates containing mRNA were incubated with the bead-bound GST-PTB fusion protein for 2 hours at 4° C. RNAs that bind to GST-PTB were retained on the beads. Ionic conditions for binding and washing were altered to select for high affinity binding of mRNAs to PTB or other RNA binding proteins, as described above. In this case, beads were washed 5 times with binding buffer (50 mM Hepes, pH 7.2, 0.5% NP40, 300 mM NaCl, 20 mM EDTA, 2 mM MgCl₂, 5% glycerol, 1 mM DTT, 10 ug/ml Aprotinin, 1 ug/ml Leupeptin and 0.2 mg/ml PMSF). After the final wash, the beads were resuspended in 350 ul of RNAeasy mini prep buffer RLT and purified RNA using RNAeasy mini prep protocol (Qiagen, Valencia, Calif. Cat# 74104). Alternatively, bound mRNAs are selectively eluted with 10 mM glutathione (Sigma, St. Louis, Mo.), according to standard methods, which competes with GST to displace the mRNA-RNA binding protein complexes from the beads. Glutathione elution enables the selective elution of only those mRNAs that are bound to the RNA binding protein, and minimizes contamination with mRNAs that are non-specifically associated with the sepharose matrix. As a positive control, eluted mRNAs were enriched for the presence of preproinsulin mRNA, which was directly assessed using QRT-PCR, according to standard methods. The eluted and purified RNAs are then identified by microarray analysis as described in Example 1. FIG. 23 provides a list of genes bound to purified recombinant GST-PTB.

RAS™ Performed with an Epitope-Tagged RNA Binding Protein Expressed in Cells or Tissues

As an alternative approach to in vivo RAS™ using antibodies against the endogenous RNA binding protein or to in vitro RAS™, epitope-tagged versions of RNA binding proteins are expressed in a cell or tissue of interest. For example, a T7-epitope tagged PTB (T7-PTB) is transfected and expressed in INS-1 cells. The addition of the epitope tags streamlines the ability to immunoprecipitate the RNP complexes from the cells, since under most circumstances the epitope is not buried within the complex. Following stable selection of T7-PTB, mRNP complexes containing the T7-PTB are isolated from cell extracts using RLB buffer as described and the T7 monoclonal antibody (Novagen, Madison, Wis.). RNA is extracted and identified by microarray analysis as described.

The combined in vitro and in vivo analysis of RNP complexes offers a powerful approach to the study of post-transcriptional regulation. The comparative analysis identifies the set of genes being coordinately regulated in a variety of approaches. For the genes associate with PTB in INS-1 cells, these data provide a roadmap of the regulatory, metabolic, and signaling pathways that act in concert to orchestrate the proper production and secretion of insulin, for example. Analysis of dynamic changes in the PTB mRNP complex has lead to the identification of novel diagnostic biomarkers and a collection of compelling therapeutic targets for modulating insulin production or other gene involved in glucose and/or lipid metabolism, insulin action, insulin resistance, diabetes and obesity.

### Example 7

### Validation of Potential Therapeutic Targets and Components of Cellular Pathways by RNAi-Mediated Silencing of Genes

Once genes within a ribonomic cluster are identified, in order to validate them as a potential therapeutic target or to place them in cellular pathways, RNAi-mediated gene silencing was performed to verify their importance in the mRNP complex. SMARTPOOL™ designed siRNAs (Dharmacon (Lafayette, Colo.) were used, which contain a mixture of siRNAs that specifically targeted a gene of interest, resulting in a greater than ≧50% reduction in the target mRNA within 24 h post-transfection.

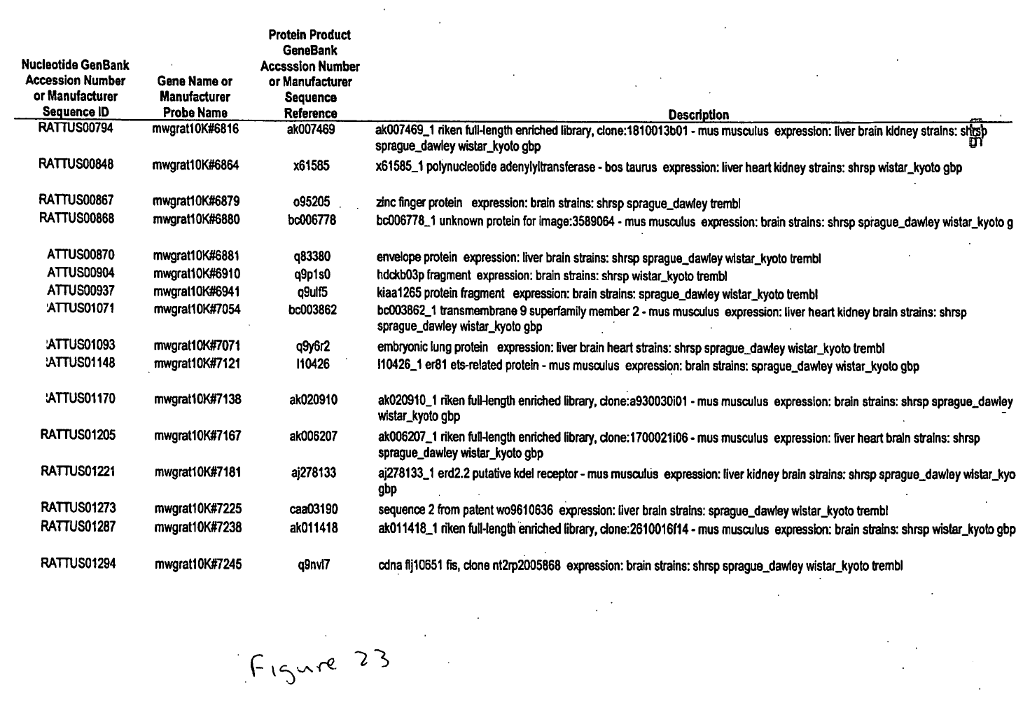

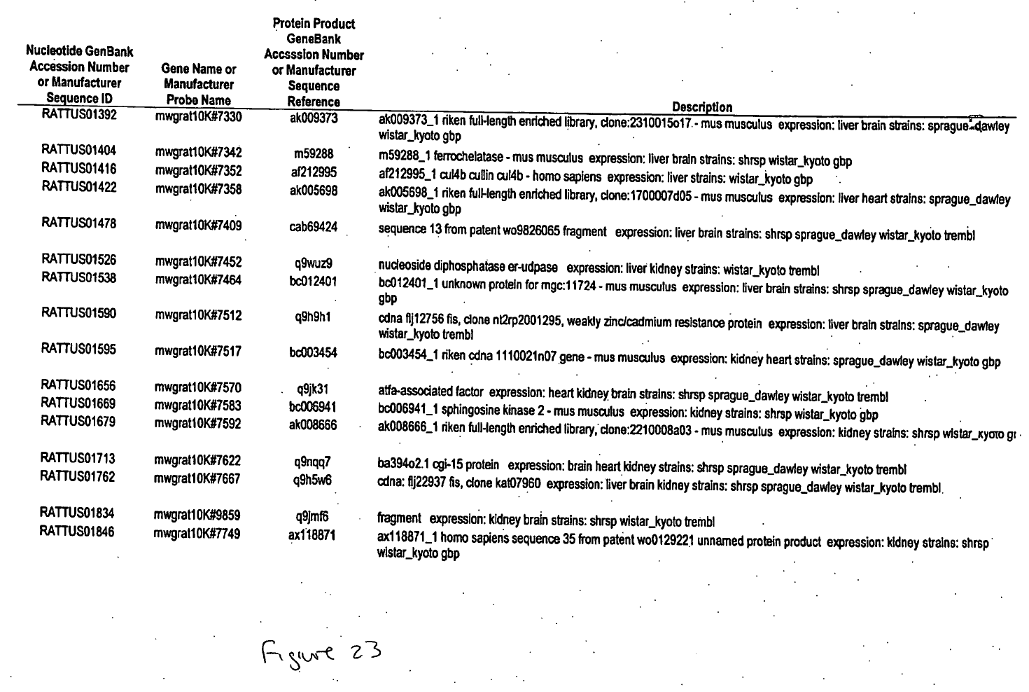

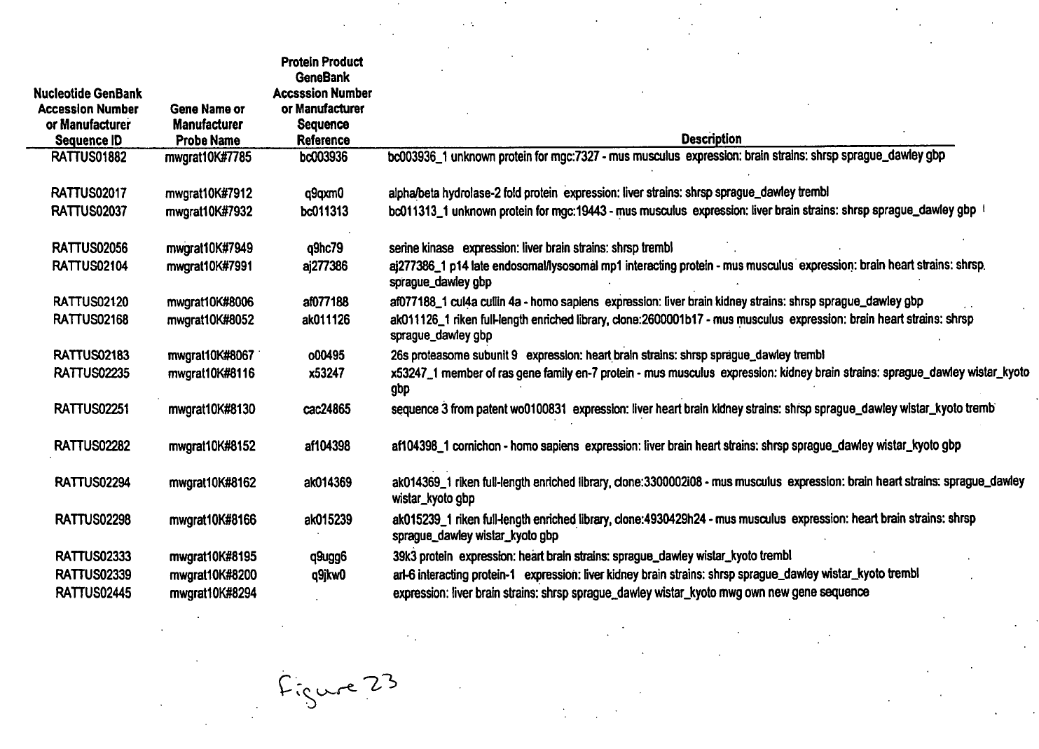

SMARTPOOL™ siRNAs the ion channel nucleic acids that had previously not been associated with glucose-stimulated insulin secretion, included CNCG (cat# M-003833-00-05), CaCNA2D1, KCNC3 (cat#M-003838-00-05), and KCNB2 (cat#M-003830-00-05). Transfection of each siRNA was performed in INS-1 cells that were plated in 24-well culture dishes, and incubated with fresh RPMI media containing 10% fetal bovine serum 90 minutes prior to transfection. Transit TKO transfection reagent (Dharmacon, Lafayette, Colo.), 2 μl, was incubated for 15 minute at room temperature with SMARTPOOL™ siRNAs at a concentration range to yield a final concentration of 1-50 nM siRNA on the cells. After a 24 hour incubation at 37° C., the cells were processed for total RNA isolation and glucose-stimulated insulin secretion. Expression of target genes in untreated, control transfected and sequence-specific siRNA-transfected cells was assessed by QRT-PCR and/or immunoblotting. For insulin secretion, cells were incubated for 60 minutes in serum-free media containing 3 mM glucose. The media was then changed to fresh media containing either 3 mM glucose or 15 mM glucose and incubated for 120 minutes. Conditioned media from each sample was then used to determine the levels of secreted insulin using an insulin ELISA (Linco Research Products, St. Charles, Mo. Cat#EZHI-14K). Compared to cells transfected with the control siRNA, transfection of INS-1 cells with siRNA to PTB (FIG. 24A), CNCG (FIG. 24B), KCNC3 (FIG. 24B), KCNB2 (FIG. 24B) and CaCNA2D1 (FIG. 24C) showed altered insulin secretion suggesting that these are involved in the insulin secretory pathway (FIG. 19). In addition, extensive time course experiments, glucose dose response experiments, and experiments that determine the ability to respond to other secretagogues, such as sulfonylureas, GLP-1 and fatty acids, can be performed.

RNAi-mediated gene silencing of the two potassium channels KCN3 and KCNB2 caused an extreme increase in basal insulin secretion levels, suggesting these channels play a functional role in the process. These two potassium channel proteins were not previously implicated in regulating insulin secretion or pancreatic beta cell function. This is significant, since the action of a class of diabetes drugs (sulfonyureas or gliburides like GLUCOVANCE) act by inhibiting a K⁺ channel on the pancreatic beta cell. This inhibition leads to membrane depolarization, which allows calcium to enter the cell and stimulate release of intracellular secretory granules filled with insulin. These drugs act by increasing overall and basal insulin secretion, thereby controlling high glucose levels (hyperglycemia). These results suggest that there are additional K⁺ channels that may work in this process and provide candidate targets for new diabetes drugs.

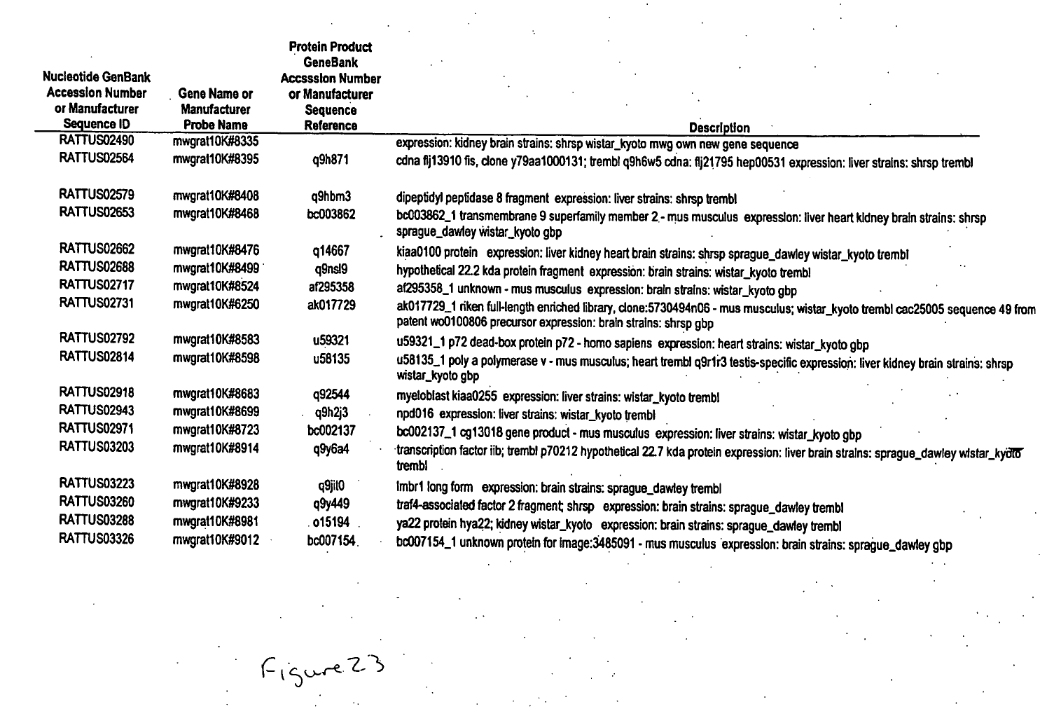

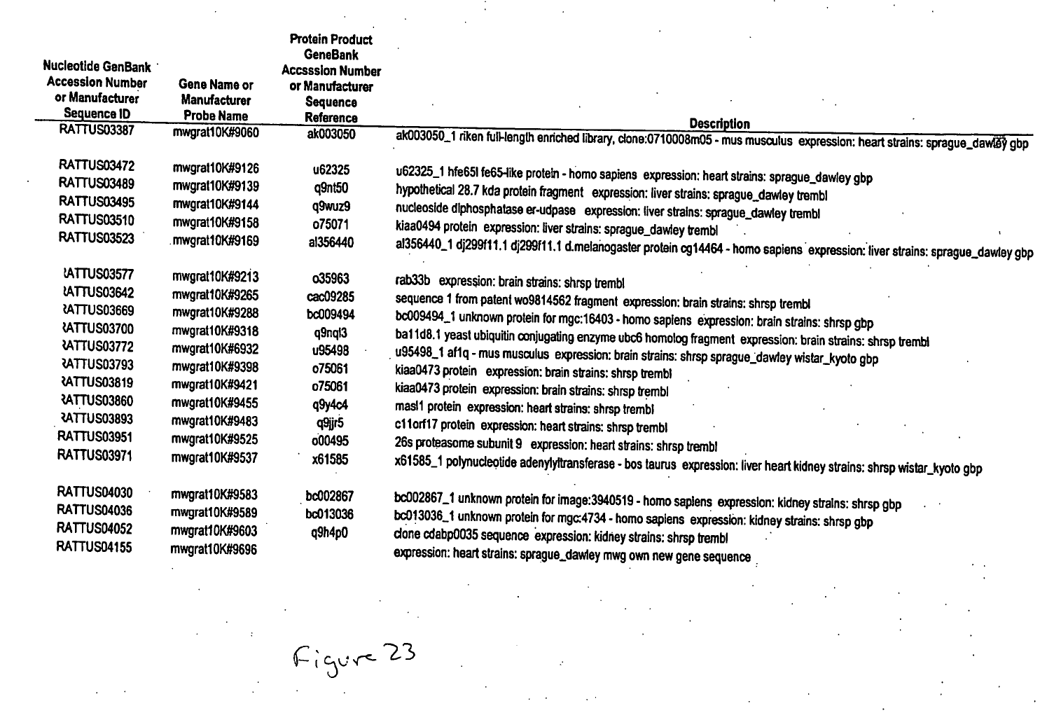

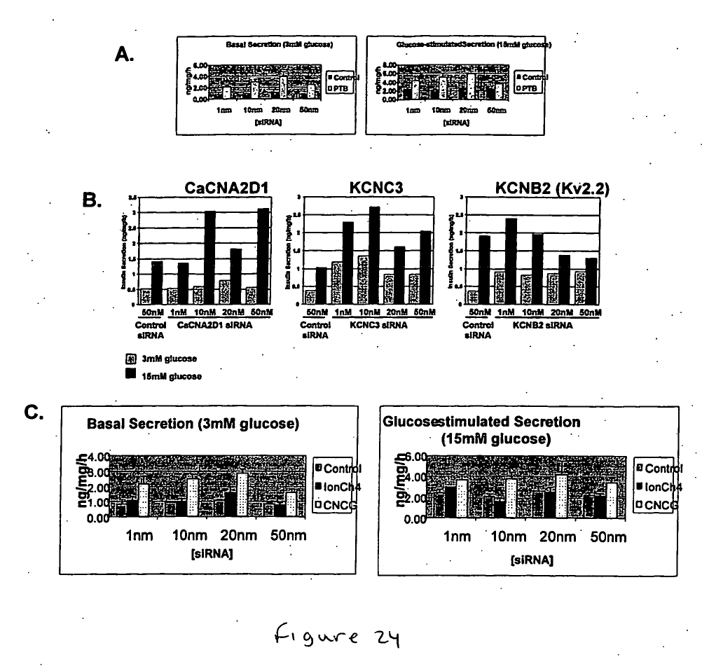

It is notable that many of the ion channel proteins identified on the PTB cluster were not previously identified as participating in glucose and lipid metabolism. These proteins represent targets for new therapeutics that may be used to regulate a pathway that participates in glucose and lipid metabolism or other pancreatic beta cell function. FIG. 25 illustrates some of the known pathways that participate in insulin secretion in pancreatic beta cells, indicating some of the proteins encoded by mRNAs found on the PTB cluster.

Over-Expression of Target Proteins

Alternatively, cells can be transfected with nucleic acids encoding target proteins or treated with a transcriptional enhancer for a gene encoding a target protein of interest, in order to overexpress a particular target protein identified by the methods of the invention. These systems would then be subject to biological assays (e.g., glucose-stimulated insulin secretion) as described above.

### Example 8

### RIBOTRAP™ Characterization of PTB on the 3′-UTR of Preproinsulin mRNA

RIBOTRAP™ experiments were performed in order to characterize the effect of glucose on the binding of PTB to the 3′UTR of preproinsulin.

Preparation of Cell Extracts: INS-1 cells were incubated in RPMI media containing 0.5 mM glucose for 2 hours. The cells were washed and the medium replaced with RPMI containing either 0.5 mM (low glucose) or 15 mM (high glucose) for various times up to 2 hours. The cells were washed with cold PBS and harvested in 1 mL RLB lysis buffer (50 mM HEPES, pH 7.5, 0.5% NP-40, 150 mM NaCl, 1 mM DTT, leupeptin 1 μg/ml, aprotinin 1 μg/ml and PMSF, 10% glycerol, 200 units/ml RNAse Out). The lysates were centrifuged at 10,000×g for 5 minutes and the supernatants (approx. 1 mg/ml protein concentration) were used in binding studies.

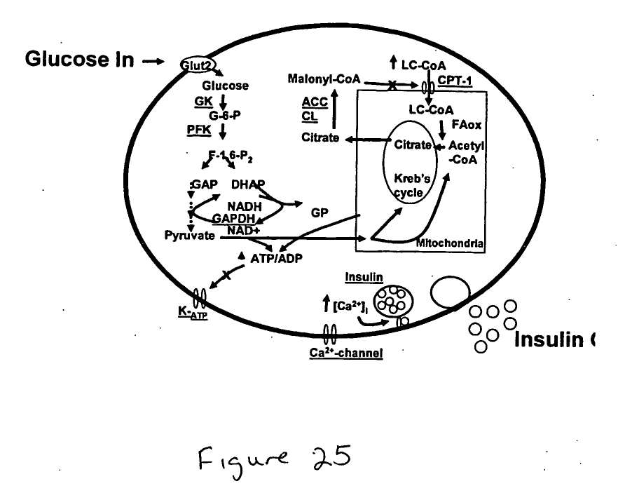

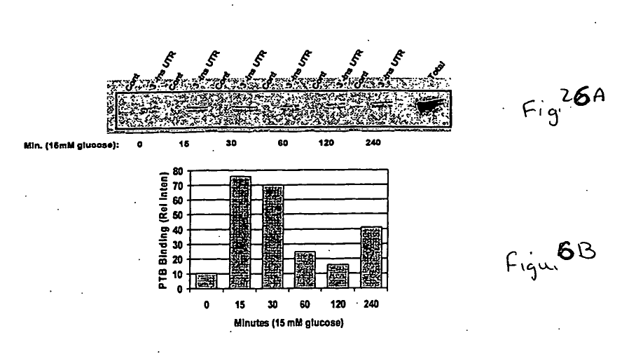

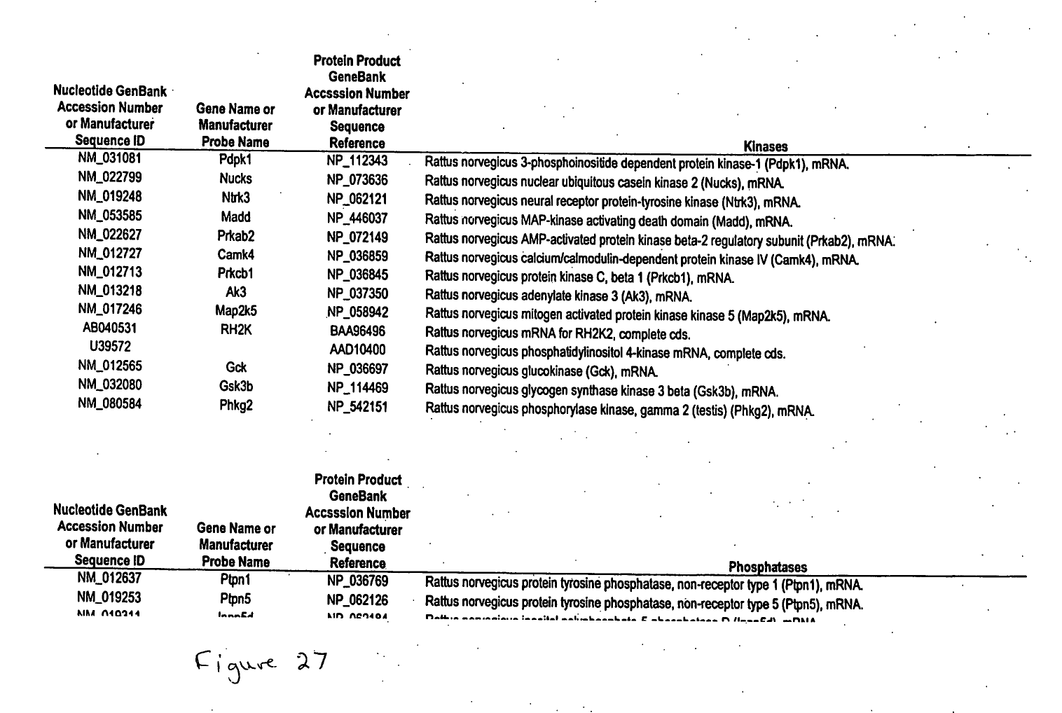

RIBOTRAP™ Binding Study: A biotinylated RNA oligonucleotide probe specific for the 3′-UTR of preproinsulin, 5′-gcccaccacuacccugaccaccccucugcaaugaauaaaaccuuugaaagagc-3′, and a biotinylated control RNA oligonucleotide probe, 5′-ugaauacaagcucacgacccacuacacaagcuaccagauacaacaacaagcauccacc-3′ were prebound to streptavidin agarose beads according to standard methods. For PTB binding, the salt concentration of INS-1 cell extracts was adjusted to 300 mM NaCl and 10-100 μl cell extract was incubated with the biotinylated oligonucleotide probes (1-50 μg) for 30 minutes to 12 hours. The beads were washed in RLB binding buffer (RLB/300 mM NaCl) and bound protein eluted in SDS-PAGE sample buffer according to standard methods. Detection of bound PTB by immunoblotting was carried out using a monoclonal antibody against PTB (Zymed, South San Francisco, Calif.). FIG. 26 shows the results of the immunoblot probed with the α-PTB monoclonal antibody, and indicates that glucose stimulates an acute but transient increase in PTB binding to the preproinsulin 3′-UTR. No binding was detected using the control RNA oligonucleotide.

### Example 9

### Identification of PTB Ribonomic Cluster using RAS™

The PTB ribonomic cluster was isolated and characterized using RAS™. Cell extracts were prepared from INS-1 cells that had been stepped-down in low glucose and then stimulated with high glucose media for 2 hours as described above in Examples 7 and 8. Cell extracts were prepared by harvesting cells in RLB buffer as described in Example 7. Following centrifugation, the salt concentration of the cell extracts was adjusted to 300 mM NaCl and 15 mM EDTA (RLB/NaCl/EDTA). These extracts (500 μg protein) were incubated with 10 μg of the anti-PTB monoclonal antibody α-PTB (Zymed, Cat# 32-4800, South San Francisco, Calif.) or 10 μg of a control IgG (Pierce Biotechnology, Rockford, Ill.) for 2 hours, followed by a 1 hour incubation with 30 μl of protein A sepharose (Pierce Biotechnology, Rockford, Ill.). The immunoprecipitates were washed 6 times in RLB/NaCl/EDTA. RNA was extracted and purified from the immunoprecipitates using PicoPure RNA isolation kits (Arcturus, Mountain View, Calif.). The purified RNA was quantified by RiboGreen analysis (Molecular Probes, Eugene, Oreg.) and the integrity of the samples was determined using a BioAnalyzer (Agilent, Palo Alto, Calif.). From these analyses, approximately 25-30 ng of nucleic acid was associated with the control IgG immunoprecipitates. In contrast, approximately 200-900 ng of nucleic acid was immunoprecipitated by the PTB antibody. In order to obtain enough RNA for microarray studies, samples of approximately 500 ng were subjected to two rounds of amplification using the MessageAmp kits and protocols (Ambion, Austin, Tex.) as described by the manufacturer. Microarray analysis was performed as described in Example 1.

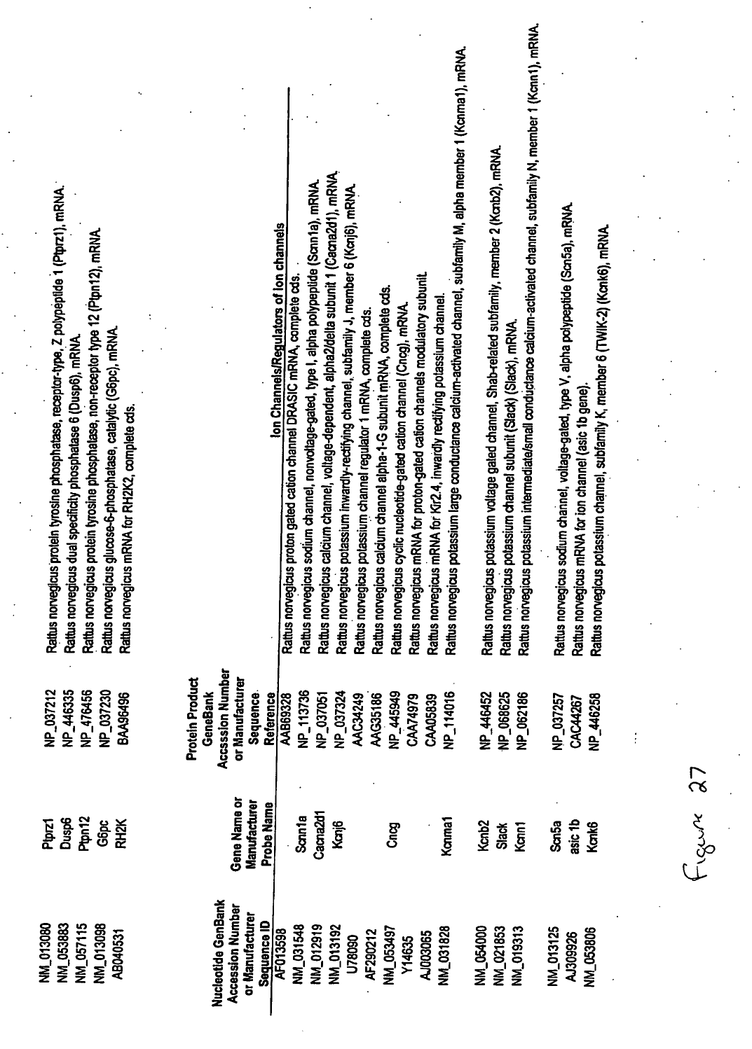

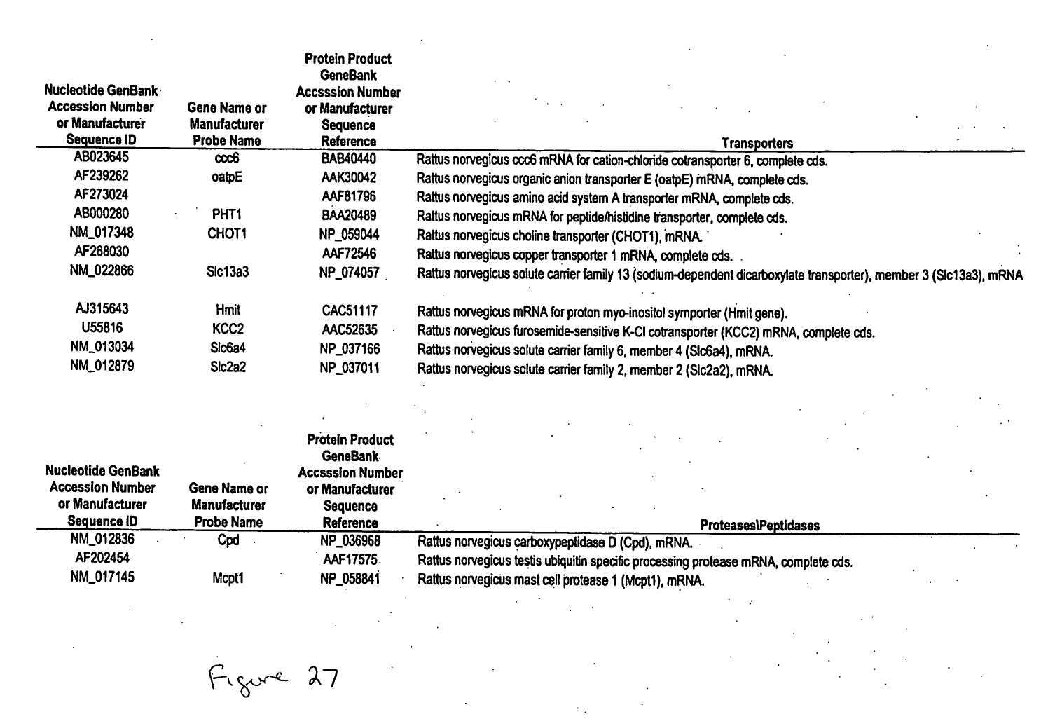

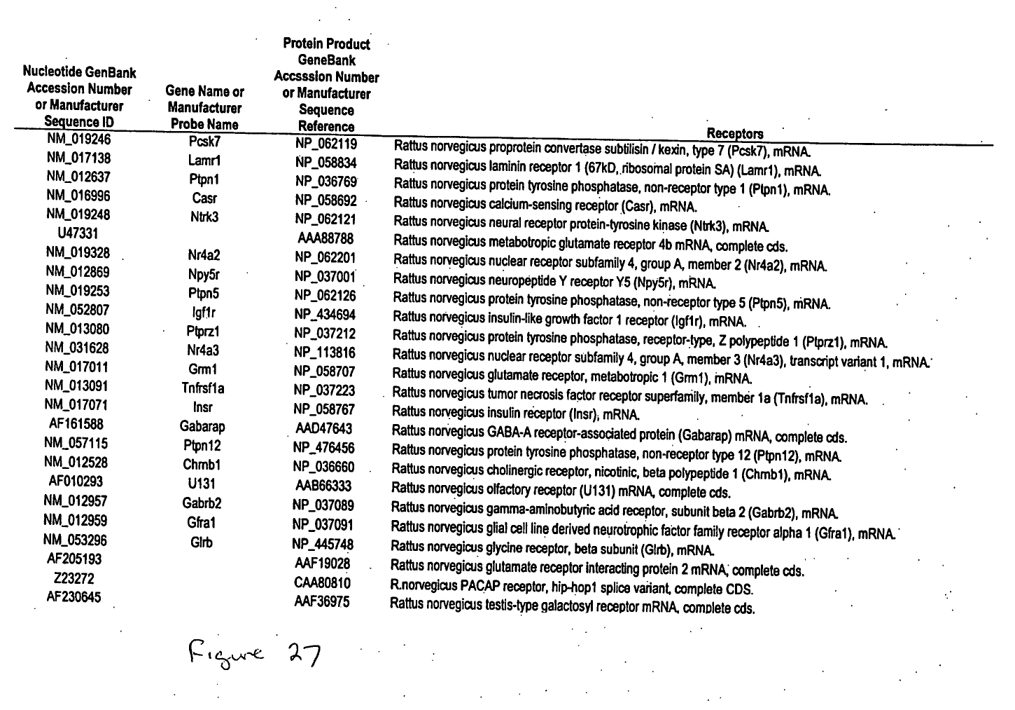

For purposes of examining potential therapeutic targets from the PTB-cluster, genes with ≧5× enrichment compared to amplified total RNAs were sorted into the drug target classes and are listed in FIG. 27.

### Example 10

### Use of RNAi-Mediated Gene Silencing of RNA Binding Proteins to Characterize RBP Clusters

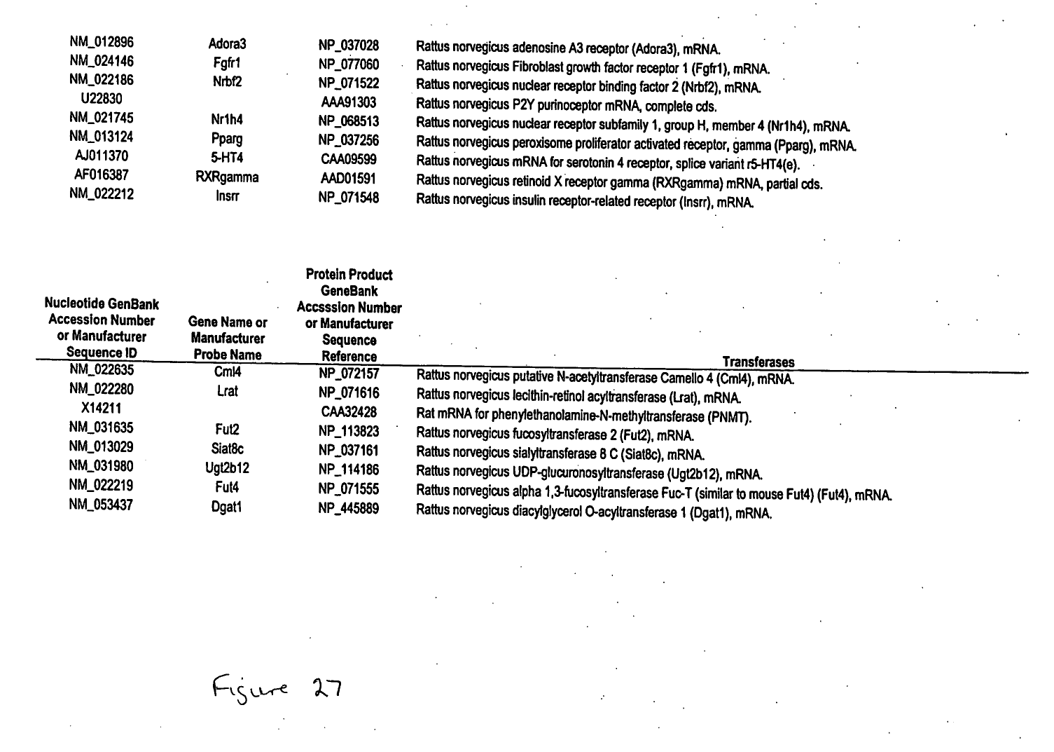

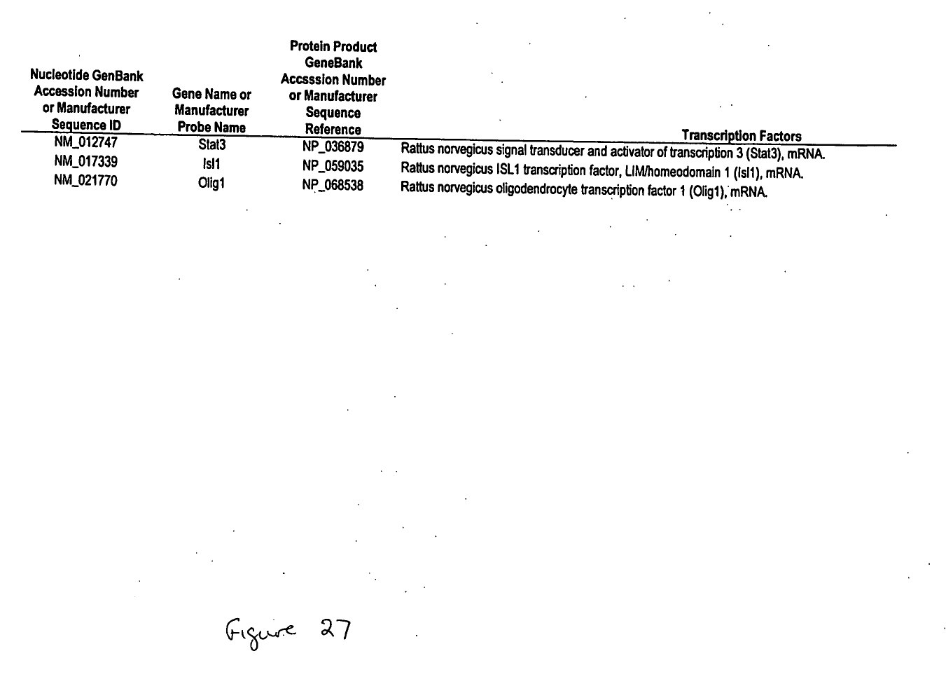

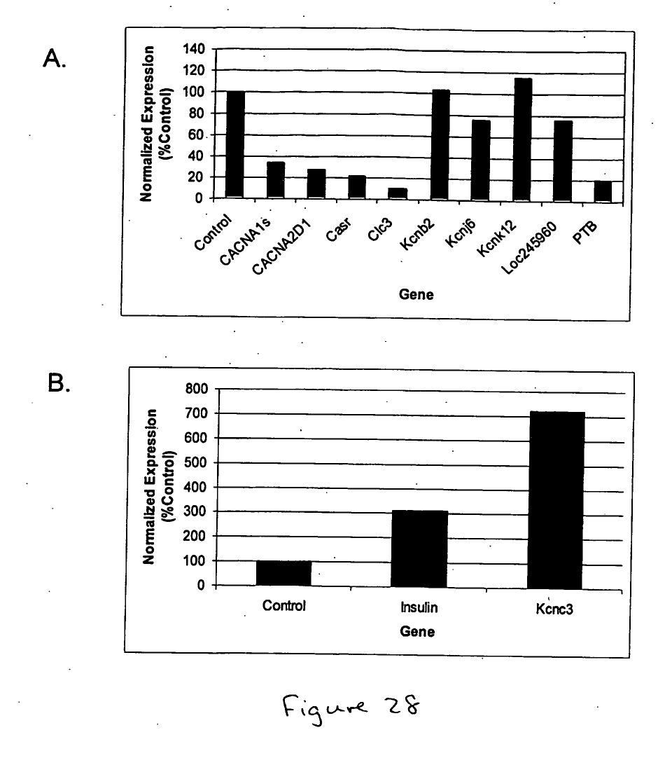

RNAi was used to inhibit PTB expression and to examine the effect of RNAi-mediated down-regulation of PTB expression on the expression of several genes within the PTB-cluster. INS-1 cells were plated in 24-well culture dishes, and incubated with fresh RPMI media containing 10% fetal bovine serum. TransitTKO transfection reagent (Dharmacon, Lafayette, Colo.), 2 μl, was incubated for 15 minute at room temperature with SmartPool™ siRNAs (Dharmacon, Lafayette, Colo., Cat# M-003841-00-05) targeted specifically to PTB at a concentration range to yield a final concentration of 1-50 nM siRNA on the cells. After a 24 hour incubation at 37° C., total RNA was isolated and used in QR-TPCR analysis. FIG. 28 illustrates the effect of PTB inhibition on the expression of PTB, preproinsulin, and nine additional genes found within the PTB-cluster. As indicated in FIG. 28A, there was an 80% reduction in PTB mRNA expression, confirming the action of the PTB specific RNAi. In addition, CACNA1S, CACNA2D1, Casr, C1c3, Kcnj6, AND Loc245960 and were significantly down-regulated as a result of PTB knockdown. FIG. 28B illustrates genes whose expression was up-regulated as a result of PTB knockdown. This includes insulin, which is up-regulated 3-fold.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### INCORPORATION BY REFERENCE

All publications and patent documents cited in this application are incorporated by reference in their entirety for all purposes to the same extent as if the contents of each individual publication or patent document was incorporated herein.

We claim:

1. A method of identifying a therapeutic target, the method comprising the steps of:
(a) measuring protein or RNA levels of at least one component of an isolated mRNA ribonucleoprotein (mRNP) complex in a first sample enriched for a cell comprising a first phenotype; and
(b) comparing the levels determined in step (a) to the levels of the protein or RNA levels of the component in a second sample enriched for a cell comprising a second phenotype,
wherein if the levels of the component in the first sample are different from the levels of the component in the second sample, the component, a nucleic acid that encodes the component, or a protein encoded by the component is a potential therapeutic target for the treatment of a disease.

2. The method of claim 1, wherein the cell comprising the first phenotype is selected from the group consisting of a mature adipocyte, a preadipocyte, pancreatic beta cell, a hepatocyte, a skeletal muscle cell, and a cardiac muscle cell.

3. The method of claim 1, wherein the cell comprising the first phenotype is a mature adipocyte and the cell comprising the second phenotype is a preadipocyte.

4. The method of claim 1, wherein the first phenotype is a disease related to glucose or lipid metabolism and the second phenotype is a normal phenotype.

5. The method of claim 1, wherein the first phenotype is selected from the group consisting of obesity, diabetes, hypoglycemia, glucotoxicity, lipidtoxicity, insulin-resistance, hyperlipidemia, and lipodystrophy.

6. The method of claim 1, wherein the component is selected from the group consisting of an RNA binding protein, an RNA, and an mRNP-associated protein.

7. The method of claim 1, the method further comprising the step of:
(c) treating the sample in step (a) with an agent prior to measuring the protein or RNA levels of the component, wherein the agent alters the levels of at least one component of a glucose metabolic or a lipid metabolic pathway.

8. The method of claim 7, wherein the agent is selected from the group consisting of insulin, glucose, insulin-like growth factor-1 (IGF-1), a β-adrenergic agonist, glucose, glucagon-like peptide-1 (GLP-1), fatty acid, a peroxisome proliferator activated receptor (PPAR) ligand, and insulin-like growth factor 2 (IGF-2).

9. The method of claim 7, wherein the agent is a test therapeutic.

10. The method of claim 7, wherein the agent is selected from the group consisting of a nucleic acid, a protein, a peptide, or a small molecule.

11. The method of claim 1 or 7, further comprising the step of isolating the component, a nucleic acid encoding the component, or a protein encoded by the component.

12. The method of claim 1, wherein the component is Polypyrimidine Tract Binding Protein.

13. The method of claim 1, wherein the RNA binding protein is selected from the group consisting of the RNA binding proteins identified in FIG. 10 to FIG. 22.

14. The method of claim 1, wherein the component comprises a tag.

15. The method of claim 1, wherein the component is an mRNA that encodes a protein selected from the group consisting of a kinase, a transporter, a phosphatase, channel protein, a protease, a receptor, a transcription factor, and a transferase.

16. The method of claim 1, wherein the component is selected from the group consisting of 3-phosphoinositide dependent protein kinase-1, nuclear ubiquitous casein kinase 2, neural receptor protein-tyrosine kinase, MAP-kinase activating death domain, AMP-activated protein kinase beta-2 regulatory subunit, calcium/calmodulin-dependent protein kinase IV, Protein kinase C beta, adenylate kinase 3, mitogen activated protein kinase kinase 5,6-phosphofructo-2-kinase/fructose-2,6-bisphosphatase 2, phosphatidylinositol 4-kinase, Glucokinase, glycogen synthase kinase 3 beta, phosphorylase kinase (gamma 2, testis), protein tyrosine phosphatase (non-receptor type 1), protein tyrosine phosphatase (non-receptor type 5), inositol polyphosphate-5-phosphatase D, Protein tyrosine phosphatase (receptor-type, zeta polypeptide), dual specificity phosphatase 6, protein tyrosine phosphatase (non-receptor type 12), glucose-6-phosphatase (catalytic), 6-phosphofructo-2-kinase/fructose-2,6-bisphosphatase 2, proton gated cation channel DRASIC, Sodium channel (nonvoltage-gated 1, alpha (epithelial)), calcium channel (voltage-dependent, alpha2/delta subunit 1), Potassium inwardly-rectifying (channel, subfamily J, member 6), potassium channel regulator 1, calcium channel (voltage-dependent, T type, alpha 1G subunit), cyclic nucleotide-gated cation channel, amiloride-sensitive cation channel 1, potassium inwardly-rectifying channel J14, potassium large conductance calcium-activated channel (subfamily M, alpha member 1), potassium voltage gated channel (Shab-related subfamily, member 2), potassium channel subunit (Slack), potassium intermediate/small conductance calcium-activated channel (subfamily N, member 1), Sodium channel (voltage-gated, type V, alpha polypeptide), amiloride-sensitive cation channel 2 (neuronal), potassium channel (subfamily K, member 6 (TWIK-2)), cation-chloride cotransporter 6, solute carrier family 21 (organic anion transporter, member 12), amino acid transporter system A2, peptide/histidine transporter, choline transporter, solute carrier family 31 (copper transporters, member 1), solute carrier family 13 (sodium-dependent dicarboxylate transporter), solute carrier family 2 (facilitated glucose transporter, member 13), solute carrier family 12 (potassium-chloride transporter, member 5), Solute carrier family 6 (neurotransmitter transporter, serotonin, member 4), Solute carrier family 2 A2 (glucose transporter, type 2), carboxypeptidase D, ubiquitin specific protease 2, mast cell protease 1, proprotein convertase subtilisin/kexin, type 7, laminin receptor 1 (67 kD, ribosomal protein SA), protein tyrosine phosphatase (non-receptor type 1), calcium-sensing receptor, neural receptor protein-tyrosine kinase, glutamate receptor (metabotropic 4), nuclear receptor subfamily 4 (group A, member 2), Neuropeptide Y5 receptor, protein tyrosine phosphatase (non-receptor type 5), insulin-like growth factor 1 receptor, Protein tyrosine phosphatase (receptor-type, zeta polypeptide), nuclear receptor subfamily 4 (group A, member 3), glutamate receptor (metabotropic 1), Tumor necrosis factor receptor superfamily (member 1a), insulin receptor, gamma-aminobutyric acid receptor associated protein, protein tyrosine phosphatase, non-receptor type 12, cholinergic receptor (nicotinic, beta polypeptide 1), olfactory receptor (U 131), Gamma-aminobutyric acid receptor beta 2, glial cell line derived neurotrophic factor family receptor alpha 1, Glycine receptor beta, glutamate receptor interacting protein 2, adenylate cyclase activating polypeptide 1 receptor 1, asialoglycoprotein receptor 2, adenosine A3 receptor, Fibroblast growth factor receptor 1, nuclear receptor binding factor 2, purinergic receptor P2Y (G-protein coupled 1), nuclear receptor subfamily 1 (group H, member 4), peroxisome proliferator activator receptor (gamma), 5 hydroxytryptamine (serotonin) receptor 4, retinoid X receptor gamma, insulin receptor-related receptor, putative N-acetyltransferase Camello 4, lecithin-retinol acyltransferase, Phenylethanolamine N-methyltransferase, fucosyltransferase 2, Sialyltransferase 8 (GT3 alpha 2,8-sialyltransferase) C, UDP-glucuronosyltransferase, alpha 1,3-fucosyltransferase Fuc-T (similar to mouse Fut4), diacylglycerol O-acyltransferase 1, signal transducer and activator of transcription 3, ISL1 transcription factor (LIM/homeodomain), and oligodendrocyte transcription factor 1.

17. The method of claim 16, wherein the protein is encoded by a gene selected from the group consisting of CNCG, CACNA2D1, KCNC3, and KCNB2.

18. A method for identifying a therapeutic target for the treatment of aberrant glucose metabolism or lipid metabolism, the method comprising the steps of:
(a) measuring RNA or protein levels of at least one component of an isolated mRNP complex in a first cell sample; and
(b) comparing RNA or protein levels determined in step (a) to the RNA or protein levels of the component from a second cell sample,
wherein if the levels of the component in the first sample are different from the levels of the component in the second sample, the component, a nucleic acid that encodes the component, or a protein encoded by the component is a potential therapeutic target for the treatment of the disease.

19. The method of claim 18, wherein the first cell sample is from an individual at risk of having a disease or who has a disease and the second cell sample is from a normal or healthy individual.

20. A method for identifying a therapeutic target related to the treatment of a disease, the method comprising the steps of:
(a) measuring RNA or protein levels of at least one component of an isolated mRNP complex in a sample that has been treated with an agent that alters the expression of a component of a glucose metabolic or lipid metabolic pathway; and
(b) comparing RNA or protein levels determined in step (a) to the RNA or protein levels of the component in an untreated control sample,
wherein if the levels of the component in the first sample are different from the levels of the component in the second sample, the component, a nucleic acid that encodes the component, or a protein encoded by the component is a potential therapeutic target for the treatment of the disease.

21. A method for identifying a gene or gene product involved in a physiological pathway in a cell, the method comprising the steps of:
a. isolating an mRNP complex comprising at least one component that participates in a physiological pathway;
b. identifying at least one additional component of the isolated mRNP complex,
wherein the additional component is also involved in a physiological pathway.

22. The method of claim 21, wherein the physiological pathway comprises a metabolic pathway or a regulatory pathway.

23. The method of claim 21, further comprising the step of confirming the activity of the additional component by inhibiting the expression of the additional component in a cell and determining the effect of the inhibition on metabolism.

24. The method of claim 23, wherein the inhibition step comprises inhibiting gene expression of the additional component using an agent selected from the group consisting of an RNAi, an antisense RNA, a ribozyme, and a PNA.

25. A method for identifying an agent that alters a physiological pathway, the method comprising the steps of:
a. subjecting a cell sample to an agent;
b. isolating an mRNP complex comprising at least one component that participates in a physiological pathway from the sample;
c. measuring the RNA or protein levels of at least one component of the isolated mRNP complex,
d. comparing the RNA or protein levels of step (c) to the RNA or protein levels of the component isolated from an untreated control sample,
wherein differential expression of the component in the agent-treated sample compared to the untreated control sample is indicative that the agent regulates the physiological pathway.

26. The method of claim 25, wherein the agent interacts with or regulates a component of the physiological pathway.

27. The method of claim 25, wherein the agent inhibits a physiological pathway.

28. The method of claim 25, wherein the agent enhances a physiological pathway.

29. The method of claim 25, wherein the physiological pathway is an insulin production pathway or a lipogenesis pathway.

30. A method for identifying a protein that regulates glucose metabolism, the method comprising the steps of:
a. measuring the expression in an isolated mRNP complex of at least one gene product of a cell involved in glucose metabolism, wherein the gene product is selected from the group consisting of an RNA binding protein, an mRNA associated with said RNA binding protein, or an mRNP complex-associated protein;
b. treating the cell with an agent selected from the group consisting of insulin, glucose, insulin-like growth factor-1 (IGF-1), a β-adrenergic agonist, glucose, glucagon-like peptide-1 (GLP-1), fatty acid, a peroxisome proliferator activated receptor (PPAR) ligand, and insulin-like growth factor 2 (IGF-2); and
c. measuring the expression of the gene product after treatment, wherein a difference in expression of the gene product after treatment compared to expression of the gene product before treatment is indicative that the protein regulates glucose metabolism.

31. A method for identifying an agent that regulates insulin production, the method comprising the steps of:
a. contacting a cell involves in insulin production with a nucleic acid capable of binding to at least one protein, wherein the protein is capable of binding to a 3′ untranslated region or a 5′ untranslated region of a preproinsulin mRNA;
b. separating the nucleic acid from the protein; and
c. identifying the protein.

32. The method of claim 31, wherein the protein binds to a nucleic acid comprising a sequence selected from the group consisting of 5′-gaauaaaaccuuugaaagagcacuac-3′,5′-cccaccacuacccuguccaccccucugcaaug-3′, and 5′-agccctaagtgaccagctacagtcggaaaccatcagcaagcaggtcattgttccaac-3′.

33. An mRNP complex-associated with at least one of glucose or lipid metabolism, wherein the mRNP complex comprises a polypyrimidine tract binding (PTB) protein, and at least one mRNA associated with the polypyrimidine tract binding protein.

34. A method for identifying a component of an mRNP complex, the method comprising the steps of:
(a) transfecting a cell sample with a nucleic acid that inhibits the expression of an RNA binding protein;
(b) isolating total RNA from the cell sample and from a control sample;
(c) identifying RNAs that have altered expression in the nucleic acid-transfected sample compared to the control sample.

35. The method of any one of claims 1, 7, 18, and 20, wherein the disease is related to aberrant glucose or lipid metabolism.

36. The method of claim 21 or 25, wherein the physiological pathway comprises a glucose or lipid metabolic pathway.

37. The method of any one of claims 1, 17, 20, 25, and 30, wherein at least one of said measuring and said comparing steps comprises the use of an array.


SEQUENCE LISTING (10 sequences)

SEQ ID NO: 1 - RNA, 26 nt, Artificial Sequence
  Description of Artificial Sequence Synthetic oligonucleotide
gaauaaaaccuuugaaagagcacuac

SEQ ID NO: 2 - RNA, 32 nt, Artificial Sequence
  Description of Artificial Sequence Synthetic oligonucleotide
cccaccacuacccuguccaccccucugcaaug

SEQ ID NO: 3 - DNA, 57 bp, Artificial Sequence
  Description of Artificial Sequence Synthetic oligonucleotide
agccctaagtgaccagctacagtcggaaaccatcagcaagcaggtcattgttccaac

SEQ ID NO: 4 - protein, 13 aa, Artificial Sequence
  Description of Artificial Sequence Synthetic peptide
MASMTGGQQMGRC

SEQ ID NO: 5 - RNA, 53 nt, Artificial Sequence
  Description of Artificial Sequence Synthetic oligonucleotide′
gcccaccacuacccugaccaccccucugcaaugaauaaaaccuuugaaagagc

SEQ ID NO: 6 - RNA, 58 nt, Artificial Sequence
  Description of Artificial Sequence Synthetic oligonucleotide′
ugaauacaagcucacgacccacuacacaagcuaccagauacaacaacaagcauccacc

SEQ ID NO: 7 - protein, 6 aa, Artificial Sequence
  Description of Artificial Sequence Synthetic 6xHis tag
HHHHHH

SEQ ID NO: 8 - protein, 4 aa, Homo sapiens
DEAD

SEQ ID NO: 9 - protein, 4 aa, Homo sapiens
DEAH

SEQ ID NO: 10 - protein, 4 aa, Homo sapiens
  MOD_RES (4): Asp or His
DEAX